(19) United States
(12) Patent Application Publication
(10) Pub. No.: US 2023/0021562 A1
(43) Pub. Date: Jan. 26, 2023

(54) MACROCYLIC INDOLE DERIVATIVES MCL-1 INHIBITORS

(71) Applicant: Janssen Pharmaceutica NV, Beerse (BE)

(72) Inventors: Frederik Jan Rita ROMBOUTS, Wilrijk (BE); Benoit Christian Albert Ghi De BOECK, Genval (BE)

(21) Appl. No.: 17/778,523

(22) PCT Filed: Nov. 20, 2020
(86) PCT No.: PCT/EP2020/082900

§ 371 Date: May 20, 2022

(30) Foreign Application Priority Data
Nov. 21, 2019 (EP) 19210710.0

(51) Int. Cl. C07D 515/22

(52) U.S. Cl. CPC C07D 515/22

(57) ABSTRACT

The present invention relates to pharmaceutical agents useful for therapy and/or prophylaxis in a subject, pharmaceutical composition comprising such compounds, and their use as MCL-1 inhibitors, useful for treating diseases such as cancer.

### FIELD OF THE INVENTION

The present invention relates to pharmaceutical agents useful for therapy and/or prophylaxis in a subject, pharmaceutical composition comprising such compounds, and their use as MCL-1 inhibitors, useful for treating or preventing diseases such as cancer.

### BACKGROUND OF THE INVENTION

Cellular apoptosis or programmed cell death is critical to the development and homeostasis of many organs including the hematopoietic system. Apoptosis can be initiated via the extrinsic pathway, which is mediated by death receptors, or by the intrinsic pathway using the B cell lymphoma (BCL-2) family of proteins. Myeloid cell leukemia-1 (MCL-1) is a member of the BCL-2 family of cell survival regulators and is a critical mediator of the intrinsic apoptosis pathway. MCL-1 is one of five principal anti-apoptotic BCL-2 proteins (MCL-1, BCL-2, BCL-XL, BCL-w, and BFL1/A1) responsible for maintaining cell survival. MCL-1 continuously and directly represses the activity of the pro-apoptotic BCL-2 family proteins Bak and Bax and indirectly blocks apoptosis by sequestering BH3 only apoptotic sensitizer proteins such as Bim and Noxa. The activation of Bak/Bax following various types of cellular stress leads to aggregation on the mitochondrial outer membrane and this aggregation facilitates pore formation, loss of mitochondrial outer membrane potential, and subsequent release of cytochrome C into the cytosol. Cytosolic cytochrome C binds Apaf-1 and initiates recruitment of procaspase 9 to form apoptosome structures (Cheng et al. eLife 2016; 5: e17755). The assembly of apoptosomes activates the executioner cysteine proteases 3/7 and these effector caspases then cleave a variety of cytoplasmic and nuclear proteins to induce cell death (Julian et al. Cell Death and Differentiation 2017; 24, 1380-1389).

Avoiding apoptosis is an established hallmark of cancer development and facilitates the survival of tumor cells that would otherwise be eliminated due to oncogenic stresses, growth factor deprivation, or DNA damage (Hanahan and Weinberg. Cell 2011; 1-44). Thus, unsurprisingly, MCL-1 is highly upregulated in many solid and hematologic cancers relative to normal non-transformed tissue counterparts. The overexpression of MCL-1 has been implicated in the pathogenesis of several cancers where it correlated with poor outcome, relapse, and aggressive disease. Additionally, overexpression of MCL-1 has been implicated in the pathogenesis of the following cancers: prostate, lung, pancreatic, breast, ovarian, cervical, melanoma, B-cell chronic lymphocytic leukemia (CLL), acute myeloid leukemia (AML), and acute lymphoblastic leukemia (ALL). The human MCL-1 genetic locus (1q21) is frequently amplified in tumors and quantitatively increases total MCL-1 protein levels (Beroukhim et al. Nature 2010; 463 (7283) 899-905). MCL-1 also mediates resistance to conventional cancer therapeutics and is transcriptionally upregulated in response to inhibition of BCL-2 function (Yecies et al. Blood 2010; 115 (16)3304-3313).

A small molecule BH3 inhibitor of BCL-2 has demonstrated clinical efficacy in patients with chronic lymphocytic leukemia and is FDA approved for patients with CLL or AML (Roberts et al. NEJM 2016; 374:311-322). The clinical success of BCL-2 antagonism led to the development of several MCL-1 BH3 mimetics that show efficacy in preclinical models of both hematologic malignancies and solid tumors (Kotschy et al. Nature 2016; 538 477-486, Merino et al. Sci. Transl. Med; 2017 (9)).

MCL-1 regulates several cellular processes in addition to its canonical role in mediating cell survival including mitochondrial integrity and non-homologous end joining following DNA damage (Chen et al. JCI 2018; 128(1):500-516). The genetic loss of MCL-1 shows a range of phenotypes depending on the developmental timing and tissue deletion. MCL-1 knockout models reveal there are multiple roles for MCL-1 and loss of function impacts a wide range of phenotypes. Global MCL-1-deficient mice display embryonic lethality and studies using conditional genetic deletion have reported mitochondrial dysfunction, impaired activation of autophagy, reductions in B and T lymphocytes, increased B and T cell apoptosis, and the development of heart failure/cardiomyopathy (Wang et al. Genes and Dev 2013; 27 1351-1364, Steimer et al. Blood 2009(113) 2805-2815).

WO2018178226 discloses MCL-1 inhibitors and methods of use thereof.

WO2017182625 discloses macrocyclic MCL-1 inhibitors for treating cancer.

WO2018178227 discloses the synthesis of MCL-1 inhibitors.

WO2007008627 discloses substituted phenyl derivatives as inhibitors of the activity of anti-apoptotic MCL-1 protein.

WO2008130970 discloses 7-nonsubstituted indole MCL-1 inhibitors.

WO2008131000 discloses 7-substituted indole MCL-1 inhibitors.

WO2020063792 discloses indole macrocyclic derivatives.

CN110845520 discloses macrocyclic indoles as MCL-1 inhibitors.

WO2020103864 discloses macrocyclic indoles as MCL-1 inhibitors.

WO2020151738 discloses macrocyclic fused pyrrazoles as MCL-1 inhibitors.

WO2020185606 discloses macrocyclic compounds as MCL-1 inhibitors.

There remains a need for MCL-1 inhibitors, useful for the treatment or prevention of cancers such as prostate, lung, pancreatic, breast, ovarian, cervical, melanoma, B-cell chronic lymphocytic leukemia (CLL), acute myeloid leukemia (AML), and acute lymphoblastic leukemia (ALL).

### SUMMARY OF THE INVENTION

The present invention concerns novel compounds of Formula (I):

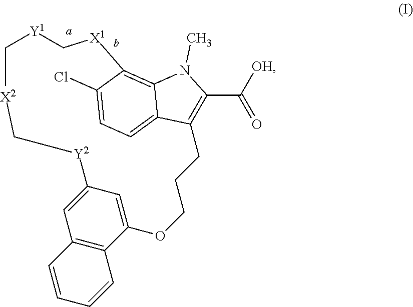

and the tautomers and the stereoisomeric forms thereof, wherein

X¹ represents

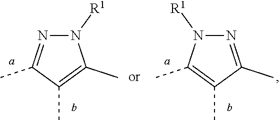

wherein ‘a’ and ‘b’ indicate how variable X¹ is attached to the remainder of the molecule;

X² represents

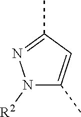

which can be attached to the remainder of the molecule in both directions;

R¹ and R² each independently represent hydrogen; methyl; or C₂₋₆alkyl optionally substituted with one substituent selected from the group consisting of Het¹, —OR³, and —NR^{4a}R^{4b}; provided that at least one of R¹ and R² is other than methyl;

R³ represents hydrogen, C₁₋₄alkyl, or —C₂₋₄alkyl-O—C₁₋₄alkyl;

R^{4a} and R^{4b} are each independently selected from the group consisting of hydrogen and C₁₋₄alkyl;

Het¹ represents morpholinyl or tetrahydropyranyl;

Y¹ represents —S—, —S(═O)₂— or —N(R^{x})—;

R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;

Y² represents —S— or —S(═O)₂—;

and the pharmaceutically acceptable salts and the solvates thereof.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer.

In a particular embodiment, the invention relates to a compound of Formula (I), a pharmaceutically acceptable salt, or a solvate thereof, for use in the treatment or in the prevention of cancer.

The invention also relates to the use of a compound of Formula (I), a pharmaceutically acceptable salt, or a solvate thereof, in combination with an additional pharmaceutical agent for use in the treatment or prevention of cancer.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer.

Additionally, the invention relates to a method of treating or preventing a cell proliferative disease in a subject which comprises administering to the said subject an effective amount of a compound of Formula (I), a pharmaceutically acceptable salt, or a solvate thereof, as defined herein, or a pharmaceutical composition or combination as defined herein.

### DETAILED DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain fully saturated hydrocarbon radical having from 1 to 4 carbon atoms, such as methyl, ethyl, n-propyl, isopropyl, n-butyl, s-butyl, t-butyl and the like.

The term ‘C₁₋₆alkyl’ as used herein as a group or part of a group represents a straight or branched chain fully saturated hydrocarbon radical having from 1 to 6 carbon atoms, such as methyl, ethyl, n-propyl, isopropyl, n-butyl, s-butyl, t-butyl, n-pentyl, n-hexyl and the like.

The term ‘C₂₋₆alkyl’ as used herein as a group or part of a group represents a straight or branched chain fully saturated hydrocarbon radical having from 2 to 6 carbon atoms, such as ethyl, n-propyl, isopropyl, n-butyl, s-butyl, t-butyl, n-pentyl, n-hexyl and the like.

The term ‘C₃₋₆cycloalkyl’ as used herein as a group or part of a group defines a fully saturated, cyclic hydrocarbon radical having from 3 to 6 carbon atoms, such as cyclopropyl, cyclobutyl, cyclopentyl and cyclohexyl.

It will be clear for the skilled person that S(═O)₂ or SO₂ represents a sulfonyl moiety.

It will be clear for the skilled person that CO or C(═O) represents a carbonyl moiety.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture.

Combinations of substituents and/or variables are permissible only if such combinations result in chemically stable compounds. ‘Stable compound’ is meant to indicate a compound that is sufficiently robust to survive isolation to a useful degree of purity from a reaction mixture.

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

It will be clear for the skilled person that, unless otherwise is indicated or is clear from the context, a substituent on a heterocyclyl group may replace any hydrogen atom on a ring carbon atom or on a ring heteroatom (e.g. a hydrogen on a nitrogen atom may be replaced by a substituent).

Unless otherwise specified or clear from the context, aromatic rings and heterocyclyl groups, can be attached to the remainder of the molecule of Formula (I) through any available ring carbon atom (C-linked) or nitrogen atom (N-linked).

It will be clear for the skilled person that

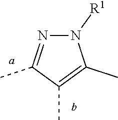

is an alternative representation for a

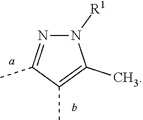

It will be clear for the skilled person that

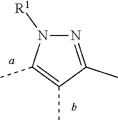

is an alternative presentation for

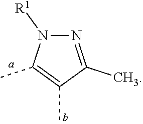

It will be clear that a Compound of Formula (I) includes Compounds of Formula (I-x) and (I-y) (both directions of X² being

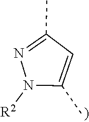

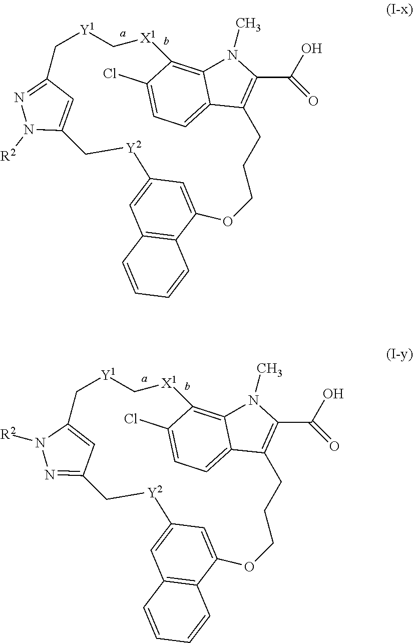

When any variable occurs more than one time in any constituent, each definition is independent.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, or subject (e.g., human) that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

In particular, the compounds disclosed herein possess axial chirality, by virtue of restricted rotation around a biaryl bond and as such may exist as mixtures of atropisomers. When a compound is a pure atropisomer, the stereochemistry at each chiral center may be specified by either Rₐ or Sₐ. Such designations may also be used for mixtures that are enriched in one atropisomer. Further description of atropisomerism and axial chirality and rules for assignment of configuration can be found in Eliel, E. L. & Wilen, S. H. ‘Stereochemistry of Organic Compounds’ John Wiley and Sons, Inc. 1994.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light. Optically active (Rₐ)- and (Sₐ)-atropisomers may be prepared using chiral synthons, chiral reagents or chiral catalysts, or resolved using conventional techniques well known in the art, such as chiral HPLC.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer; when a compound of Formula (I) is for instance specified as Rₐ, this means that the compound is substantially free of the Sₐ atropisomer.

Pharmaceutically acceptable salts, in particular pharmaceutically acceptable additions salts, include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I), and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, N-oxides and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S ¹⁸F, ³⁶Cl, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C and ¹⁸F. More preferably, the isotope is ²H. In particular, deuterated compounds are intended to be included within the scope of the present invention.

Certain isotopically-labeled compounds of the present invention (e.g., those labeled with 3H and ¹⁴C) may be useful for example in substrate tissue distribution assays. Tritiated (3H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

The present invention relates in particular to compounds of Formula (I) as defined herein, and the tautomers and the stereoisomeric forms thereof, wherein

X¹ represents

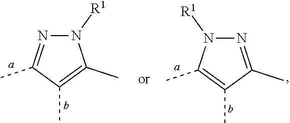

wherein ‘a’ and ‘b’ indicate how variable X¹ is attached to the remainder of the molecule;

X² represents

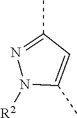

which can be attached to the remainder of the molecule in both directions;

R¹ and R² each independently represent hydrogen, methyl, or C₂₋₆alkyl optionally substituted with one substituent selected from the group consisting of Het¹, —OR³, and —NR^{4a}R^{4b}; provided that at least one of R¹ and R² is other than methyl;

R³ represents hydrogen, C₁₋₄alkyl, or —C₂₋₄alkyl-O—C₁₋₄alkyl; R^{4a} and R^{4b} are C₁₋₄alkyl;

Het¹ represents morpholinyl or tetrahydropyranyl; in particular 1-morpholinyl or 4-tetrahydropyranyl;

Y¹ represents —S—, —S(═O)₂— or —N(R^{x})—;

R^{x} represents methyl;

Y² represents —S— or —S(═O)₂—:

and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein

Y¹ represent —N(R^{x})—.

In an embodiment, the present invention relates to those compounds of Formula (I) and the pharmaceutically acceptable salts, and the solvates thereof, or any subgroup thereof as mentioned in any of the other embodiments, wherein

Y¹ represent —N(R^{x})—; and R^{x} represents hydrogen.

In an embodiment, the present invention relates to those compounds of Formula (I) and the pharmaceutically acceptable salts, and the solvates thereof, or any subgroup thereof as mentioned in any of the other embodiments, wherein

Y¹ represent —N(R^{x})—; and R^{x} represents methyl.

In an embodiment, the present invention relates to those compounds of Formula (I) and the pharmaceutically acceptable salts, and the solvates thereof, or any subgroup thereof as mentioned in any of the other embodiments, wherein

X¹ represents

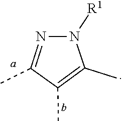

In an embodiment, the present invention relates to those compounds of Formula (I) and the pharmaceutically acceptable salts, and the solvates thereof, or any subgroup thereof as mentioned in any of the other embodiments, wherein

X¹ represents

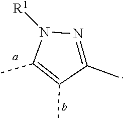

In an embodiment, the present invention relates to those compounds of Formula (I) and the pharmaceutically acceptable salts, and the solvates thereof, or any subgroup thereof as mentioned in any of the other embodiments, wherein R¹ represents hydrogen.

In an embodiment, the present invention relates to those compounds of Formula (I) and the pharmaceutically acceptable salts, and the solvates thereof, or any subgroup thereof as mentioned in any of the other embodiments, wherein R¹ represents methyl or C₂₋₆alkyl.

In an embodiment, the present invention relates to those compounds of Formula (I) and the pharmaceutically acceptable salts, and the solvates thereof, or any subgroup thereof as mentioned in any of the other embodiments, wherein R¹ represents C₂₋₆alkyl.

In an embodiment, the present invention relates to those compounds of Formula (I) and the pharmaceutically acceptable salts, and the solvates thereof, or any subgroup thereof as mentioned in any of the other embodiments, wherein R¹ represents methyl.

In an embodiment, the present invention relates to those compounds of Formula (I) and the pharmaceutically acceptable salts, and the solvates thereof, or any subgroup thereof as mentioned in any of the other embodiments, wherein R¹ represents C₂₋₆alkyl substituted with one substituent selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein R¹ represents C₂₋₆alkyl substituted with one substituent selected from the group consisting of Het¹, —OR³, and —NR^{4a}R^{4b}; and

R^{x} represents methyl.

In an embodiment, the present invention relates to those compounds of Formula (I) and the pharmaceutically acceptable salts, and the solvates thereof, or any subgroup thereof as mentioned in any of the other embodiments, wherein R² represents C₂₋₆alkyl substituted with one substituent selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein R² represents C₂₋₆alkyl substituted with one substituent selected from the group consisting of Het¹, —OR³, and —NR^{4a}R^{4b}; and

R^{x} represents methyl.

In an embodiment, the present invention relates to those compounds of Formula (I) and the pharmaceutically acceptable salts, and the solvates thereof, or any subgroup thereof as mentioned in any of the other embodiments, wherein R² represents hydrogen.

In an embodiment, the present invention relates to those compounds of Formula (I) and the pharmaceutically acceptable salts, and the solvates thereof, or any subgroup thereof as mentioned in any of the other embodiments, wherein R² represents methyl or C₂₋₆alkyl.

In an embodiment, the present invention relates to those compounds of Formula (I) and the pharmaceutically acceptable salts, and the solvates thereof, or any subgroup thereof as mentioned in any of the other embodiments, wherein R² represents C₂₋₆alkyl.

In an embodiment, the present invention relates to those compounds of Formula (I) and the pharmaceutically acceptable salts, and the solvates thereof, or any subgroup thereof as mentioned in any of the other embodiments, wherein R² represents methyl.

In an embodiment, the present invention relates to those compounds of Formula (I) and the pharmaceutically acceptable salts, and the solvates thereof, or any subgroup thereof as mentioned in any of the other embodiments, wherein R¹ and R² each independently represent methyl; or C₂₋₆alkyl optionally substituted with one substituent selected from the group consisting of Het¹, —OR³, and —NR^{4a}R^{4b}; provided that at least one of R¹ and R² is other than methyl.

In an embodiment, the present invention relates to those compounds of Formula (I) and the pharmaceutically acceptable salts, and the solvates thereof, or any subgroup thereof as mentioned in any of the other embodiments, wherein R¹ and R² each independently represent hydrogen; methyl; or C₂₋₆alkyl optionally substituted with one substituent selected from the group consisting of Het¹, —OR³, and —NR^{4a}R^{4b}; provided that at least one of R¹ and R² is C₂₋₆alkyl substituted with one substituent selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein R¹ and R² each independently represent hydrogen; methyl; or C₂₋₆alkyl optionally substituted with one substituent selected from the group consisting of Het¹, —OR³, and —NR^{4a}R^{4b}; provided that at least one of R¹ and R² is C₂₋₆alkyl substituted with one substituent selected from the group consisting of Het¹, —OR³, and —NR^{4a}R^{4b}; and

R^{x} represents methyl. In an embodiment, the present invention relates to those compounds of Formula (I) and the pharmaceutically acceptable salts, and the solvates thereof, or any subgroup thereof as mentioned in any of the other embodiments, wherein R¹ and R² each independently represent hydrogen; or C₂₋₆alkyl optionally substituted with one substituent selected from the group consisting of Het¹, —OR³, and —NR^{4a}R^{4b}In an embodiment, the present invention relates to those compounds of Formula (I) and the pharmaceutically acceptable salts, and the solvates thereof, or any subgroup thereof as mentioned in any of the other embodiments, wherein R¹ and R² each independently represent hydrogen; or C₂₋₆alkyl substituted with one substituent selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein R¹ represents methyl; or C₂₋₆alkyl; and

R² represents C₂₋₆alkyl substituted with one substituent selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one substituent selected from the group consisting of Het¹, —OR³, and —NR^{4a}R^{4b}; and

R² represents methyl, or C₂₋₆alkyl.

In an embodiment, the present invention relates to those compounds of Formula (I) and the pharmaceutically acceptable salts, and the solvates thereof, or any subgroup thereof as mentioned in any of the other embodiments, wherein

R¹ represents methyl; or C₂₋₆alkyl optionally substituted with one substituent selected from the group consisting of Het¹, —OR³, and —NR^{4a}R^{4b}; and

R² represents hydrogen.

In an embodiment, the present invention relates to those compounds of Formula (I) and the pharmaceutically acceptable salts, and the solvates thereof, or any subgroup thereof as mentioned in any of the other embodiments, wherein

R¹ represents C₂₋₆alkyl substituted with one substituent selected from the group consisting of Het¹, —OR³, and —NR^{4a}R^{4b}; and

R² represents hydrogen.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-x):

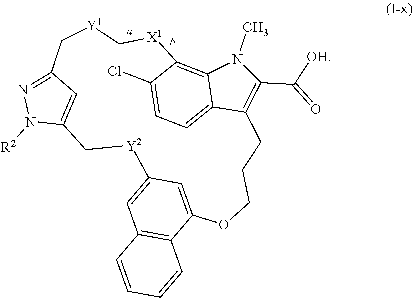

It will be clear that all variables in the structure of Formula (I-x), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-y):

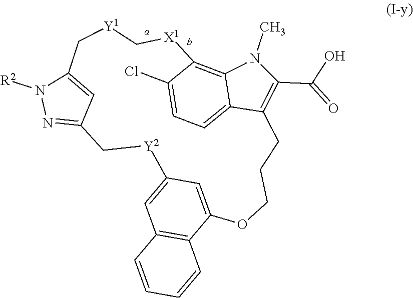

It will be clear that all variables in the structure of Formula (I-y), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds are Rₐ atropisomers.

In an embodiment, the present invention relates to those compounds of Formula (I) and the pharmaceutically acceptable salts, and the solvates thereof, or any subgroup thereof as mentioned in any of the other embodiments, wherein the compounds are Sₐ atropisomers.

In an embodiment, the present invention relates to those compounds of Formula (I) and the pharmaceutically acceptable salts, and the solvates thereof, or any subgroup thereof as mentioned in any of the other embodiments, provided that

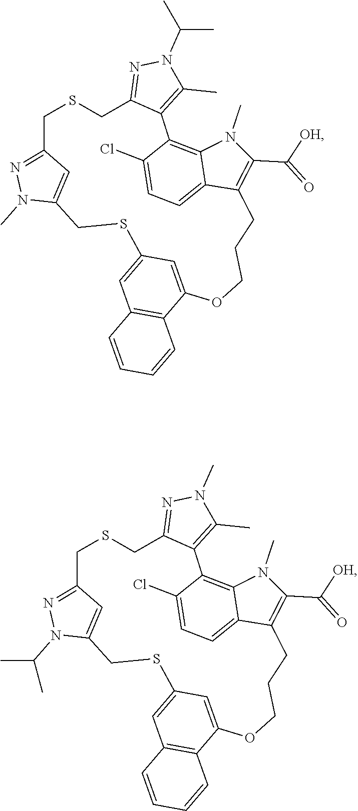 [SMILES: Cc1c2c(nn1C(C)C)CSCc1cc(n(C)n1)CSc1cc(c3ccccc3c1)OCCCc1c(C(=O)O)n(C)c3c-2c(Cl)ccc13 ; Cc1c2c(nn1C)CSCc1cc(n(C(C)C)n1)CSc1cc(c3ccccc3c1)OCCCc1c(C(=O)O)n(C)c3c-2c(Cl)ccc13]

and the tautomers and the stereoisomeric forms thereof are excluded. In an embodiment, the scope of the present invention does not include said excluded compounds, and the pharmaceutically acceptable salts thereof. In an embodiment, the scope of the present invention does not include said excluded compounds, and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, provided that

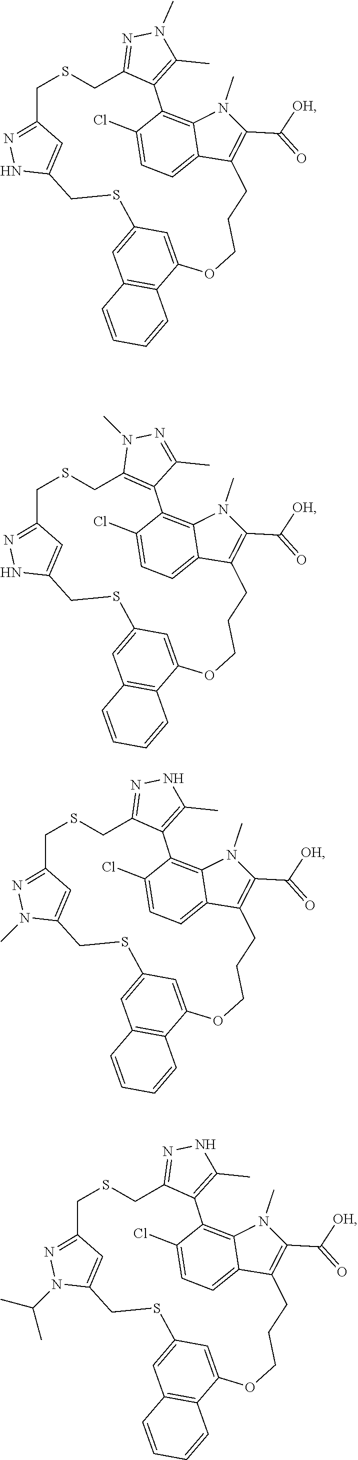

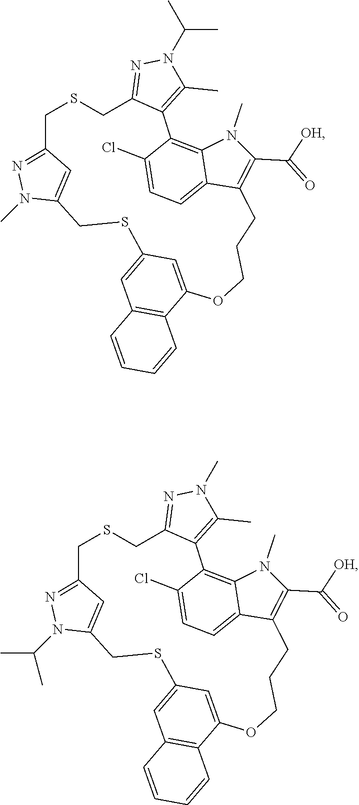 [SMILES: Cc1c2c(nn1C(C)C)CSCc1cc(n(C)n1)CSc1cc(c3ccccc3c1)OCCCc1c(C(=O)O)n(C)c3c-2c(Cl)ccc13 ; Cc1c2c(nn1C)CSCc1cc(n(C(C)C)n1)CSc1cc(c3ccccc3c1)OCCCc1c(C(=O)O)n(C)c3c-2c(Cl)ccc13]

and the tautomers and the stereoisomeric forms thereof are excluded. In an embodiment, the scope of the present invention does not include said excluded compounds, and the pharmaceutically acceptable salts thereof. In an embodiment, the scope of the present invention does not include said excluded compounds, and the pharmaceutically acceptable salts and the solvates thereof.

In an embodiment, the present invention relates to a subgroup of Formula (I) as defined in the general reaction schemes.

In an embodiment the compound of Formula (I) is selected from the group consisting of any of the exemplified compounds,

tautomers and stereoisomeric forms thereof,
any pharmaceutically acceptable salts, and the solvates thereof.

All possible combinations of the above indicated embodiments are considered to be embraced within the scope of the invention.

Methods for the Preparation of Compounds

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or prepared by standard synthetic processes commonly used by those skilled in the art of organic chemistry. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

Compounds of Formula (I), wherein X¹, X², Y¹, and Y² are as defined in the general scope, can be prepared according to Scheme 1. P1 is a suitable protecting group such as, for example, paramethoxybenzyl (PMB) or dimethoxylbenzyl (DMB).

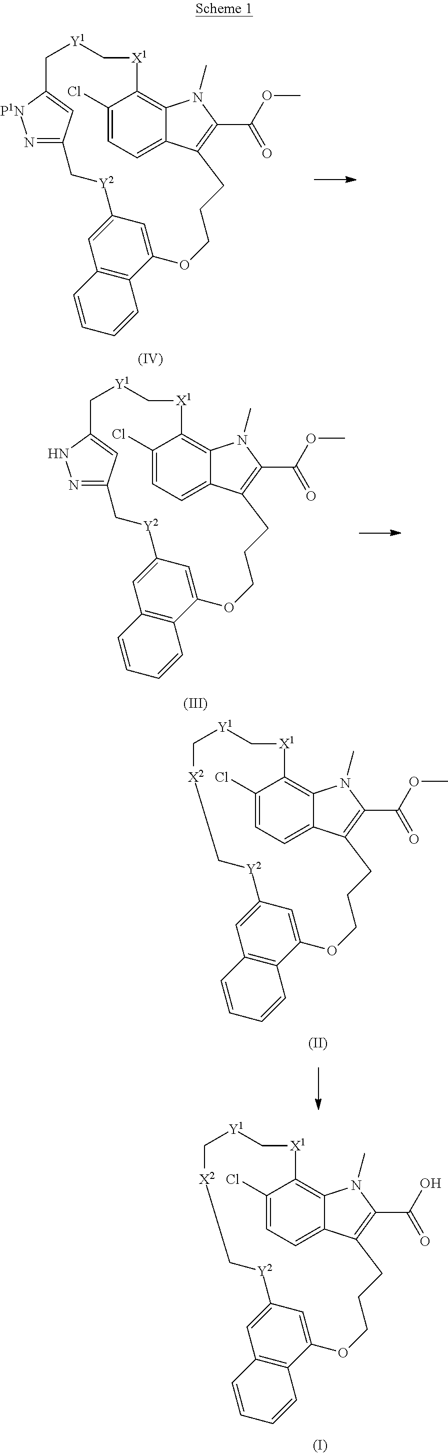

By reacting with an intermediate of Formula (II) with a suitable base, such as, for example, LiOH or NaOH, in a suitable solvent, such as water or a mixture of water and a suitable organic solvent such as dioxane or THE (tetrahydrofuran), or a mixture of MeOH and THF, at a suitable temperature, such as room temperature or 60° C.
Intermediates of Formula (II) can be prepared by reacting an intermediate of Formula (III) with a suitable alkylating agent R²L (where L is as suitable leaving group) such as, for example, an alkyl halide, in the presence of a suitable base such as, for example, Cs₂CO₃, in a suitable solvent such as, for example, DMF, at a suitable temperature such as, for example, room temperature or 60° C.
Intermediates of Formula (III) can be prepared by reacting an intermediate of Formula (IV) with a suitable deprotecting agent such as, for example, HCl, in a suitable solvent such as, for example, MeOH, THF, or a mixture thereof, at a suitable temperature such as, for example, room temperature.

An intermediate of Formula (II) might have a protecting group in the R¹ position, such as for example tetrahydropyranyl. In such a case, the intermediate of Formula (II) is reacted with a suitable deprotecting agent, such as, for example, pTsOH (p-toluenesulfonic acid) or HCl, in a suitable solvent such as, for example, iPrOH (2-propanol), at a suitable temperature such as, for example, room temperature. In a next step the obtained unprotected intermediate can be reacted with a suitable alkylating agent R¹L (where L is as suitable leaving group) such as, for example, an alkyl halide, in the presence of a suitable base such as, for example, Cs₂CO₃, in a suitable solvent such as, for example, DMF (N,N-dimethylformamide), at a suitable temperature such as, for example, room temperature or 60° C.

Alternatively, intermediates of Formula (II), when Y¹=Y²=SO₂, can also be prepared by reacting an intermediate of Formula (II), where Y¹=Y²=S, with a suitable oxidizing agent such as, for example, mCPBA (metachloro perbenzoic acid), in a suitable solvent such as, for example, DCM (dichloromethane), at a suitable temperature such as, for example, room temperature.

Intermediates of Formula (IV), wherein X¹ is as defined in Formula (I), Y² is S, and P¹ is a suitable protecting group such as, for example, paramethoxybenzyl (PMB), or dimethoxylbenzyl (DMB), can be prepared according to Scheme 2,

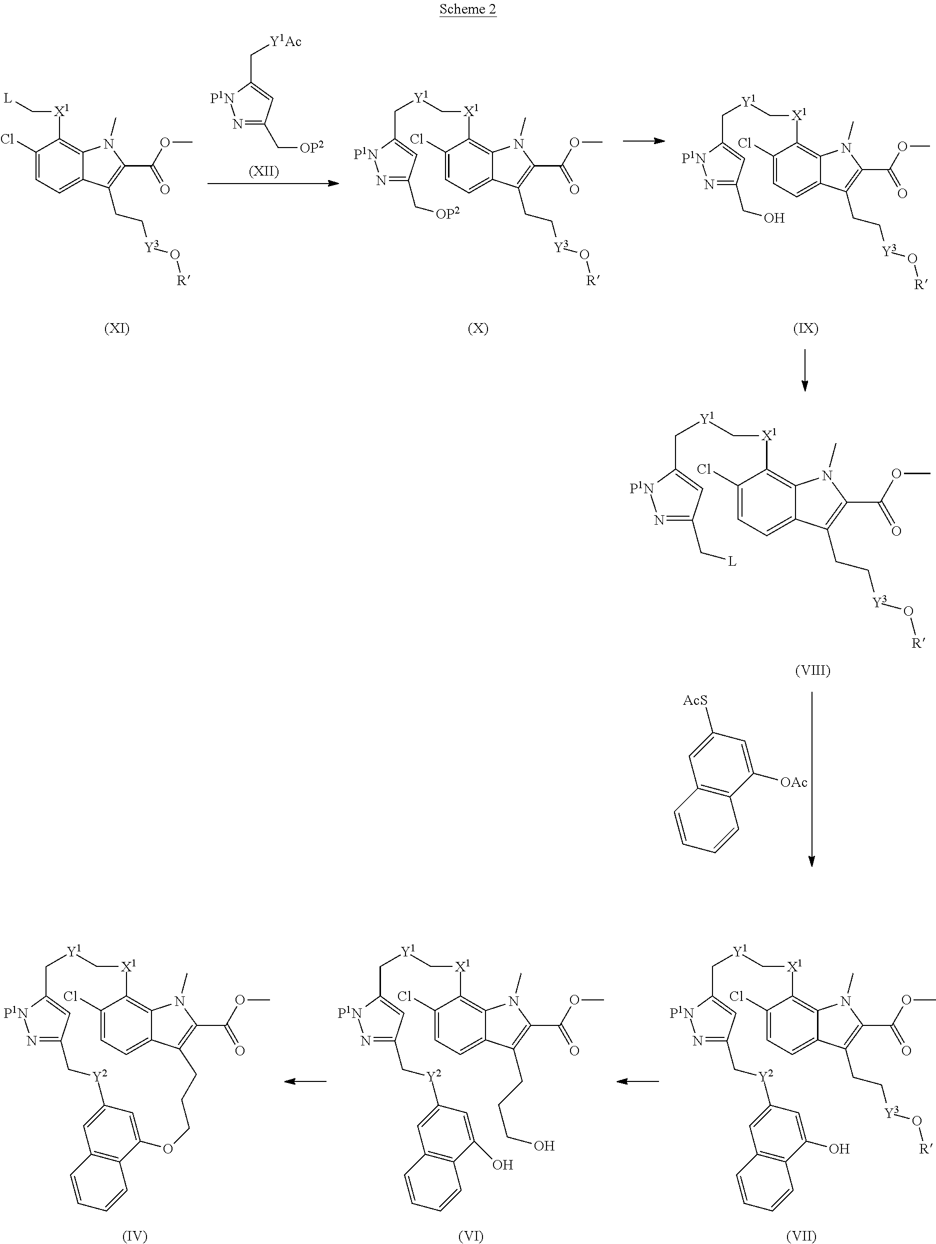

By reacting an intermediate of Formula (VI), with a suitable reagent such as, for example, diethyl azodicarboxylate (DEAD) or di-tert-butyl azodicarboxylate (DTBAD), in the presence of a suitable phosphine such as, for example, PPh₃, in a suitable solvent such as, for example, THF, toluene, or a mixture thereof, at a suitable temperature such as, for example, room temperature or 70° C.
Intermediates of Formula (VI) can be prepared by reacting an intermediate of Formula (VII), wherein Y³ is C═O and R′ is Me, with a suitable reducing agent such as, for example, BH₃.DMS (borane dimethylsulfide), in a suitable solvent such as, for example, THF, at a suitable temperature such as, for example, room temperature or 50° C.
Alternatively, Intermediates of Formula (VI) can be prepared by reacting an intermediate of Formula (VII), wherein Y³ is CH₂ and R′ is a suitable protecting group such as TBDMS, with a suitable deprotecting agent such as, for example, tetrabutylammonium fluoride (TBAF), in a suitable solvent such as, for example, THF, at a suitable temperature such as, for example, room temperature.
Intermediates of Formula (VII) can be prepared by reacting an intermediate of Formula (VIII), where L is a suitable leaving group such as, for example, mesylate (MsO) or Cl, with 3-(acetylthio)naphthalen-1-yl acetate, in the presence of a suitable base, such as, for example, K₂CO₃, in a suitable solvent, such as, for example, methanol, at a suitable temperature, such as, for example, room temperature.
Intermediates of Formula (VIII) can be prepared by reacting an intermediate of Formula (IX), with a suitable activating agent such as, for example, mesyl chloride (MsCl) or SOCl₂, in a suitable solvent such as DCM, at a suitable temperature such as, for example, room temperature.
Intermediates of Formula (IX) can be prepared by:
   a) when Y³ is C═O, R′ is Me, and P² is a protecting group such as TBDMS: reacting an intermediate of Formula (X), with a suitable deprotecting agent such as, for example, TBAF, in a suitable solvent such as, for example, THF, at a suitable temperature such as, for example, room temperature; or
   b) when Y³ is CH₂, R′ is a protecting group such as TBDMS, and P² is a protecting group such as tetrahydropyranyl (THP):
reacting an intermediate of Formula (X), with a suitable deprotecting agent such as, for example, MgBr₂, in a suitable solvent such as, for example Et₂O, at a suitable temperature such as, for example, room temperature.
Intermediates of Formula (X), wherein P² is a suitable protecting group such as, for example, tertbutyldiphenylsilyl (TBDPS), can be prepared by reacting an intermediate of Formula (XI), with an intermediate of Formula (XII), in the presence of a suitable base such as, for example, K₂CO₃, in a suitable solvent such as, for example, MeOH, THF, or a mixture thereof, at a suitable temperature such as, for example, room temperature. L is defined as a suitable leaving group such as for example MsO or Cl.

Alternatively, intermediates of Formula (VI), wherein X¹ and R^{x} are as defined in Formula (I), Y² is S, and P¹ is a suitable protecting group such as, for example, paramethoxybenzyl (PMB), or dimethoxylbenzyl (DMB), can be prepared according to Scheme 3,

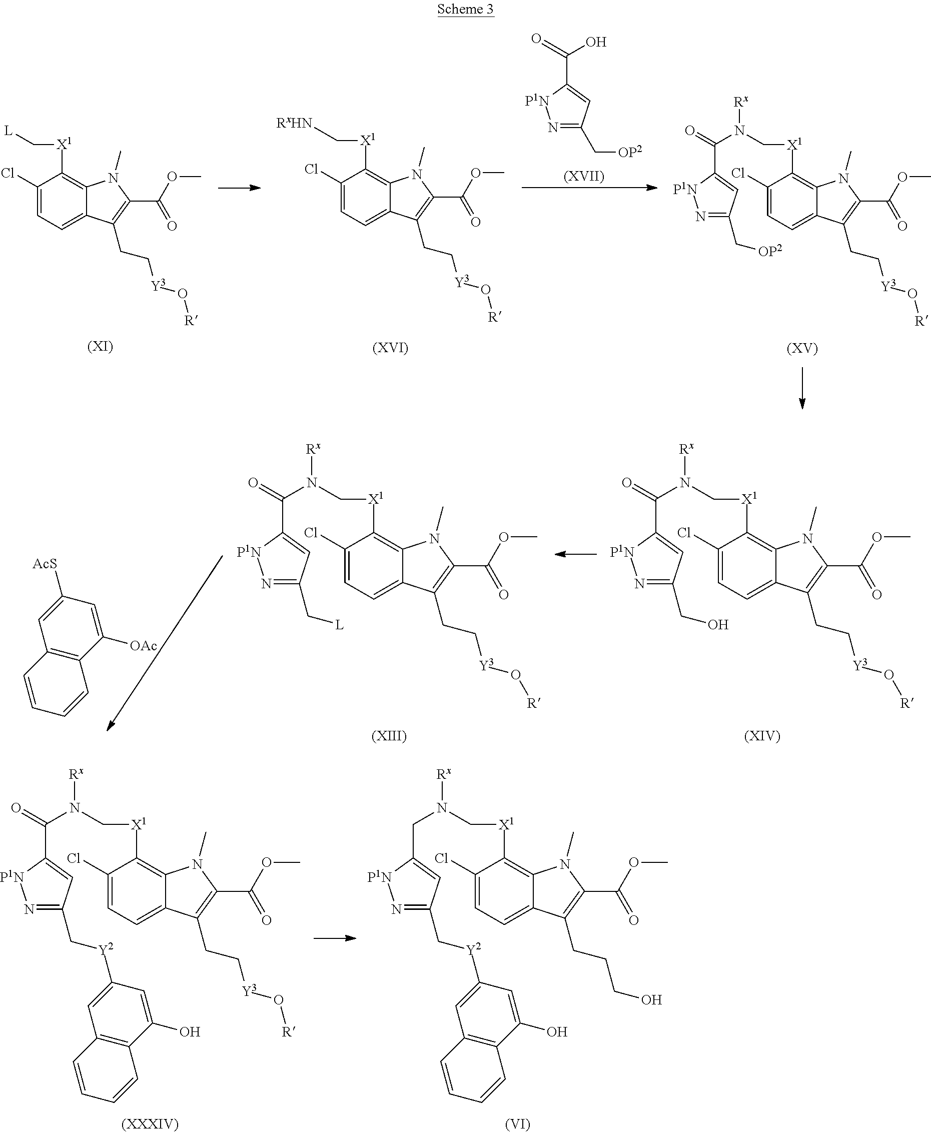

By reacting an intermediate of Formula (XXXIV), wherein Y³ is C═O and R′ is Me, with a suitable reducing agent such as, for example, BH₃.DMS (borane dimethylsulfide), in a suitable solvent such as, for example, THF, at a suitable temperature such as, for example, room temperature or 50° C.
Alternatively, Intermediates (VI) can be prepared in two steps, first by reacting an intermediate of Formula (XXXIV), wherein Y³ is CH₂ and R′ is a suitable protecting group such as TBDMS, with a suitable reducing agent such as, for example, BH₃.DMS (borane dimethylsulfide), in a suitable solvent such as, for example, THF, at a suitable temperature such as, for example, room temperature or 50° C., followed by reacting with a suitable deprotecting agent such as, for example, TBAF, in a suitable solvent such as, for example, THF, at a suitable temperature such as, for example, room temperature.
Intermediates of Formula (XXXIV) can be prepared by reacting an intermediate of Formula (XIII) wherein L is a suitable leaving group such as, for example, MsO or Cl, with 3-(acetylthio)naphthalen-1-yl acetate, in the presence of a suitable base, such as, for example, K₂CO₃, in a suitable solvent, such as, for example, methanol, at a suitable temperature, such as, for example, room temperature.
Intermediates of Formula (XIII) can be prepared by reacting an intermediate of Formula (XIV) with a suitable activating agent such as, for example, MsCl or SOCl₂, in a suitable solvent such as DCM, at a suitable temperature such as, for example, room temperature.
Intermediates of Formula (XIV) can be prepared by reacting an intermediate of Formula (XV), with a suitable deprotecting agent such as, when Y³ is C═O, R′ is Me, and P² is a protecting group such as TBDMS, for example, TBAF, in a suitable solvent such as, for example, THF, at a suitable temperature such as, for example, room temperature; or, when Y³ is CH₂, R′ is a protecting group such as TBDMS, and P² is a protecting group such as THP, for example, MgBr₂, in a suitable solvent such as, for example Et₂O, at a suitable temperature such as, for example, room temperature.
Intermediates of Formula (XV) can be prepared by reacting an intermediate of Formula (XVI) with an intermediate of Formula (XVII), in the presence of a suitable coupling reagent such as, for example, 0-(Benzotriazol-1-yl)-N,N,N′,N′-tetramethyluronium hexafluorophosphate (HBTU), in the presence of a suitable base such as, for example, DIPEA, in a suitable solvent such as, for example, DMF, at a suitable temperature such as, for example, room temperature.
Intermediates of Formula (XVI) can be prepared by reacting an intermediate of Formula (XI) with a suitable primary amine such as, for example, methylamine, in a suitable solvent such as, for example, THF, at a suitable temperature such as, for example, 40° C.

Intermediates of Formula (XVII) wherein P¹ is as defined in (VII) and P² is a suitable protecting group such as, for example, tertbutyldimethylsilyl (TBDMS), can be prepared according to Scheme 4,

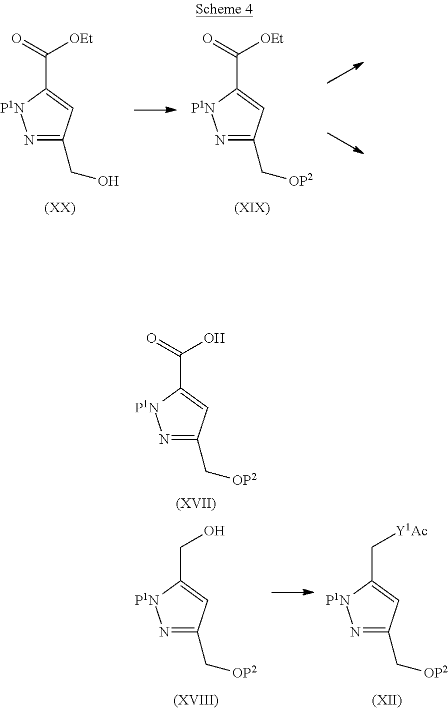

by reacting an intermediate of Formula (XIX) in the presence of a suitable base such as, for example, NaOH, in a suitable solvent such as, for example, a mixture of MeOH and water, at a suitable temperature such as, for example, room temperature.

Intermediates of Formula (XIX), where P² is a protecting group such as, for example, THP, can be prepared according to Scheme 4 by reacting an intermediate of Formula (XX) with a suitable protecting group precursor such as, for example, dihydropyrane, in the presence of a suitable acid such as, for example, paratoluenesulfonic acid (pTosOH), in a suitable solvent such as, for example, DCM, at a suitable temperature such as, for example, room temperature. Intermediates of Formula (XIX), where P² is a protecting group such as, for example, TBDMS, can be prepared according to Scheme 4 by reacting an intermediate of Formula (XX) with a suitable protecting group precursor such as, for example, tert-butyldimethylchlorosilane (TBDMSCl), in the presence of a suitable base such as, for example, Et₃N or 4-dimethylaminopyridine (DMAP), or a mixture thereof, in a suitable solvent such as, for example, THF, at a suitable temperature such as, for example, room temperature. Intermediates of formula (XX) can be prepared by methods known by a person skilled in the art or by analogy to teachings in WO2005018557.

Intermediates of Formula (XII) can be prepared according to Scheme 4,
by reacting an intermediate of Formula (XVIII), in a two-steps procedure, first in the presence of a suitable activating agent such as, for example, MsCl, in the presence of a suitable base such as, for example, Et₃N, in a suitable solvent such as, for example, THF, at a suitable temperature such as, for example, room temperature, then by reacting with potassium thioacetate (AcSK) in a suitable solvent such as, for example, DMF, at a suitable temperature such as, for example, room temperature.
Intermediates of Formula (XVIII) can be prepared by reacting an intermediate of Formula (XIX) with a suitable reducing agent such as, for example, LiAlH₄, in a suitable solvent such as, for example, THF, at a suitable temperature such as, for example, 0° C.

Alternatively, intermediates of Formula (IV) wherein Y¹ is defined as N(R^{x}) can be prepared according to Scheme 5,

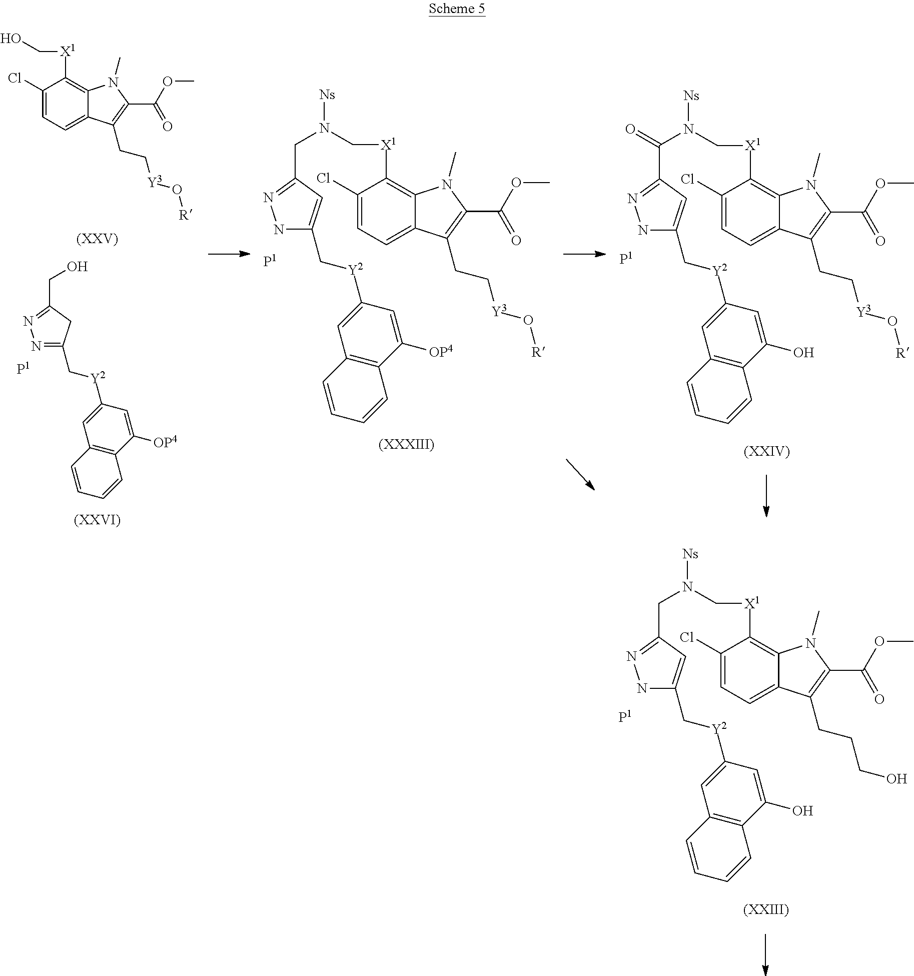

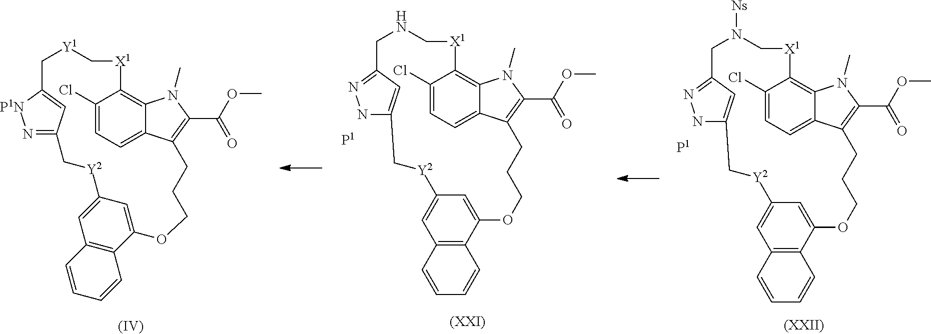

By reacting intermediates of Formula (XXI) with a suitable aldehyde such as, for example, formaldehyde, in the presence of a suitable acid such as, for example, AcOH, in the presence of a suitable reducing agent such as, for example, NaBH(OAc)₃, in a suitable solvent such as, for example, DCM, at a suitable temperature such as, for example, room temperature.
Intermediates of Formula (XXI) can be prepared by reacting an intermediate of Formula (XXII) with a suitable deprotecting agent such as, for example, thiophenol, in the presence of a suitable base such as, for example, K₂CO₃, in a suitable solvent such as, for example, acetonitrile, at a suitable temperature such as, for example, room temperature.
Intermediates of Formula (XXII) can be prepared by reacting an intermediate of Formula (XXIII) with a suitable reagent such as, for example, di-tert-butyl azodicarboxylate (DTBAD), in the presence of a suitable phosphine such as, for example, triphenylphosphine (PPh₃), in a suitable solvent such as, for example, THF, toluene, or a mixture thereof, at a suitable temperature such as, for example, room temperature or 70° C.
Intermediates of Formula (XXIII) can be prepared by reacting an intermediate of Formula (XXIV), wherein Y³ is C═O and R′ is Me, with a suitable reducing agent such as, for example, BH₃.DMS, in a suitable solvent such as, for example, THF, at a suitable temperature such as, for example, room temperature or 50° C.
Alternatively, Intermediates of Formula (XXIII) can also be prepared by reacting an intermediate of Formula (XXXIII), wherein Y³ is CH₂ and R′ is a suitable protecting group such as TBDMS, with a suitable deprotecting agent such as, for example, TBAF in a suitable solvent such as, for example, THF, at a suitable temperature such as, for example, room temperature.
Intermediates of Formula (XXIV) can be prepared by reacting an intermediate of Formula (XXXIII), wherein Y³ is C═O and R′ is Me, with a suitable deprotecting agent such as, for example, TBAF in a suitable solvent such as, for example, THF, at a suitable temperature such as, for example, room temperature.
Intermediates of Formula (XXXIII) can be prepared in a two-step procedure, first by reacting an intermediate of Formula (XXV) with a suitable protected nitrogen such as, for example, 2-nitrophenylsulfonamide, in the presence of a suitable reagent such as, for example, DEAD or DTBAD, in the presence of a suitable phosphine such as, for example, PPh₃, in a suitable solvent such as, for example, DCM, at a suitable temperature such as, for example, room temperature, followed by reacting with an intermediate of Formula (XXVI) in the presence of a suitable reagent such as, for example, DEAD or DTBAD, in the presence of a suitable phosphine such as, for example, PPh₃, in a suitable solvent such as, for example, DCM, at a suitable temperature such as, for example, room temperature. (Ns means nosyl or ortho-nitrobenzenesulfonyl)
Alternatively, intermediates of Formula (XXXIII) may be converted to their oxidized form (wherein Y²=SO₂) by reacting an intermediate of Formula (XXXIII) (wherein Y²=S) with a suitable oxidizing agent such as, for example, mCPBA, in a suitable solvent such as, for example, DCM, at a suitable temperature such as, for example, room temperature.

Intermediates of Formula (XI) wherein X¹ is as defined in Formula (I) and Y³/R′ is C═O/Me or Y³/R′ is CH₂/TBDMS, can be prepared according to Scheme 6,

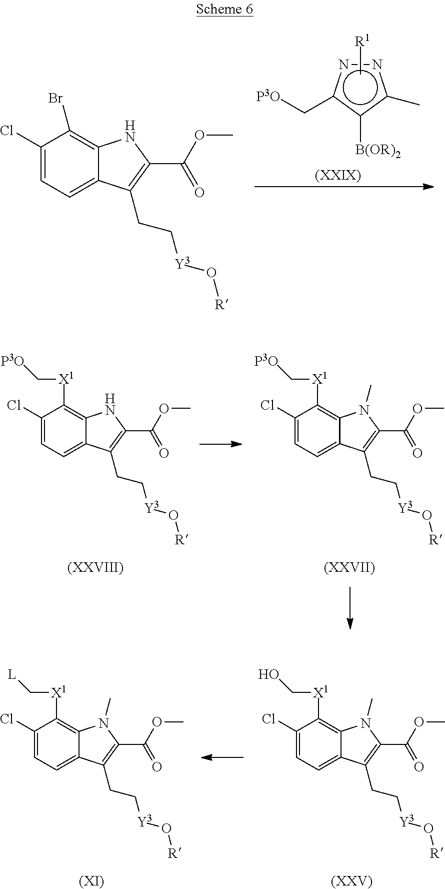

By reacting intermediates of Formula (XXV) with a suitable activating agent such as, for example, MsCl or SOCl₂, in a suitable solvent such as DCM, at a suitable temperature such as, for example, room temperature.
Intermediates of Formula (XXV) can be prepared by reacting an intermediate of Formula (XXVII) with a suitable deprotecting agent such as, for example, TFA, in a suitable solvent such as, for example, DCM, at a suitable temperature such as, for example, room temperature.
Intermediates of Formula (XXVII) can be prepared by reacting an intermediate of Formula (XXVIII) with a suitable alkylating reagent such as, for example, Mel (methyl iodide), in the presence of a suitable base such as, for example, Cs₂CO₃, in a suitable solvent such as, for example, DMF, at a suitable temperature such as, for example, room temperature.
Intermediates of Formula (XXVIII) wherein P³ is a suitable protecting group such as, for example, THP, Y³ is C═O, and R′ is Me, can be prepared by reacting methyl 7-bromo-6-chloro-3-(3-methoxy-3-oxopropyl)-1H-indole-2-carboxylate (CAS [2143010-85-7] with an intermediate of Formula (XXIX), in the presence of a suitable catalyst such as, for example, [1,1′-Bis(di-tert-butylphosphino)ferrocene]dichloropalladium(II) (Pd(dtbpf)Cl₂), in the presence of a suitable base such as, for example, Cs₂CO₃, in a suitable solvent such as, for example, a mixture of THE and water, at a suitable temperature such as, for example, 100° C.
Alternatively, this whole synthetic pathway may start from methyl 7-bromo-6-chloro-3-(3-hydroxypropyl)-1H-indole-2-carboxylate (CAS [2245716-18-9]) after its protection by a suitable protecting group reagent such as, for example, TBDMSCl, in the presence of a suitable base such as, for example, triethylamine (Et₃N) or DMAP, or a mixture thereof, in a suitable solvent such as, for example, THF, at a suitable temperature such as, for example, room temperature, leading to intermediates wherein Y³ is CH₂ and R′ is a suitable protecting group such as TBDMS.

Intermediates of Formula (XXIX) wherein R¹ is as defined in Formula (I) or, alternatively, R¹ may also be a suitable protecting group such as, for example, THP; P³ is a suitable protecting group such as, for example, TBDMS; and B(OR)₂ represents a boronic acid or suitable boronate derivative, can be prepared according to Scheme 7,

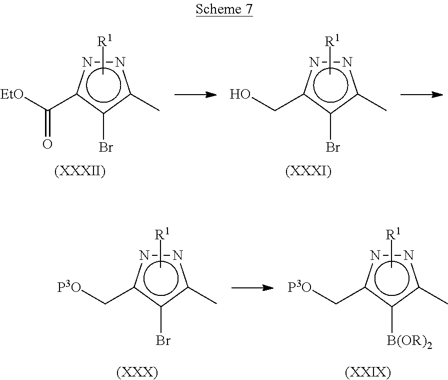

By reacting an intermediate of Formula (XXX) with a suitable boronate such as, for example, isopropoxyboronic acid pinacol ester, in the presence of a suitable base such as, for example, BuLi, in a suitable solvent, such as, for example, THF, at a suitable temperature such as, for example, −78° C.
Intermediates of Formula (XXX) can be prepared by reacting an intermediate of Formula (XXXI) with a suitable protecting group precursor such as, for example, TBDMSCl, in the presence of a suitable base such as, for example, Et₃N or DMAP, or a mixture thereof, in a suitable solvent such as, for example, THF, at a suitable temperature such as, for example, room temperature.
Intermediates of Formula (XXXI) can be prepared by reacting an intermediate of Formula (XXXII) with a suitable reducing agent such as, for example, LiBH₄, in a suitable solvent such as, for example, 2-methyltetrahydrofuran (2-MeTHF), at a suitable temperature such as, for example, room temperature.

Intermediates of Formula (XXVI) can be prepared according to Scheme 8,

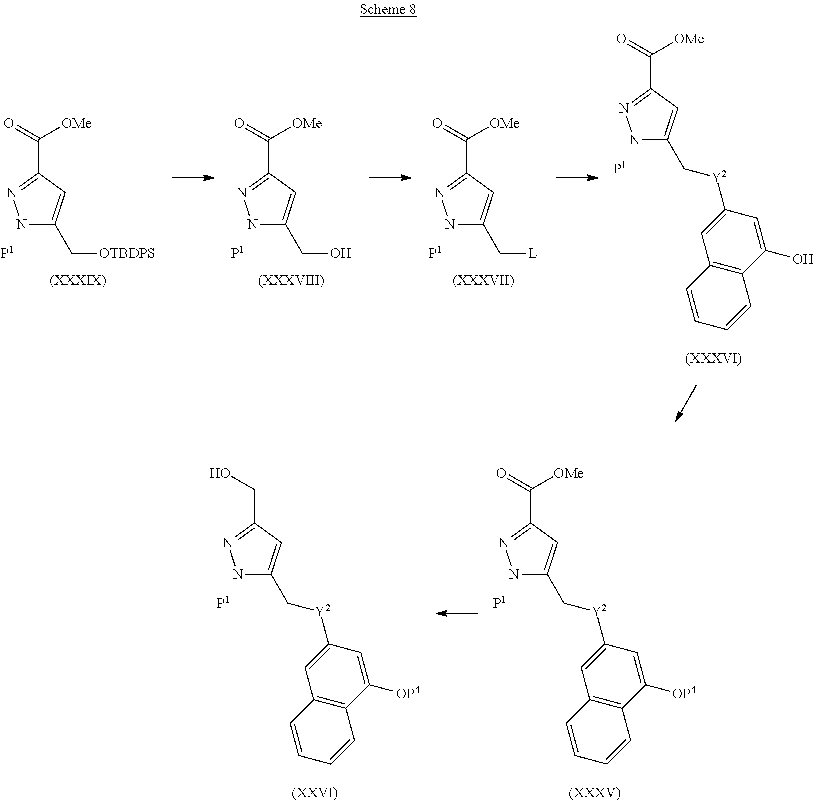

By reacting an intermediate of Formula (XXXV), with a suitable reducing agent such as, for example, DIBALH, in a suitable solvent, such as, for example, THF, at a suitable temperature, such as, for example, 0° C. or room temperature.
Intermediates of Formula (XXXV) can be prepared by reacting an intermediate of Formula (XXXVI), with a suitable trisubstituted silyl chloride such as, for example, TBDMSCl (tert-butyldimethylsilyl chloride) or TBDPSCl (tert-butyldiphenylsilyl chloride), in the presence of a suitable base, such as, for example, imidazole, in a suitable solvent, such as, for example, DMF, at a suitable temperature, such as, for example, room temperature.
Intermediates of Formula (XXXVI) can be prepared by reacting an intermediate of Formula (XXXVII) where L is a suitable leaving group, such as, for example, chloride or mesylate, with 3-(acetylthio)naphthalen-1-yl acetate, in the presence of a suitable base, such as, for example, K₂CO₃, in a suitable solvent, such as, for example, methanol, at a suitable temperature, such as, for example, room temperature.
Intermediates of Formula (XXXVII) can be prepared by reacting an intermediate of Formula (XXXVIII), with a suitable reagent such as, for example, mesyl chloride or thionyl chloride, if necessary in the presence of a suitable base, such as, for example, triethylamine, in a suitable solvent, such as, for example, CH₂Cl₂, at a suitable temperature, such as, for example, 0° C. or room temperature.
Intermediates of Formula (XXXVIII) can be prepared by reacting an intermediate of Formula (XXXIX), with a deprotecting agent, such as, for example, TBAF, in a suitable solvent, such as, for example THF, at a suitable temperature, such as, for example, room temperature.
Intermediates of Formula (XXXIX) can be prepared by reacting ethyl 5-(((tert-butyldiphenylsilyl)oxy)methyl)-1H-pyrazole-3-carboxylate, with a suitable protecting group precursor, such as, for example, paramethoxybenzyl chloride, dimethoxylbenzyl chloride, or also a suitable alkyl halide, such as, for example, methyl iodide (will afford the methylated pyrazole instead of the protected pyrazole), in the presence of suitable base, such as, for example, sodium bis(trimethylsilyl)amide, in a suitable solvent, such as, for example THF, at a suitable temperature, such as, for example, 0° C. or room temperature.
Alternatively, intermediates of Formula (XXXIX) can be prepared by reacting ethyl 5-(((tert-butyldiphenylsilyl)oxy)methyl)-1H-pyrazole-3-carboxylate, with a suitable protecting group precursor, such as, for example, 3,4-dihydro-2H-pyran, in the presence of suitable catalyst, such as, for example, p-toluenesulfonic acid (PTSA), in a suitable solvent, such as, for example tetrahydrofuran (THF) or CH₂Cl₂, at a suitable temperature, such as, for example, 0° C. or room temperature.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of atropoisomers which can be separated from one another following art-known resolution procedures. The atropoisomeric mixtures of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the atropoisomers are liberated therefrom by alkali. An alternative manner of separating the chiral forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl (CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, N.J., 2007.

Pharmacology of Compounds

It has been found that the compounds of the present invention inhibit one of more MCL-1 activities, such as MCL-1 antiapoptotic activity.

An MCL-1 inhibitor is a compound that blocks one or more MCL-1 functions, such as the ability to bind and repress proapoptotic effectors Bak and Bax or BH3 only sensitizers such as Bim, Noxa or Puma.

The compounds of the present invention can inhibit the MCL-1 pro-survival functions. Therefore, the compounds of the present invention may be useful in treating and/or preventing, in particular treating, diseases that are susceptible to the effects of the immune system such as cancer.

In another embodiment of the present invention, the compounds of the present invention exhibit anti-tumoral properties, for example, through immune modulation.

In an embodiment, the present invention is directed to methods for treating and/or preventing a cancer, wherein the cancer is selected from those described herein, comprising administering to a subject in need thereof (preferably a human), a therapeutically effective amount of a compound of Formula (I), or pharmaceutically acceptable salt, or a solvate thereof.

In an embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of acute lymphoblastic leukemia (ALL), acute myeloid leukemia (AML), B cells acute lymphoblastic leukemia, B-cell chronic lymphocytic leukemia (CLL), bladder cancer, breast cancer, chronic lymphocytic leukemia, chronic myeloid leukemia, colon adenocarcinoma, diffuse large B cell lymphoma, esophageal cancer, follicular lymphoma, gastric cancer, head and neck cancer (including, but not limited to head and neck squamous cell carcinoma), hematopoietic cancer, hepatocellular carcinoma, Hodgkin lymphoma, liver cancer, lung cancer (including but not limited to lung adenocarcinoma), lymphoma, medulloblastoma, melanoma, monoclonal gammopathy of undetermined significance, multiple myeloma, myelodysplastic syndromes, myelofibrosis, myeloproliferative neoplasms, ovarian cancer, ovarian clear cell carcinoma, ovarian serous cystadenoma, pancreatic cancer, polycythemia vera, prostate cancer, rectum adenocarcinoma, renal cell carcinoma, smoldering multiple myeloma, T cell acute lymphoblastic leukemia, T cell lymphoma, and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is preferably selected from the group consisting of acute lymphoblastic leukemia (ALL), acute myeloid leukemia (AML), B cells acute lymphoblastic leukemia, B-cell chronic lymphocytic leukemia (CLL), breast cancer, chronic lymphocytic leukemia, chronic myeloid leukemia, diffuse large B cell lymphoma, follicular lymphoma, hematopoietic cancer, Hodgkin lymphoma, lung cancer (including, but not limited to lung adenocarcinoma) lymphoma, monoclonal gammopathy of undetermined significance, multiple myeloma, myelodysplastic syndromes, myelofibrosis, myeloproliferative neoplasms, smoldering multiple myeloma, T cell acute lymphoblastic leukemia, T cell lymphoma and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of adenocarcinoma, benign monoclonal gammopathy, biliary cancer (including, but not limited to, cholangiocarcinoma), bladder cancer, breast cancer (including, but not limited to, adenocarcinoma of the breast, papillary carcinoma of the breast, mammary cancer, medullary carcinoma of the breast), brain cancer (including, but not limited to, meningioma), glioma (including, but not limited to, astrocytoma, oligodendroglioma; medulloblastoma), bronchus cancer, cervical cancer (including, but not limited to, cervical adenocarcinoma), chordoma, choriocarcinoma, colorectal cancer (including, but not limited to, colon cancer, rectal cancer, colorectal adenocarcinoma), epithelial carcinoma, endothelial sarcoma (including, but not limited to, Kaposi's sarcoma, multiple idiopathic hemorrhagic sarcoma), endometrial cancer (including, but not limited to, uterine cancer, uterine sarcoma), esophageal cancer (including, but not limited to, adenocarcinoma of the esophagus, Barrett's adenocarinoma), Ewing sarcoma, gastric cancer (including, but not limited to, stomach adenocarcinoma), gastrointestinal stromal tumor (GIST), head and neck cancer (including, but not limited to, head and neck squamous cell carcinoma), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, kidney cancer (including, but not limited to, nephroblastoma a.k.a. Wilms' tumor, renal cell carcinoma), liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES), ovarian cancer (including, but not limited to, cystadenocarcinoma, ovarian embryonal carcinoma, ovarian adenocarcinoma), pancreatic cancer (including, but not limited to, pancreatic andenocarcinoma, intraductal papillary mucinous neoplasm (IPMN), Islet cell tumors), prostate cancer (including, but not limited to, prostate adenocarcinoma), skin cancer (including, but not limited to, squamous cell carcinoma (SCC), keratoacanthoma (KA), melanoma, basal cell carcinoma (BCC)) and soft tissue sarcoma (e.g. malignant fibrous histiocytoma (MFH), liposarcoma, malignant peripheral nerve sheath tumor (MPNST), chondrosarcoma, fibrosarcoma, myxosarcoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of benign monoclonal gammopathy, breast cancer (including, but not limited to, adenocarcinoma of the breast, papillary carcinoma of the breast, mammary cancer, medullary carcinoma of the breast), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), and prostate cancer (including, but not limited to, prostate adenocarcinoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of prostate, lung, pancreatic, breast, ovarian, cervical, melanoma, B-cell chronic lymphocytic leukemia (CLL), acute myeloid leukemia (AML), and acute lymphoblastic leukemia (ALL).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is multiple myeloma.

The compounds according to the present invention or pharmaceutical compositions comprising said compounds, may also have therapeutic applications in combination with immune modulatory agents, such as inhibitors of the PD1/PDL1 immune checkpoint axis, for example antibodies (or peptides) that bind to and/or inhibit the activity of PD-1 or the activity of PD-L1 and or CTLA-4 or engineered chimeric antigen receptor T cells (CART) targeting tumor associated antigens.

The compounds according to the present invention or pharmaceutical compositions comprising said compounds, may also be combined with radiotherapy or chemotherapeutic agents (including, but not limited to, anti-cancer agents) or any other pharmaceutical agent which is administered to a subject having cancer for the treatment of said subject's cancer or for the treatment or prevention of side effects associated with the treatment of said subject's cancer.

The compounds according to the present invention or pharmaceutical compositions comprising said compounds, may also be combined with other agents that stimulate or enhance the immune response, such as vaccines.

In an embodiment, the present invention is directed to methods for treating and/or preventing a cancer (wherein the cancer is selected from those described herein) comprising administering to a subject in need thereof (preferably a human), a therapeutically effective amount of co-therapy or combination therapy; wherein the co-therapy or combination therapy comprises a compound of Formula (I) of the present invention and one or more anti-cancer agent(s) selected from the group consisting of (a) immune modulatory agent (such as inhibitors of the PD1/PDL1 immune checkpoint axis, for example antibodies (or peptides) that bind to and/or inhibit the activity of PD-1 or the activity of PD-L1 and or CTLA-4); (b) engineered chimeric antigen receptor T cells (CART) targeting tumor associated antigens; (c) radiotherapy; (d) chemotherapy; and (e) agents that stimulate or enhance the immune response, such as vaccines.

The present invention is directed to compounds of Formula (I) and pharmaceutically acceptable salts, and solvates thereof, for use as a medicament.

The present invention is directed to compounds of Formula (I) and pharmaceutically acceptable salts, and solvates thereof, for use in the inhibition of MCL-1 activity.

As used herein, unless otherwise noted, the term “anti-cancer agents” shall encompass “anti-tumor cell growth agents” and “anti-neoplastic agents”.

The present invention is directed to compounds of Formula (I) and pharmaceutically acceptable salts, and solvates thereof, for use in treating and/or preventing diseases (preferably cancers) mentioned above.

The present invention is directed to compounds of Formula (I) and pharmaceutically acceptable salts, and solvates thereof, for treating and/or preventing diseases (preferably cancers) mentioned above.

The present invention is directed to compounds of Formula (I) and pharmaceutically acceptable salts, and solvates thereof, for treating and/or preventing, in particular for treating, a disease, preferably a cancer, as described herein (for example, multiple myeloma).

The present invention is directed to compounds of Formula (I) and pharmaceutically acceptable salts, and solvates thereof, for use in treating and/or preventing, in particular for treating, a disease, preferably a cancer, as described herein (for example, multiple myeloma).

The present invention is directed to compounds of Formula (I) and pharmaceutically acceptable salts, and solvates thereof, for treating and/or preventing, in particular for treating, MCL-1 mediated diseases or conditions, preferably cancer, more preferably a cancer as herein described (for example, multiple myeloma).

The present invention is directed to compounds of Formula (I) and pharmaceutically acceptable salts, and solvates thereof, for use in treating and/or preventing, in particular for use in treating, MCL-1 mediated diseases or conditions, preferably cancer, more preferably a cancer as herein described (for example, multiple myeloma).

The present invention relates to compounds of Formula (I) and pharmaceutically acceptable salts, and solvates thereof, for the manufacture of a medicament.

The present invention relates to compounds of Formula (I) and pharmaceutically acceptable salts, and solvates thereof, for the manufacture of a medicament for the inhibition of MCL-1.

The present invention relates to compounds of Formula (I) and pharmaceutically acceptable salts, and solvates thereof, for the manufacture of a medicament for treating and/or preventing, in particular for treating, a cancer, preferably a cancer as herein described. More particularly, the cancer is a cancer which responds to inhibition of MCL-1 (for example, multiple myeloma).

The present invention is directed to compounds of Formula (I) and pharmaceutically acceptable salts, and solvates thereof, for the manufacture of a medicament for treating and/or preventing, in particular for treating, any one of the disease conditions mentioned hereinbefore.

The present invention is directed to compounds of Formula (I) and pharmaceutically acceptable salts, and solvates thereof, for the manufacture of a medicament for treating and/or preventing any one of the disease conditions mentioned hereinbefore.

The compounds of Formula (I) and pharmaceutically acceptable salts, and solvates thereof, can be administered to subjects, preferably humans, for treating and/or preventing of any one of the diseases mentioned hereinbefore.

In view of the utility of the compounds of Formula (I) and pharmaceutically acceptable salts, and solvates thereof, there is provided a method of treating subjects, preferably mammals such as humans, suffering from any of the diseases mentioned hereinbefore; or a method of slowing the progression of any of the diseases mentioned hereinbefore in subject, humans; or a method of preventing subjects, preferably mammals such as humans, from suffering from any one of the diseases mentioned hereinbefore.

Said methods comprise the administration, i.e. the systemic or topical administration, preferably oral or intravenous administration, more preferably oral administration, of an effective amount of a compound of Formula (I) or a pharmaceutically acceptable salt, or a solvate thereof, to subjects such as humans.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. In an embodiment, a therapeutically effective daily amount may be from about 0.005 mg/kg to 100 mg/kg.

The amount of a compound according to the present invention, also referred to herein as the active ingredient, which is required to achieve a therapeutic effect may vary on case-by-case basis, for example with the specific compound, the route of administration, the age and condition of the recipient, and the particular disorder or disease being treated. The methods of the present invention may also include administering the active ingredient on a regimen of between one and four intakes per day. In these methods of the present invention, the compounds according to the invention are preferably formulated prior to administration.

The present invention also provides compositions for treating and/or preventing the disorders (preferably a cancer as described herein) referred to herein. Said compositions comprise a therapeutically effective amount of a compound of Formula (I), or a pharmaceutically acceptable salt, or a solvate thereof, and a pharmaceutically acceptable carrier or diluent.

While it is possible for the active ingredient (e.g. a compound of the present invention) to be administered alone, it is preferable to administer it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of the present invention may be prepared by any methods well known in the art of pharmacy, for example, using methods such as those described in, for example, Gennaro et al. Remington's Pharmaceutical Sciences (18^{th} ed., Mack Publishing Company, 1990, see especially Part 8: Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, in an embodiment, the present invention is directed to a product comprising, as a first active ingredient a compound according to the invention and as further, as an additional active ingredient one or more anti-cancer agent(s), as a combined preparation for simultaneous, separate or sequential use in the treatment of patients suffering from cancer.

The one or more other anti-cancer agents and the compound according to the present invention may be administered simultaneously (e.g. in separate or unitary compositions) or sequentially, in either order. In an embodiment, the two or more compounds are administered within a period and/or in an amount and/or a manner that is sufficient to ensure that an advantageous or synergistic effect is achieved. It will be appreciated that the preferred method and order of administration and the respective dosage amounts and regimes for each component of the combination will depend on the particular other anti-cancer agent and the compound of the present invention being administered, their route of administration, the particular condition, in particular tumor, being treated and the particular host being treated.

The following examples further illustrate the present invention.

### EXAMPLES

Several methods for preparing the Compounds of this invention are illustrated in the following examples. Unless otherwise noted, all starting materials were obtained from commercial suppliers and used without further purification, or alternatively can be synthesized by a skilled person by using well-known methods.

| Abbreviation | Meaning |
|---|---|
| DCM | dichloromethane |
| ACN | acetonitrile |
| AcOH | acetic acid |
| DTBAD | Di-tert-butyl Azodicarboxylate |
| HBTU | N,N,N′,N′-Tetramethyl-O-(1H-benzotriazol-1-yl)uronium |
| | hexafluorophosphate |
| DIPEA | N,N-diisopropylethylamine |
| Co | Compound |
| Co. No. | Compound Number |
| DMF | N,N-dimethylformamide |
| Me | methyl |
| EtOAc | ethyl acetate |
| eq. | equivalent(s) |
| EtOH | ethanol |
| quant. | quantitative |
| RP | reversed phase |
| DIBALH | di-isobutylaluminiumhydride |
| HPLC | high performance liquid chromatography |
| NaBH(OAc)₃ | sodium triacetoxyborohydride |
| MeOH | methanol |
| SFC | super critical fluid chromatography |
| THF | tetrahydrofuran |
| rac | racemic |
| Et₃N or TEA | trietylamine |
| Celite ® | diatomaceous earth |
| Pd(dtbpf)Cl₂ | 1,1′-Bis (di-t-butylphosphino)ferrocene palladium |
| | dichloride |
| PPh₃ | triphenylphosphine |
| BuLi | n-butyllithium |
| mCPBA | meta-chloroperoxybenzoic acid |
| Me-THF or | 2-methyltetrahydrofuran |
| 2-Me-THF | |
| iPrOH | isopropanol |
| iPrNH₂ | isopropylamine |
| TBAF | tetrabutylammonium fluoride |
| DMAP | 4-dimethylaminopyridine |
| TBDMSCl | tert-butyldimethylsilyl chloride |
| MsCl | methanesulfonyl chloride |
| SFC | supercritical fluid chromatography |

As understood by a person skilled in the art, Compounds synthesized using the protocols as indicated may contain residual solvent or minor impurities.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

Preparation of Intermediates

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

Intermediate 1

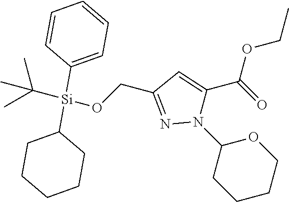 [SMILES: CCOC(=O)c1cc(CO[Si](c2ccccc2)(C2CCCCC2)C(C)(C)C)nn1C1CCCCO1]

Ethyl 3-(hydroxymethyl)-1-(tetrahydro-2H-pyran-2-yl)-1H-pyrazole-5-carboxylate [847139-28-0] (5.2 g, 20.449 mmol) was dissolved in dry DMF (60 mL) under nitrogen atmosphere. Imidazole (2.088 g, 1.5 eq.) and DMAP (250 mg, 0.1 eq.) were added. Tert-butylchlorodiphenylsilane (6.91 mL, 1.3 eq.) was added slowly and the reaction was stirred at room temperature overnight. The mixture was diluted with EtOAc (250 mL) and water (200 mL). The organic layer was separated and washed with brine (3×100 mL). The combined aqueous layers were extracted with EtOAc (150 mL). The combined organic layer was dried over MgSO₄, filtered and evaporated. The residue was purified by flash chromatography on silica gel (120 g, gradient: from heptane 100% up to heptane/EtOAc 8/2). Intermediate 1 (11.56 g, 97% yield) was obtained as a colorless paste.

Intermediate 2

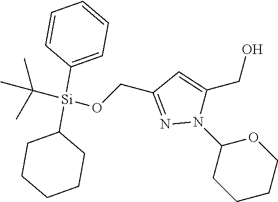 [SMILES: CC(C)(C)[Si](OCc1cc(CO)n(C2CCCCO2)n1)(c1ccccc1)C1CCCCC1]

LiAlH₄ (2 M in THF, 10.97 mL, 1.1 eq) was added dropwise to a solution of Intermediate 1 (9.826 g, 19.94 mmol) in dry THE (80 mL) stirring at 0° C. under nitrogen atmosphere. After stirring at 0° C. for 15 min, the reaction was treated with wet THF (25 mL), then with water (5 mL, added dropwise) and then allowed to warm up to room temperature. Celite was added, followed by MgSO₄ and EtOAc. After 5 min stirring, the suspension was filtered, and the solid was washed with EtOAc. The filtrate was concentrated under reduced pressure. The residue was purified by flash chromatography on silica gel (120 g, gradient: from heptane 100% up to heptane/EtOAc 1/1) to give Intermediate 2 (8.54 g, 95% yield) as a colorless paste.

Intermediate 3

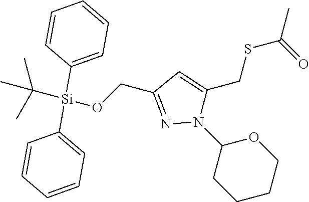 [SMILES: CC(=O)SCc1cc(CO[Si](c2ccccc2)(c2ccccc2)C(C)(C)C)nn1C1CCCCO1]

MsCl (1.84 mL, 1.25 eq.) was added dropwise to a solution of Intermediate 2 (8.54 g, 18.95 mmol) and TEA (3.95 mL, 1.5 eq.) in THE (85 mL) stirring at 0° C. under nitrogen atmosphere. The reaction was then allowed to warm up to room temperature (a solid precipitated) and was stirred at room temperature for 1 h. Potassium thioacetate (3.25 g, 1.5 eq.) dissolved in dry DMF (85 mL) was added and stirring was continued at room temperature for 3 h. The reaction mixture was diluted with EtOAc (250 mL) and water (200 mL). The aqueous layer was separated and the organic one was washed with brine (3×150 mL). The combined aqueous layer was back-extracted with EtOAc (200 mL). The combined organic layer was dried over MgSO₄, filtered and evaporated. The residue was purified by flash chromatography on silica gel (120 g, gradient: from heptane 100% up to heptane/EtOAc 8/2) to afford Intermediate 3 (9.78 g, 91% yield) as a yellow paste.

Intermediate 4

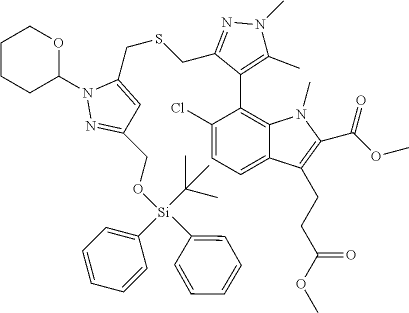 [SMILES: COC(=O)CCc1c(C(=O)OC)n(C)c2c(-c3c(CSCc4cc(CO[Si](c5ccccc5)(c5ccccc5)C(C)(C)C)nn4C4CCCCO4)nn(C)c3C)c(Cl)ccc12]

Methyl 6-chloro-7-(3-(iodomethyl)-1,5-dimethyl-1H-pyrazol-4-yl)-3-(3-methoxy-3-oxopropyl)-1-methyl-1H-indole-2-carboxylate [2143011-01-0] (2.35 g, 4.32 mmol) was dissolved in dry THE (6 mL) and dry MeOH (8 mL) and K₂CO₃ (612 mg, 1.02 eq.) was added. The reaction mixture was degassed with nitrogen. Then, Intermediate 3 (2.42 g, 1.1 eq.) was added dropwise as a solution in dry, degassed, MeOH (8 mL). The reaction was stirred at room temperature for 45 min. The reaction mixture was concentrated under reduced pressure. The residue was partitioned between EtOAc (50 ml) and water (25 mL). The organic layer was separated and the aqueous one was extracted with EtOAc (25 mL). The combined organic layer was dried over MgSO₄, filtered, and evaporated. The residue was purified by flash chromatography on silica gel (80 g, gradient: from heptane 100% up to heptane/EtOAc 1/1) to give Intermediate 4 (2.5 g, 65% yield) as a colorless paste.

Intermediate 5

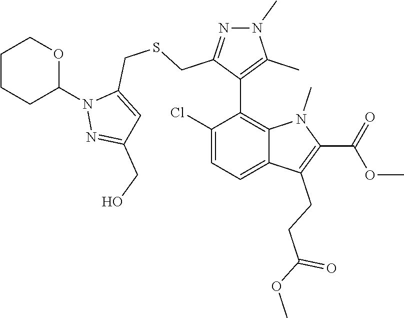 [SMILES: COC(=O)CCc1c(C(=O)OC)n(C)c2c(-c3c(CSCc4cc(CO)nn4C4CCCCO4)nn(C)c3C)c(Cl)ccc12]

TBAF (1 M in THF, 4.53 mL, 1.6 eq.) was added to a solution of Intermediate 4 (2.5 g, 2.83 mmol) in dry THE (55 mL) stirring under nitrogen atmosphere at room temperature. The reaction was stirred at room temperature for 2 h. Volatiles were removed under reduced pressure. The residue was dissolved in EtOAc (150 mL), washed with water (50 mL) and brine (50 mL), dried over MgSO₄, filtered, and evaporated. The residue was purified by flash chromatography on silica gel (80 g, gradient: from DCM 100% up to DCM/MeOH 95/5) to give Intermediate 5 (1.66 g, 91% yield) as a white foamy solid.

Intermediate 6

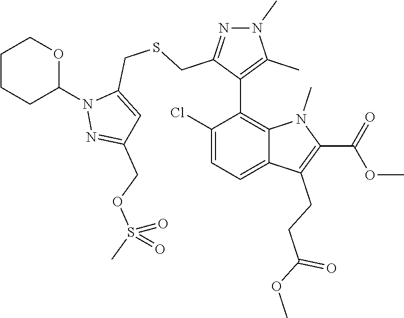 [SMILES: COC(=O)CCc1c(C(=O)OC)n(C)c2c(-c3c(CSCc4cc(COS(C)(=O)=O)nn4C4CCCCO4)nn(C)c3C)c(Cl)ccc12]

MsCl (0.5 mL, 2.5 eq.) was added dropwise to a solution of Intermediate 5 (1.665 g, 2.58 mmol) and TEA (1.08 mL, 3 eq.) in DCM (30 mL), stirring at 0° C. under nitrogen atmosphere. The reaction was then allowed to warm up to room temperature. The reaction was diluted with DCM (20 mL) and treated with saturated aqueous NaHCO₃ (10 mL). The organic layer was separated and the aqueous one was extracted with DCM (10 mL). The combined organic layer was dried over MgSO₄, filtered, and evaporated to give Intermediate 6, used in the next step without further purification.

Intermediate 7

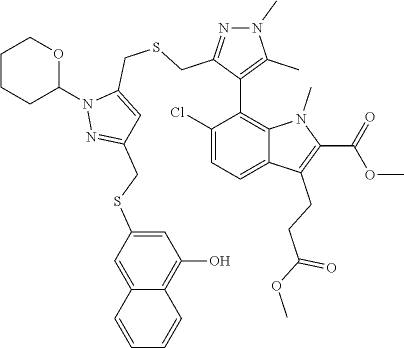 [SMILES: COC(=O)CCc1c(C(=O)OC)n(C)c2c(-c3c(CSCc4cc(CSc5cc(O)c6ccccc6c5)nn4C4CCCCO4)nn(C)c3C)c(Cl)ccc12]

K₂CO₃ (536 mg, 1.5 eq.) was added to a solution of Intermediate 6 (1.867 g, 2.58 mmol) and 3-(acetylthio)naphthalen-1-yl acetate [2143010-96-0] in degassed MeOH at room temperature. The reaction was stirred at room temperature for 2 h. Volatiles were evaporated and the residue was dissolved in EtOAc (100 mL) and water (50 mL). The organic layer was separated and the aqueous one was extracted with EtOAc (50 mL). The combined organic layer was dried over MgSO₄, filtered, and evaporated. The residue was purified by flash column chromatography on silica gel (80 g, gradient: from heptane 100% up to heptane/EtOAc 2/8) to afford Intermediate 7 (1.66 g, 72% yield over 2 steps) as a foamy solid.

Intermediate 8

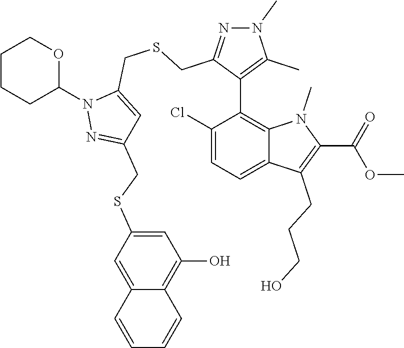 [SMILES: COC(=O)c1c(CCCO)c2ccc(Cl)c(-c3c(CSCc4cc(CSc5cc(O)c6ccccc6c5)nn4C4CCCCO4)nn(C)c3C)c2n1C]

Intermediate 7 (1.66 g, 1.86 mmol) was dissolved in dry THE under nitrogen atmosphere. Borane dimethylsulfide complex (2 M in THF, 4.65 mL, 5 eq.) was added and the reaction was heated to 50° C. for 4 h. The reaction mixture was poured slowly into saturated aqueous NaHCO₃ (50 mL) and MeOH (30 mL) was added. The biphasic mixture was vigorously stirred at room temperature overnight. The mixture was then diluted with EtOAc (100 mL) and water (50 mL). The organic layer was separated and the aqueous one was extracted with EtOAc (2×50 mL). The combined organic layer was dried over MgSO₄, filtered, and evaporated. The residue was purified by flash column chromatography on silica gel (120 g, gradient: from DCM 100% up to DCM/MeOH 97/3) to give Intermediate 8 (1.11 g, 77% yield) as a foamy solid.

Intermediate 9

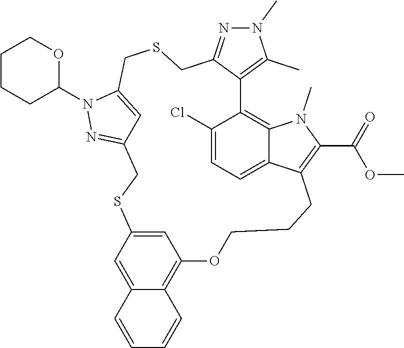 [SMILES: COC(=O)c1c2c3ccc(Cl)c(c3n1C)-c1c(nn(C)c1C)CSCc1cc(nn1C1CCCCO1)CSc1cc(c3ccccc3c1)OCCC2]

A solution of Intermediate 8 (400 mg, 0.51 mmol) and DTBAD (475 mg, 4 eq.) in toluene (20 mL) and THE (1.6 mL) was added via a syringe pump (0.1 mL/min) to a solution of triphenylphosphine (542 mg, 4 eq.) dissolved in toluene (20 mL), while stirring at 70° C. under nitrogen atmosphere. Once the addition was complete, the reaction was allowed to cool to room temperature. The reaction mixture was concentrated under reduced pressure and the residue was purified by flash chromatography on silica gel (40 g, gradient: from DCM 100% up to DCM/MeOH 98/2), to give Intermediate 9 (330 mg, 84% yield) as a white solid.

Intermediate 10

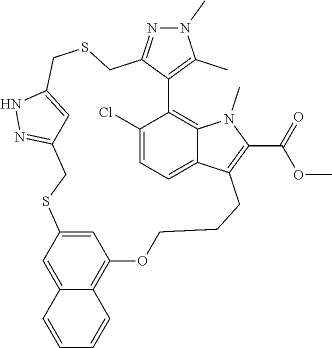 [SMILES: COC(=O)c1c2c3ccc(Cl)c(c3n1C)-c1c(nn(C)c1C)CSCc1cc(n[nH]1)CSc1cc(c3ccccc3c1)OCCC2]

A solution of HCl in MeOH (1.25 M, 17.45 mL, 50 eq.) was added to a solution of Intermediate 9 (330 mg, 0.44 mmol) in dry THF. After 90 min of stirring at room temperature, volatiles were removed under vacuum and the residue was purified by flash column chromatography on silica gel (40 g, gradient: from DCM 100% up to DCM/MeOH 95/5) to afford Intermediate 10 (272 mg, 93% yield) as a white solid. Intermediate 11 and Intermediate 12

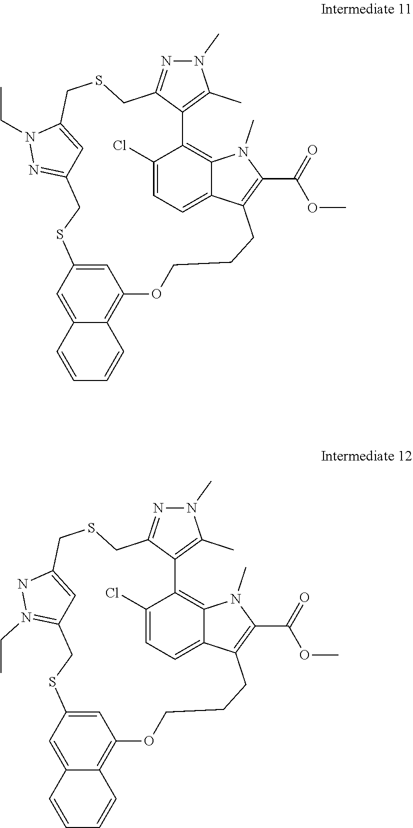

Iodoethane (31 μL, 2.5 eq.) was added to a solution of Intermediate 10 (105 mg, 0.16 mmol) and Cs₂CO₃ (153 mg, 3 eq.) in DMF under nitrogen atmosphere. The reaction was stirred at room temperature for 4 h. The reaction mixture was diluted with EtOAc (20 mL) and water (10 mL). The organic layer was separated and washed with brine (2×10 mL). The combined aqueous layer was back-extracted with EtOAc (10 mL). The combined organic layer was dried over MgSO₄, filtered and evaporated. The residue was purified by flash column chromatography on silica gel (40 g, gradient: from DCM 100% up to DCM/MeOH 97/3) to give Intermediate 11 (61 mg, 49% yield) and Intermediate 12 (59 mg, 49% yield).

Intermediate 13

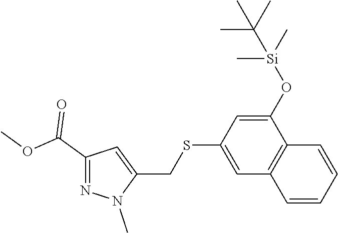 [SMILES: COC(=O)c1cc(CSc2cc(O[Si](C)(C)C(C)(C)C)c3ccccc3c2)n(C)n1]

Imidazole (258 mg, 1.4 eq.) was added to a solution of methyl 5-(((4-hydroxynaphthalen-2-yl)thio)methyl)-1-methyl-1H-pyrazole-3-carboxylate [2245716-34-9] (890 mg, 2.71 mmol) and TBDMSCl (511 mg, 1.25 eq.) in dry DMF (18 mL). The reaction mixture was stirred at room temperature for 48 h. The reaction mixture was diluted with EtOAc (100 mL) and water (50 mL). The organic layer was separated and washed with brine (2×50 mL). The combined aqueous layer was extracted with EtOAc (50 mL). The combined organic layer was dried over MgSO₄, filtered and evaporated. The residue was purified by flash chromatography on silica gel (40 g, gradient: from heptane 100% up to heptane/EtOAc 6/4) to obtain Intermediate 13 (1.24 g, quant.).

Intermediate 14

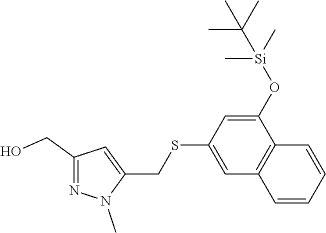 [SMILES: Cn1nc(CO)cc1CSc1cc(O[Si](C)(C)C(C)(C)C)c2ccccc2c1]

DIBALH (1 M in heptane, 5.82 mL, 2.5 eq.) was added dropwise to a solution of Intermediate 13 (1.03 g, 2.33 mmol) in THE (40 mL) at 0° C. under nitrogen atmosphere and the reaction mixture was stirred at 0° C. for 30 min. Additional DIBALH (1 M in heptane, 2.32 mL, 1 eq) was added and stirring was continued at 0° C. for 10 min. The reaction mixture was treated with wet THE (40 mL) and, after a few min stirring, with water (10 mL, initial dropwise addition). The mixture was allowed to warm up to room temperature and then celite was added. After 5 min stirring, the mixture was filtered. The solid was washed with EtOAc. The filtrate was treated with MgSO₄, filtered, and evaporated to give Intermediate 14 (892 mg, 92%) as a colorless paste that solidified upon standing, and was used without further purification.

Intermediate 15

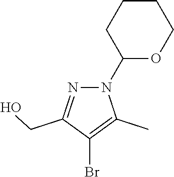

Ethyl 4-bromo-5-methyl-1-(tetrahydro-2H-pyran-2-yl)-1H-pyrazole-3-carboxylate [2246368-58-9] (15.35 g, 48.39 mmol) was dissolved in dry 2-Me-THF (200 mL) and cooled to 0° C. LiBH₄ (4 M in THF, 48.39 mL, 4 eq.) was added slowly and the reaction mixture was allowed to warm to room temperature and was stirred at this temperature overnight. The reaction was quenched with water. The water layer was extracted with EtOAc (3×). The combined organic layer was washed with brine, dried with Na₂SO₄, filtered, and solvents were evaporated to afford Intermediate 15 (12.83 g, 96% yield) as a white powder.

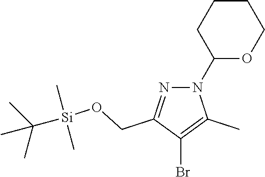 [SMILES: Cc1c(Br)c(CO[Si](C)(C)C(C)(C)C)nn1C1CCCCO1]

Intermediate 16

To a solution of Intermediate 15 (200 mg, 0.73 mmol) in dry THE (5 mL) under nitrogen atmosphere was added DMAP (35 mg, 0.4 eq.) and Et₃N (0.2 mL, 2 eq.) at room temperature. Then, TBDMSCl (115 mg, 1.05 eq.) was added. To allow full conversion, more TBDMSCl (109 mg, 1 eq.) and Et₃N (0.1 mL, 1 eq.) were added to the reaction mixture and it was stirred for another hour. NaHCO₃ and DCM were added to the reaction mixture. The layers were separated and the aqueous layer was extracted twice with DCM. The combined organic layer was washed with brine, dried with Na₂SO₄, filtered, and evaporated. The residue was purified by flash chromatography [Biotage Isolera 1//Biotage SnapUltra Silica 25 g//EtOAc/Heptane. 0/100 to 40/60] to afford Intermediate 16 (238 mg, 84% yield) as a colorless oil.

Intermediate 17

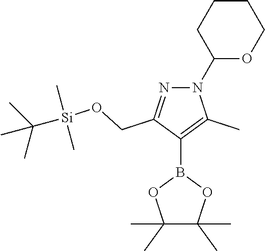 [SMILES: Cc1c(B2OC(C)(C)C(C)(C)O2)c(CO[Si](C)(C)C(C)(C)C)nn1C1CCCCO1]

A solution of Intermediate 16 (5 g, 12.84 mmol) in THF (50 mL) was cooled to −78° C. under nitrogen atmosphere. BuLi (2.5 M in hexane, 7.19 mL, 1.4 eq.) was added dropwise and the mixture was stirred at −78° C. for 20 min. 2-isopropoxy-4,4,5,5-tetramethyl-1,3,2-dioxaborolane [61676-62-8] (3.93 mL, 1.5 eq.) was then added and the reaction was allowed to warm to room temperature. After 15 min at room temperature, the reaction was quenched with water and diluted with DCM. The layers were separated. The aqueous layer was extracted with DCM (3×). The combined organic layer was washed with brine, dried with Na₂SO₄, filtered, and evaporated to afford Intermediate 17 (6.01 g, 62% yield) as a colorless oil used without further purification.

Intermediate 18

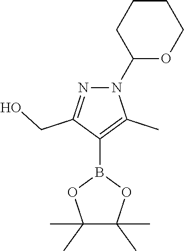 [SMILES: Cc1c(B2OC(C)(C)C(C)(C)O2)c(CO)nn1C1CCCCO1]

To a solution of Intermediate 17 (6.01 g, 7.99 mmol) in dry Me-THF (50 mL) was slowly added TBAF (1 M in THF, 9.58 mL, 1.2 eq.) under nitrogen atmosphere. The reaction mixture was stirred for 15 h. The reaction mixture was diluted with EtOAc, washed with a saturated aqueous NaHCO₃ solution, then with brine, and the combined organic layer was dried with Na₂SO₄, and evaporated. The residue was purified by flash chromatography [Biotage Isolera 1//Biotage SnapUltra Silica 100 g//heptane-EtOAc 100/0 to 80/20] to afford Intermediate 18 (2.43 g, 94% yield) as a white powder.

Intermediate 19

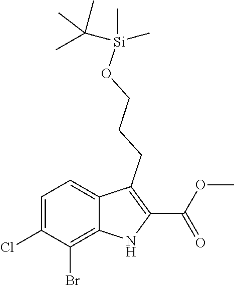 [SMILES: COC(=O)c1[nH]c2c(Br)c(Cl)ccc2c1CCCO[Si](C)(C)C(C)(C)C]

Tert-butyldimethylsilyl chloride (2.06 g, 1.4 eq.) was added portionwise to a mixture of methyl 7-bromo-6-chloro-3-(3-hydroxypropyl)-1H-indole-2-carboxylate [2245716-18-9] (3.5 g, 9.78 mmol) and imidazole (1 g, 1.5 eq.) in DCM (80 mL) at 0° C. DMAP (59 mg, 0.05 eq.) was then added and the reaction mixture was stirred at room temperature for 1 h. The reaction mixture was diluted with DCM and washed with water. The organic layer was separated, dried on MgSO₄, filtered, and evaporated to give Intermediate 19 (4.46 g, 87% yield), used without further purification.

Intermediate 20

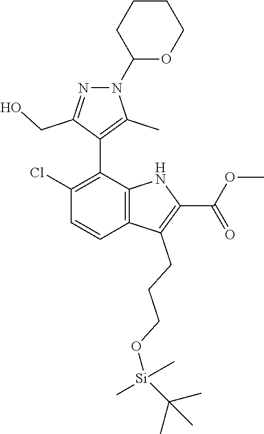 [SMILES: COC(=O)c1[nH]c2c(-c3c(CO)nn(C4CCCCO4)c3C)c(Cl)ccc2c1CCCO[Si](C)(C)C(C)(C)C]

Intermediate 19 (4.85 g, 10.52 mmol), Intermediate 18 (4.07 g, 1.2 eq.), and Cs₂CO₃ (6.85 g, 2 eq.) were dissolved in THE (60 mL) and water (20 mL). This solution was distributed among 5 microwave tubes. These solutions were degassed with nitrogen for 10 min. Pd(dtbpf)Cl₂ (5×41 mg, 0.03 eq.) was then added to each tube and they were sealed and heated to 100° C. in a microwave oven for 30 min. The 5 tubes were combined. Water and EtOAc were added. The layers were separated and the aqueous layer was extracted with EtOAc. The combined organic layer was dried over MgSO₄, filtered, and evaporated. The residue was purified by flash chromatography [Biotage Isolera 1//Biotage SnapUltra Silica 100 g//EtOAc/Heptane:20/80 to 60/40] to afford Intermediate 20 (4.64 g, 76% yield).

Intermediate 21

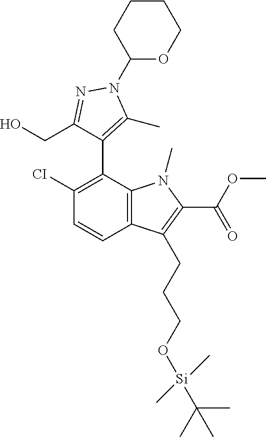

Cs₂CO₃ (2.89 g, 1.1 eq.) was added to a solution of Intermediate 20 (4.64 g, 8.05 mmol) in DMF (45 mL) and the mixture was stirred for 30 min at room temperature. Then, iodomethane (1 mL, 2 eq.) was added to the reaction mixture and it was stirred at room temperature for 1.5 h. Water and EtOAc were added and the layers were separated. The aqueous layer was extracted twice with EtOAc. The organic layer was washed with brine (3×), dried with MgSO₄, filtered, and evaporated to afford Intermediate 21 (4.83 g, quantitative yield), used without further purification.

Intermediate 22

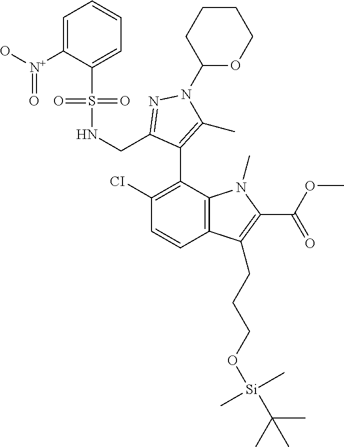

A solution of DTBAD (3.77 g, 2 eq.) in DCM (25 mL) was added dropwise via a syringe pump over 20 min to a suspension of Intermediate 21 (4.83 g, 8.18 mmol), 2-nitrobenzenesulfonamide (1.82 g, 1.1 eq.) and triphenylphosphine (4.29 g, 2 eq.) in DCM (75 mL), stirring at room temperature under nitrogen atmosphere. After full addition, the reaction mixture was concentrated under reduced pressure. The residue was purified by column chromatography on silica gel (100 g, gradient: from heptane 100% up to heptane/EtOAc 4/6) to afford Intermediate 22 (5.85 g, 92% yield).

Intermediate 23

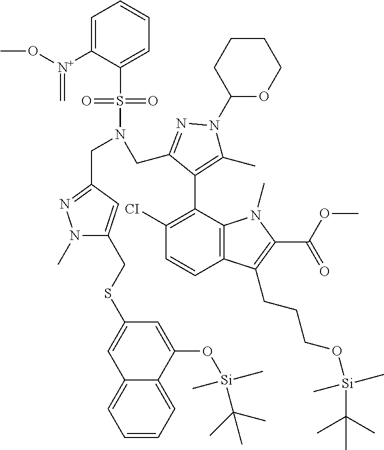

A solution of DTBAD (1.96 g, 2 eq.) in DCM (50 mL) was added dropwise to a suspension of Intermediate 22 (3.29 g, 4.25 mmol), Intermediate 14 (1.94 g, 1.1 eq.) and triphenylphosphine (2.23 g, 2 eq.) in DCM (50 mL) while stirring at room temperature under nitrogen atmosphere. After the addition, the reaction mixture was stirred at room temperature overnight. The solvent was evaporated and the residue was purified by flash chromatography on silica gel (40 g, gradient: from heptane 100% up to heptane/EtOAc 4/6) to afford Intermediate 23 (5.3 g, 91% yield).

Intermediate 24

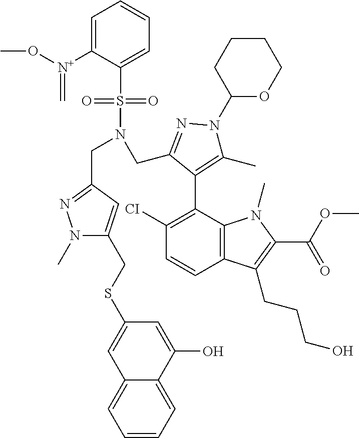

TBAF (1 M in THF, 15.1 mL, 2.5 eq.) was added dropwise to a solution of Intermediate 23 (8.25 g, 6.06 mmol) in THE (200 mL) at room temperature under nitrogen atmosphere.

The reaction mixture was stirred at room temperature for 30 min. The reaction mixture was diluted with DCM and aqueous NH₄Cl was added. Layers were separated. The aqueous layer was extracted 3 times with DCM. The combined organic layer was dried with MgSO₄, filtered, and evaporated. The residue was purified by column flash chromatography [Biotage Isolera 1//Biotage SnapUltra Silica 100 g//EtOAc/Heptane:0/100 to 100/0] to afford Intermediate 24 (5.3 g, 84% yield) as a yellow solid.

Intermediate 25

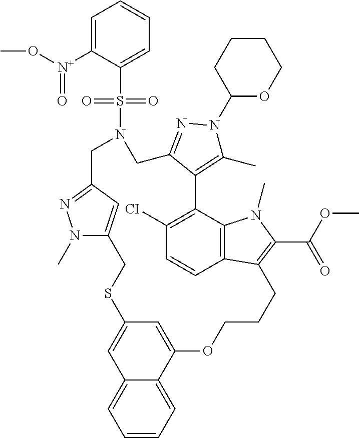

A solution of Intermediate 24 (5.3 g, 5.12 mmol) and DTBAD (4.71 g, 4 eq.) in toluene (215 mL) and THE (30 mL), previously degassed with nitrogen for 15 min, was added to a solution of PPh₃ (5.37 g, 4 eq.) in toluene (215 mL), previously degassed with nitrogen for 15 min, while stirring at 70° C. After full addition, solvents were removed under reduced pressure. The residue was purified by column flash chromatography [Biotage Isolera 1//Biotage SnapUltra Silica 100 g//heptane-EtOAc 100/0 to 0/100] to afford Intermediate 25 (6.98 g, quantitative yield).

Intermediate 26

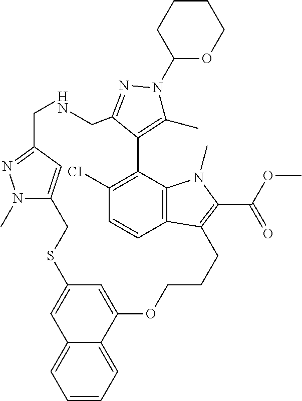

Thiophenol (5.58 mL, 10 eq.) was added dropwise to a suspension of Intermediate 25 (6.98 g, 5.44 mmol) and K₂CO₃ (7.51 g, 10 eq.) in ACN (130 mL) under nitrogen atmosphere. The reaction mixture was stirred at room temperature overnight. The reaction mixture was diluted with DCM and Celite® was added. The mixture was filtered over Celite® and the filtrate was concentrated under reduced pressure. The residue was purified by column flash chromatography [Biotage Isolera 1//Biotage SnapUltra Silica 100 g//EtOAc/Heptane:0/100 to 100/0 to EtOAc-MeOH (80/20)] to afford Intermediate 26 (2.17 g, 54% yield) as a white powder.

Intermediate 27

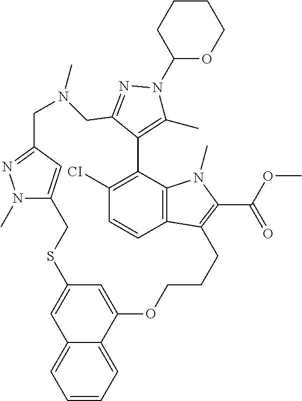

A formaldehyde solution (37% in water, 0.51 mL, 3 eq.) was added to a solution of Intermediate 26 (1.7 g, 2.3 mmol) and AcOH (2.27 mL, 17 eq.) in DCM (40 mL) at room temperature. NaBH(OAc)₃ (1.46 g, 3 eq.) was then added and the reaction mixture was stirred at room temperature for 40 min. The reaction was quenched by addition of a saturated aqueous solution of NaHCO₃ and was diluted with water and DCM. The organic layer was separated and the aqueous one was extracted with DCM. The combined organic layer was dried over MgSO₄, filtered, and evaporated to afford Intermediate 27 (1.65 g, 89% yield), used without further purification.

Intermediate 28

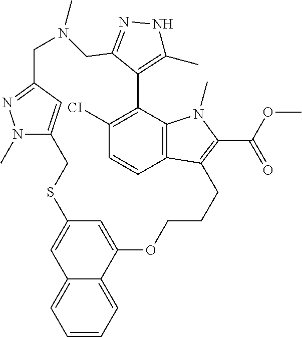

Intermediate 27 (1.65 g, 2.06 mmol) was dissolved in iPrOH (60 mL) and p-toluenesulfonic acid monohydrate (1.17 g, 3 eq.) was added. The reaction mixture was stirred at room temperature for 21 h. More p-toluenesulfonic acid monohydrate (0.59 g, 1.5 eq.) was added to the reaction mixture and it was stirred for 18 h. Again, more p-toluenesulfonic acid monohydrate (0.59 g, 1.5 eq.) was added. To push the reaction to completion, HCl (6M in iPrOH, 0.5 mL, 1.5 eq.) was added to the reaction mixture and it was stirred overnight at room temperature. Solvents were removed under reduced pressure. The residue was dissolved in EtOAc and a saturated aqueous solution of NaHCO₃ was added. The layers were separated and the aqueous layer was extracted twice with EtOAc. The combined organic layer was washed with brine, dried with MgSO₄, filtered, and evaporated to afford Intermediate 28 (1.37 g, 95% yield) used without further purification.

Intermediate 29, Intermediate 30, Intermediate 31, and Intermediate 32

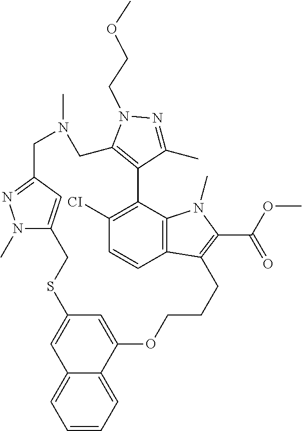

intermediate 29: Rₐ or Sₐ; one atropisomer but absolute stereochemistry undetermined

intermediate 30: Sₐ or Rₐ; one atropisomer but absolute stereochemistry undetermined

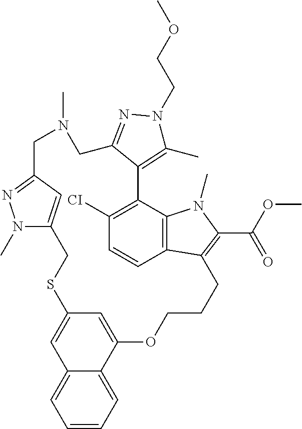

intermediate 31: Rₐ or Sₐ; one atropisomer but absolute stereochemistry undetermined

intermediate 32: Sₐ or Rₐ; one atropisomer but absolute stereochemistry undetermined

2-Bromoethyl methyl ether (29.8 μL, 2.5 eq.) was added to a mixture of Intermediate 28 (100 mg, 0.13 mmol) and Cs₂CO₃ (207 mg, 5 eq.) in DMF (5 mL) under nitrogen atmosphere. The reaction mixture was stirred at room temperature for 18 h. The reaction mixture was diluted with EtOAc and water. The organic layer was separated and washed with brine. The combined aqueous layer was back-extracted with EtOAc. The combined organic layer was dried over MgSO₄, filtered, and evaporated. The residue was purified by preparative SFC (Stationary phase: Chiralpak Diacel AD 20×250 mm, Mobile phase: CO₂, iPrOH+0.4% iPrNH₂) to give Intermediate 29 (11 mg, 12% yield), Intermediate 30 (10 mg, 11% yield), Intermediate 31 (15 mg, 16% yield), and Intermediate 32 (17 mg, 18% yield).

Intermediate 33 and Intermediate 34

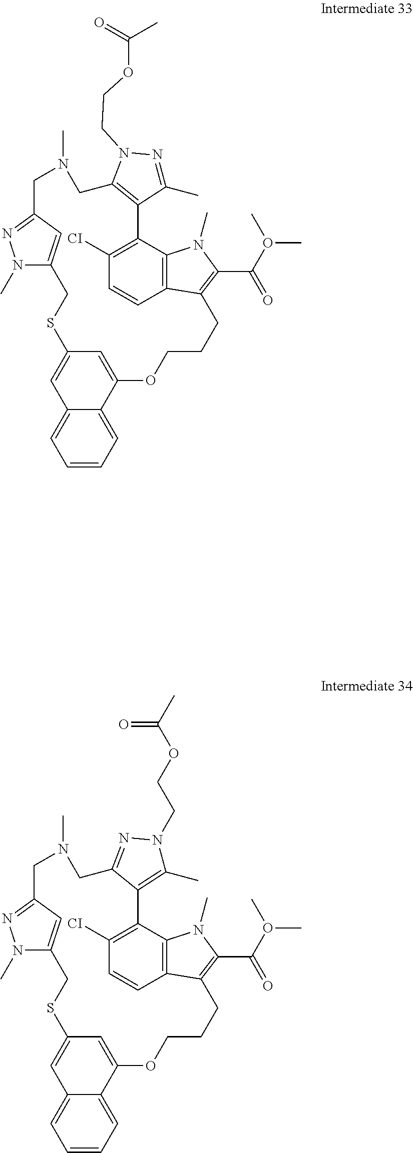

2-Bromoethyl acetate (35.1 μL, 2.5 eq.) was added to a mixture of Intermediate 28 (100 mg, 0.13 mmol) and Cs₂CO₃ (206.9 mg, 5 eq.) in DMF (5 mL) under nitrogen atmosphere. The reaction was stirred at room temperature for 18 h. The reaction mixture was diluted with EtOAc and water. The organic layer was separated and washed with brine. The combined aqueous layer was back-extracted with EtOAc. The combined organic layer was dried over MgSO₄, filtered, and evaporated to afford a 1:1 mixture of Intermediate 33 and Intermediate 34 (137 mg, quantitative). This crude mixture was used in the next step without further purification.

Intermediate 35, Intermediate 36, Intermediate 37, and Intermediate 38

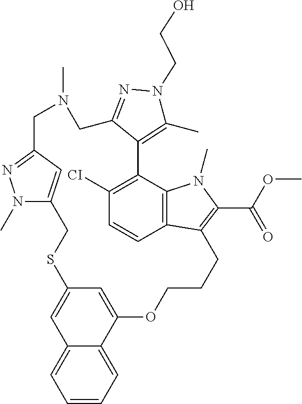

intermediate 35: Rₐ or Sₐ; one atropisomer but absolute stereochemistry undetermined

intermediate 36: Sₐ or Rₐ; one atropisomer but absolute stereochemistry undetermined

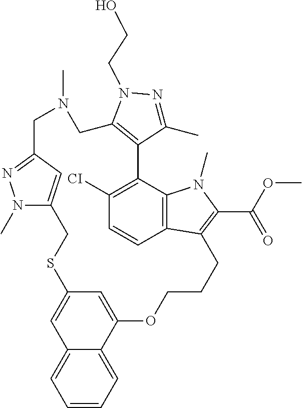

intermediate 37: Rₐ or Sₐ; one atropisomer but absolute stereochemistry undetermined

intermediate 38: Sₐ or Rₐ; one atropisomer but absolute stereochemistry undetermined

Ammonia (0.26 mL, 14.3 eq., 7 M in MeOH) was added to a solution of the 1:1 mixture of Intermediate 33 and Intermediate 34 (96 mg, 0.12 mmol). The reaction mixture was stirred overnight at room temperature. The solvent was evaporated and the residue was purified by preparative SFC (Stationary phase: Chiralpak Daicel ID 20×250 mm, Mobile phase: CO₂, iPrOH+0.4% iPrNH₂) to give Intermediate 35 (17.9 mg, 20% yield), Intermediate 36 (16.2 mg, 18% yield), Intermediate 37 (19.7 mg, 22% yield), and Intermediate 38 (17 mg, 19% yield)

Preparation of Compounds

Compound 1

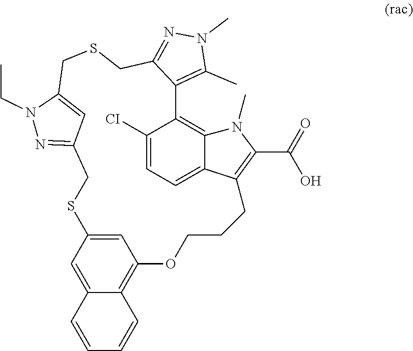

A solution of LiOH (27 mg, 15 eq.) in water (0.9 mL) was added to a solution of Intermediate 11 (61 mg, 0.08 mmol) in THE (1.8 mL) and MeOH (1.8 mL). The reaction mixture was stirred at 60° C. for 4 h. The mixture was cooled to room temperature, diluted with MeOH, and directly injected into preparative HPLC (Stationary phase: RP XBridge Prep C18 OBD-10 μm, 30×150 mm, Mobile phase: 0.25% NH₄HCO₃ solution in water, CH₃CN) to give Compound 1 (37 mg, 70% yield) as a white solid.

LC-MS: RT (min): 1.86, MW: 685.0, [MH]⁺ 686, [MH]⁺ 684 (Method: 4)

¹H NMR (400 MHz, DMSO-d6) δ ppm 1.03 (t, J=7.2 Hz, 3H), 1.95 (s, 3H), 2.12-2.24 (m, 1H), 2.30-2.42 (m, 1H), 3.07-3.19 (m, 3H), 3.38-3.48 (m, 3H), 3.50 (s, 3H), 3.60-3.71 (m, 1H), 3.72-3.83 (m, 4H), 3.85-3.97 (m, 3H), 4.04 (d, J=15.3 Hz, 1H), 5.06 (s, 1H), 6.78 (d, J=1.1 Hz, 1H), 6.83 (d, J=8.6 Hz, 1H), 7.41 (s, 1H), 7.45-7.53 (m, 2H), 7.54 (d, J=8.6 Hz, 1H), 7.74-7.79 (m, 1H), 8.17-8.22 (m, 1H)

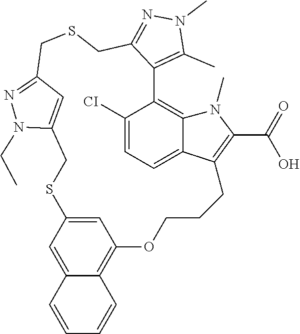

Compound 2
(rac)

A solution of LiOH (27 mg, 15 eq.) in water (0.9 mL) was added to a solution of Intermediate 12 (59 mg, 0.08 mmol) in THE (1.8 mL) and MeOH (1.8 mL). The reaction mixture was stirred at 60° C. for 4 h. The mixture was cooled to room temperature, diluted with MeOH, and directly injected into preparative HPLC (Stationary phase: RP XBridge Prep C18 OBD-10 μm, 30×150 mm, Mobile phase: 0.25% NH₄HCO₃ solution in water, CH₃CN) to give Compound 2 (42 mg, 80% yield) as a white solid.

LC-MS: RT (min): 1.86, MW: 685.0, [MH]⁺ 686, [MH]⁻ 684 (Method: 4)

¹H NMR (400 MHz, DMSO-d6) δ ppm 1.30 (t, J=7.2 Hz, 3H), 1.96 (s, 3H), 2.17-2.29 (m, 1H), 2.34-2.43 (m, 1H), 2.93 (d, J=14.1 Hz, 1H), 3.03-3.19 (m, 3H), 3.38-3.47 (m, 2H), 3.49 (s, 3H), 3.75 (s, 3H), 3.83-3.91 (m, 1H), 3.96-4.11 (m, 2H), 4.11-4.19 (m, 1H), 4.22-4.32 (m, 2H), 4.73 (s, 1H), 6.72 (d, J=0.7 Hz, 1H), 7.14 (d, J=8.6 Hz, 1H), 7.36 (s, 1H), 7.42-7.51 (m, 2H), 7.69-7.74 (m, 1H), 7.89 (d, J=8.6 Hz, 1H), 8.06-8.11 (m, 1H)

Compound 43 and Compound 44 (Atropisomers of Compound 2)

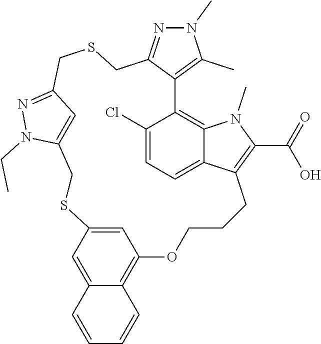 [SMILES: CCn1nc2cc1CSc1cc(c3ccccc3c1)OCCCc1c(C(=O)O)n(C)c3c(c(Cl)ccc13)-c1c(nn(C)c1C)CSC2]

Co 43: Rₐ or Sₐ atropisomer (one atropisomer; absolute stereochemistry undetermined)

Co 44: Sₐ or Rₐ atropisomer (one atropisomer; absolute stereochemistry undetermined)

Compound 2 (27 mg) was separated intro its atropisomers by preparative SFC (Stationary phase: Chiralpak Daicel IG 20×250 mm, Mobile phase: CO₂, EtOH+0.4% iPrNH₂) to afford Compound 43 (12 mg, yield: 43%) and Compound 44 (14 mg, yield: 50%).

Compound 43:

LC-MS: RT (min): 1.88, MW: 685.0, [MH]⁺ 686, [MH]⁻ 684 (Method: 5)

SFC: RT (min) 5.01, MW: 685.0, [MH]⁺ 686, [MH]⁻ 684 (Method: 4)

¹H NMR (400 MHz, CHLOROFORM-d) δ ppm 0.77-1.00 (m, 1H) 1.18-1.45 (m, 12H) 1.95 (br s, 1H) 2.01 (s, 3H) 2.29 (br s, 1H) 2.41 (br s, 1H) 2.59-2.76 (m, 1H) 3.11-3.39 (m, 4H) 3.51-3.79 (m, 8H) 3.79-4.06 (m, 11H) 4.23-4.51 (m, 1H) 4.92 (s, 1H) 6.30 (s, 1H) 6.95 (d, J=8.57 Hz, 1H) 7.40-7.56 (m, 4H) 7.70 (d, J=7.36 Hz, 1H) 8.28 (d, J=7.84 Hz, 1H)

Compound 44:

LC-MS: RT (min): 1.88, MW: 685.0, [MH]⁺ 686, [MH]⁻ 684 (Method: 5)

SFC: RT (min) 7.37, MW: 685.0, [MH]⁺ 686, [MH]⁻ 684 (Method: 4)

1H NMR (400 MHz, CHLOROFORM-d) δ ppm 1.35-1.42 (m, 4H) 2.05 (s, 3H) 2.19-2.37 (m, 1H) 2.42 (br s, 1H) 2.73 (d, J=13.69 Hz, 1H) 3.10 (br d, J=13.59 Hz, 3H) 3.16-3.32 (m, 5H) 3.54 (d, J=14.00 Hz, 1H) 3.57-3.64 (m, 1H) 3.68 (s, 5H) 3.76-3.83 (m, 1H) 3.83-3.87 (m, 5H) 3.90 (d, J=7.42 Hz, 2H) 3.93-4.04 (m, 3H) 5.00 (s, 1H) 6.23 (s, 1H) 6.98 (d, J=8.57 Hz, 1H) 7.47-7.53 (m, 3H) 7.55 (d, J=8.67 Hz, 1H) 7.71 (t, J=5.18 Hz, 1H) 8.28-8.33 (m, 1H)

Compound 3

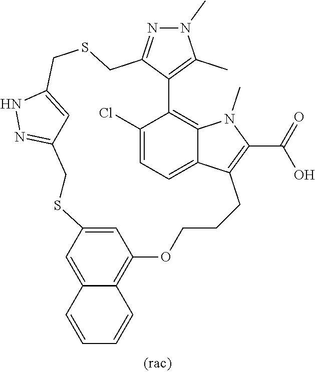 [SMILES: Cc1c2c(nn1C)CSCc1cc(n[nH]1)CSc1cc(c3ccccc3c1)OCCCc1c(C(=O)O)n(C)c3c-2c(Cl)ccc13]

A solution of LiOH (11 mg, 10 eq.) in water (0.45 mL) was added to a solution of Intermediate 10 (30 mg, 0.04 mmol) in THE (1.3 mL) and MeOH (1.3 mL). The reaction mixture was stirred at 60° C. for 2 h. After cooling to room temperature, HCl (1 M in water, 0.67 mL, 15 eq.) was added and volatiles were removed under vacuum. The residue was purified by preparative HPLC (Stationary phase: RP XBridge Prep C18 OBD-10 μm, 30×150 mm, Mobile phase: 0.25% NH₄HCO₃ solution in water, CH₃CN) to give Compound 3 (25 mg, 85% yield) as a white solid.

LC-MS: RT (min): 1.73, MW: 657.0, [MH]⁺ 658, [MH]⁻ 656 (Method: 4)

¹H NMR (400 MHz, DMSO-d6) δ ppm 1.96 (s, 3H), 2.16-2.29 (m, 1H), 2.30-2.41 (m, 1H), 2.99 (d, J=14.4 Hz, 1H), 3.04-3.14 (m, 1H), 3.20 (d, J=13.0 Hz, 1H), 3.24 (d, J=14.4 Hz, 1H), 3.38-3.47 (m, 2H), 3.49 (s, 3H), 3.76 (s, 3H), 3.84-3.92 (m, 1H), 4.02-4.18 (m, 3H), 4.82 (s, 1H), 6.70 (s, 1H), 7.05 (br d, J=8.7 Hz, 1H), 7.37 (s, 1H), 7.42-7.52 (m, 2H), 7.71-7.75 (m, 1H), 7.77 (br d, J=8.7 Hz, 1H), 8.09-9.14 (m, 1H)

The following compounds have been prepared in two steps, starting from Intermediate 10, according to analogous procedures as for Intermediate 11 or 12 (step 1), and Compound 1 or 2 (step 2):

| Co. | | Using following alkylating agent |
|---|---|---|
| No. | Structure | in step 1 |
| 4 | 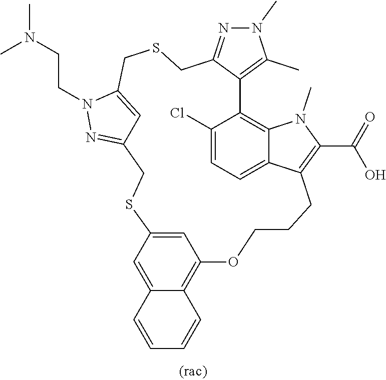 [SMILES: Cc1c2c(nn1C)CSCc1cc(nn1CCN(C)C)CSc1cc(c3ccccc3c1)OCCCc1c(C(=O)O)n(C)c3c-2c(Cl)ccc13] | 2-dimethylaminoethyl chloride hydrochloride |
| 5 | 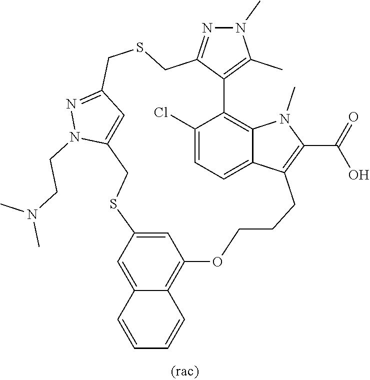 [SMILES: Cc1c2c(nn1C)CSCc1cc(n(CCN(C)C)n1)CSc1cc(c3ccccc3c1)OCCCc1c(C(=O)O)n(C)c3c-2c(Cl)ccc13] | 2-dimethylaminoethyl chloride hydrochloride |
| 6 | 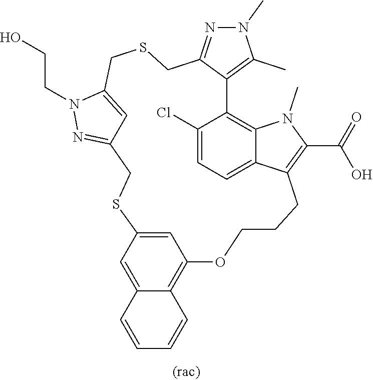 [SMILES: Cc1c2c(nn1C)CSCc1cc(nn1CCO)CSc1cc(c3ccccc3c1)OCCCc1c(C(=O)O)n(C)c3c-2c(Cl)ccc13] | 2-bromoethyl acetate |
| 7 | 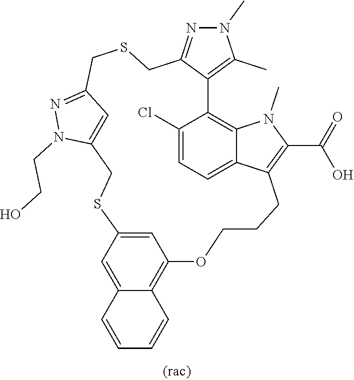 [SMILES: Cc1c2c(nn1C)CSCc1cc(n(CCO)n1)CSc1cc(c3ccccc3c1)OCCCc1c(C(=O)O)n(C)c3c-2c(Cl)ccc13] | 2-bromoethyl acetate |
| 8 | 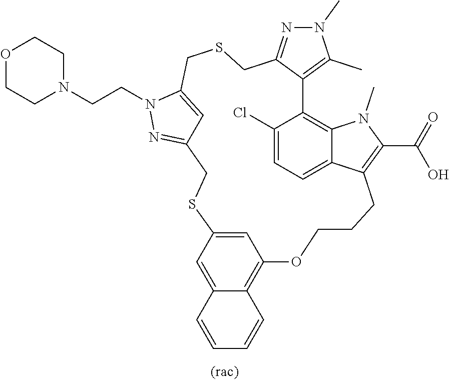 [SMILES: Cc1c2c(nn1C)CSCc1cc(nn1CCN1CCOCC1)CSc1cc(c3ccccc3c1)OCCCc1c(C(=O)O)n(C)c3c-2c(Cl)ccc13] | 4-(2-bromoethyl)morpholine |
| 9 | 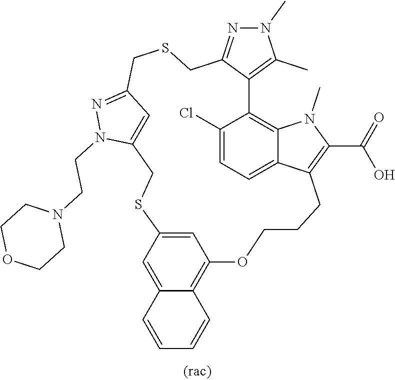 [SMILES: Cc1c2c(nn1C)CSCc1cc(n(CCN3CCOCC3)n1)CSc1cc(c3ccccc3c1)OCCCc1c(C(=O)O)n(C)c3c-2c(Cl)ccc13] | 4-(2-bromoethyl)morpholine |
| 10 | 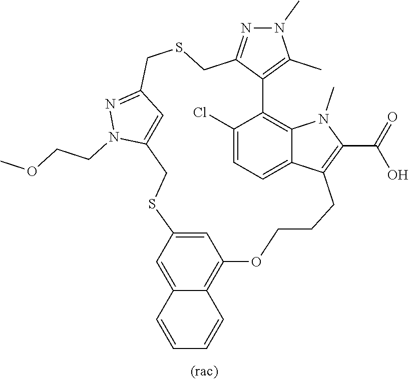 [SMILES: COCCn1nc2cc1CSc1cc(c3ccccc3c1)OCCCc1c(C(=O)O)n(C)c3c(c(Cl)ccc13)-c1c(nn(C)c1C)CSC2] | 2-bromoethyl methyl ether |
| 11 | 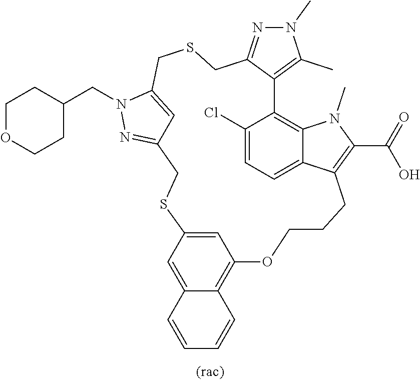 [SMILES: Cc1c2c(nn1C)CSCc1cc(nn1CC1CCOCC1)CSc1cc(c3ccccc3c1)OCCCc1c(C(=O)O)n(C)c3c-2c(Cl)ccc13] | 4-(bromomethyl)tetrahydropyran |
| 12 | 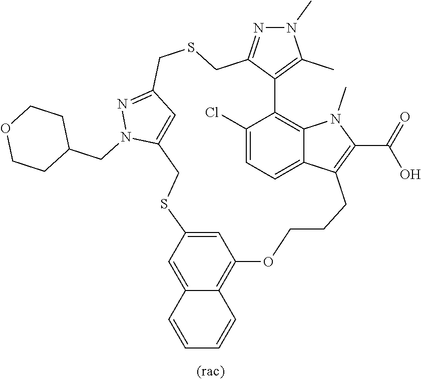 [SMILES: Cc1c2c(nn1C)CSCc1cc(n(CC3CCOCC3)n1)CSc1cc(c3ccccc3c1)OCCCc1c(C(=O)O)n(C)c3c-2c(Cl)ccc13] | 4-(bromomethyl)tetrahydropyran |

Analytical data for the Compounds shown above:

Compound 4

LC-MS: RT (min): 1.86, MW: 728.0, [MH]⁺ 729, [MH]⁻ 727 (Method: 4)

¹H NMR (400 MHz, DMSO-d6) δ ppm 1.95 (s, 3H), 2.01 (s, 6H), 2.16-2.26 (m, 1H), 2.29-2.37 (m, 3H), 3.03-3.16 (m, 3H), 3.38-3.47 (m, 3H), 3.50 (s, 3H), 3.58-3.67 (m, 1H), 3.77 (s, 3H), 3.79-3.87 (m, 1H), 3.89-4.06 (m, 4H), 5.09 (s, 1H), 6.81 (d, J=1.1 Hz, 1H), 6.92 (d, J=8.6 Hz, 1H), 7.39 (s, 1H), 7.43-7.52 (in, 2H), 7.60 (d, J=8.6 Hz, 1H), 7.72-7.77 (m, 1H), 8.13-8.18 (in, 1H) Compound 5 LC-MS: RT (min): 1.83, MW: 728.0, [MH]+729, [MH]⁻ 727 (Method: 4)

¹H NMR (400 MHz, DMSO-d6) δ ppm 1.97 (s, 3H), 2.21-2.28 (m, 7H), 2.31-2.37 (m, 1H), 2.66 (t, J=7.1 Hz, 2H), 2.93 (d, J=14.0 Hz, 1H), 3.00-3.09 (m, 1H), 3.12 (d, J=14.0 Hz, 1H), 3.15 (d, J=12.8 Hz, 1H), 3.39-3.46 (m, 2H), 3.49 (s, 3H), 3.75 (s, 3H), 3.84-3.91 (m, 1H), 4.03-4.17 (m, 3H), 4.26 (d, J=15.5 Hz, 1H), 4.34 (d, J=15.5 Hz, 1H), 4.86 (s, 1H), 6.69 (d, J=1.0 Hz, 1H), 7.12 (d, J=8.6 Hz, 1H), 7.37 (s, 1H), 7.42-7.51 (m, 2H), 7.70-7.74 (m, 1H), 7.83 (d, J=8.6 Hz, 1H), 8.06-8.10 (m, 1H)

Compound 6

LC-MS: RT (min): 1.71, MW: 701.0, [MH]⁺ 702, [MH]⁻ 701 (Method: 4)

¹H NMR (400 MHz, DMSO-d6) δ ppm 1.95 (s, 3H), 2.12-2.24 (m, 1H), 2.29-2.40 (m, 1H), 3.04-3.12 (m, 1H), 3.14 (d, J=15.0 Hz, 1H), 3.16 (d, J=13.2 Hz, 1H), 3.37-3.50 (m, 8H), 3.63-3.71 (m, 1H), 3.76 (s, 3H), 3.80-3.87 (m, 1H), 3.88-3.97 (m, 3H), 4.06 (d, J=15.2 Hz, 1H), 5.10 (s, 1H), 6.80 (d, J=8.6 Hz, 1H), 6.83 (d, J=1.3 Hz, 1H), 7.40 (s, 1H), 7.44-7.54 (m, 3H), 7.74-7.79 (m, 1H), 8.17-8.22 (m, 1H)

Compound 7

LC-MS: RT (min): 1.67, MW: 701.0, [MH]⁺ 702, [MH]⁻ 700 (Method: 4)

¹H NMR (400 MHz, DMSO-d6) δ ppm 1.97 (s, 3H), 2.16-2.28 (m, 1H), 2.29-2.38 (m, 1H), 2.92 (d, J=13.9 Hz, 1H), 3.03-3.10 (m, 1H), 3.11 (d, J=13.9 Hz, 1H), 3.19 (d, J=13.0 Hz, 1H), 3.39-3.46 (m, 2H), 3.48 (s, 3H), 3.62-3.72 (m, 2H), 3.76 (s, 3H), 3.86-3.94 (m, 1H), 3.99-4.12 (m, 3H), 4.24 (d, J=15.6 Hz, 1H), 4.34 (d, J=15.6 Hz, 1H), 4.91 (s, 1H), 6.67 (d, J=0.9 Hz, 1H), 7.08 (d, J=8.6 Hz, 1H), 7.38 (s, 1H), 7.43-7.51 (m, 2H), 7.70-7.75 (m, 1H), 7.78 (d, J=8.6 Hz, 1H), 8.08-8.12 (m, 1H)

Compound 8

LC-MS: RT (min): 1.78, MW: 770.0, [MH]⁺ 771, [MH]⁻ 769 (Method: 4)

¹H NMR (400 MHz, DMSO-d6) δ ppm 1.96 (s, 3H), 1.98-2.11 (m, 5H), 2.17-2.31 (m, 3H), 3.04 (d, J=14.6 Hz, 1H), 3.06-3.12 (m, 1H), 3.14 (d, J=13.5 Hz, 1H), 3.33 (t, J=4.7 Hz, 4H), 3.38-3.45 (m, 2H), 3.48 (d, J=13.5 Hz, 1H), 3.52 (s, 3H), 3.56-3.64 (m, 1H), 3.77 (s, 3H), 3.78-3.85 (m, 1H), 3.88-3.95 (m, 1H), 3.95-4.07 (m, 3H), 5.06 (s, 1H), 6.79 (d, J=1.3 Hz, 1H), 6.99 (d, J=8.6 Hz, 1H), 7.37 (s, 1H), 7.41-7.51 (m, 2H), 7.65 (d, J=8.7 Hz, 1H), 7.71-7.75 (m, 1H), 8.09-8.15 (m, 1H)

Compound 9

LC-MS: RT (min): 1.76, MW: 770.0, [MH]⁺ 771, [MH]⁻ 769 (Method: 4)

¹H NMR (400 MHz, DMSO-d6) δ ppm 1.97 (s, 3H), 2.18-2.29 (m, 1H), 2.35-2.47 (m, 5H), 2.64 (t, J=7.1 Hz, 2H), 2.91 (d, J=14.1 Hz, 1H), 3.03-3.11 (m, 1H), 3.12 (d, J=14.1 Hz, 1H), 3.17 (d, J=12.8 Hz, 1H), 3.41 (d, J=12.8 Hz, 1H), 3.43-3.48 (m, 1H), 3.50 (s, 3H), 3.56 (t, J=4.7 Hz, 4H), 3.76 (s, 3H), 3.84-3.92 (m, 1H), 4.04-4.16 (m, 3H), 4.27 (d, J=15.7 Hz, 1H), 4.33 (d, J=15.7 Hz, 1H), 4.83 (s, 1H), 6.68 (d, J=1.1 Hz, 1H), 7.12 (d, J=8.6 Hz, 1H), 7.37 (s, 1H), 7.43-7.51 (m, 2H), 7.70-7.75 (m, 1H), 7.84 (d, J=8.6 Hz, 1H), 8.07-8.12 (m, 1H)

Compound 10

LC-MS: RT (min): 1.82, MW: 715.0, [MH]⁺ 716, [MH]⁻ 714 (Method: 4)

¹H NMR (400 MHz, DMSO-d6) δ ppm 1.97 (s, 3H), 2.17-2.40 (m, 2H), 2.93 (d, J=14.0 Hz, 1H), 3.00-3.09 (m, 1H), 3.12 (d, J=14.0 Hz, 1H), 3.18 (dd, J=13.7, 12.8 Hz, 1H), 3.22 (s, 3H), 3.38-3.47 (m, 2H), 3.48 (s, 3H), 3.56-3.67 (m, 2H), 3.75 (s, 3H), 3.85-3.94 (m, 1H), 4.03-4.12 (m, 1H), 4.15 (t, J=5.5 Hz, 2H), 4.24 (d, J=15.6 Hz, 1H), 4.32 (d, J=15.6 Hz, 1H), 4.90 (s, 1H), 6.69 (s, 1H), 7.09 (d, J=8.6 Hz, 1H), 7.37 (s, 1H), 7.42-7.52 (m, 2H), 7.70-7.74 (m, 1H), 7.80 (d, J=8.6 Hz, 1H), 8.06-8.12 (m, 1H)

Compound 11

LC-MS: RT (min): 1.83, MW: 755.0, [MH]⁺ 756, [MH]⁻ 755 (Method: 4)

¹H NMR (400 MHz, DMSO-d6) δ ppm 0.67-0.88 (m, 4H), 1.42-1.54 (m, 1H), 1.94 (s, 3H), 2.26-2.38 (m, 2H), 2.54-2.63 (m, 1H), 2.66-2.76 (m, 1H), 2.99-3.10 (m, 4H), 3.26-3.35 (m, 2H), 3.36-3.54 (m, 7H), 3.76 (s, 3H), 3.93 (d, J=15.0 Hz, 1H), 3.99-4.07 (m, 1H), 4.08-4.19 (m, 2H), 5.18 (s, 1H), 6.88 (d, J=1.1 Hz, 1H), 7.09 (d, J=8.6 Hz, 1H), 7.30 (s, 1H), 7.39-7.48 (m, 2H), 7.67-7.71 (m, 1H), 7.80 (d, J=8.6 Hz, 1H), 8.06-8.11 (m, 1H)

Compound 12

LC-MS: RT (min): 1.82, MW: 755.0, [MH]⁺ 756, [MH]⁻ 754 (Method: 4)

¹H NMR (400 MHz, DMSO-d6) δ ppm 1.21-1.43 (m, 4H), 1.95 (s, 3H), 2.02-2.15 (m, 1H), 2.16-2.29 (m, 1H), 2.31-2.42 (m, 1H), 2.92 (d, J=14.1 Hz, 1H), 2.99-3.25 (m, 5H), 3.35-3.51 (m, 5H), 3.75 (s, 3H), 3.76-3.91 (m, 5H), 4.17-4.26 (m, 2H), 4.32 (d, J=15.6 Hz, 1H), 4.72 (s, 1H), 6.75 (s, 1H), 7.14 (d, J=8.7 Hz, 1H), 7.33 (s, 1H), 7.41-7.51 (m, 2H), 7.68-7.73 (m, 1H), 7.93 (d, J=8.7 Hz, 1H), 8.05-8.11 (m, 1H)

Compound 23 and Compound 24

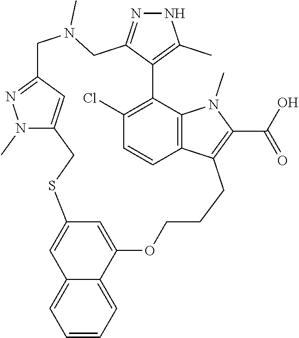 [SMILES: Cc1[nH]nc2c1-c1c(Cl)ccc3c(c(C(=O)O)n(C)c13)CCCOc1cc(cc3ccccc13)SCc1cc(nn1C)CN(C)C2]

Co 23: Rₐ or Sₐ atropisomer (one atropisomer; absolute stereochemistry undetermined)

Co 24: Sₐ or Rₐ atropisomer (one atropisomer; absolute stereochemistry undetermined)

LiOH (45 mg, 15 eq.) was added to a solution of Intermediate 28 (100 mg, 0.13 mmol) in water (1.5 mL), THE (3 mL) and MeOH (3 mL). The reaction mixture was stirred at 60° C. for 1.5 h. The solvents were evaporated and the residue was purified by preparative SFC (Stationary phase: Chiralpak Diacel AD 20×250 mm, Mobile phase: CO₂, EtOH-iPrOH (50-50)+0.4% iPrNH₂), to give Compound 23 (39 mg, 47% yield), and Compound 24 (37 mg, 45% yield).

Compound 23

LC-MS: RT (min): 1.70, MW: 654.20, [MH]⁺ 655, [MH]⁻ 653 (Method: 6)

SFC: Rt (min): 6.51, MW: 654.22, [MH]⁺ 655, [MH]⁻ 653 (Method: 1)

¹H NMR (400 MHz, DMSO-d6) δ ppm 1.81 (s, 3H), 1.88 (s, 3H), 2.24-2.36 (m, 2H), 2.66-2.88 (m, 3H), 3.04 (d, J=13.0 Hz, 1H), 3.34-3.39 (m, 4H), 3.40 (s, 3H), 3.77 (s, 3H), 3.88-3.96 (m, 1H), 4.22-4.34 (m, 2H), 4.51 (br d, J=15.4 Hz, 1H), 4.81 (s, 1H), 6.93 (s, 1H), 7.12 (d, J=8.6 Hz, 1H), 7.23 (s, 1H), 7.35-7.45 (m, 2H), 7.63 (d, J=7.5 Hz, 1H), 7.73 (br d, J=8.4 Hz, 1H), 7.97 (d, J=8.1 Hz, 1H)

Compound 24

LC-MS: RT (min): 1.70, MW: 654.20, [MH]⁺ 655, [MH]⁻ 653 (Method: 6)

SFC: Rt (min): 7.60, MW: 654.22, [MH]⁺ 655, [MH]⁻ 653 (Method: 1)

¹H NMR (400 MHz, DMSO-d6) δ ppm 1.82 (s, 3H), 1.89 (s, 3H), 2.31 (br d, J=13.0 Hz, 2H), 2.66-2.91 (m, 3H), 3.01-3.07 (m, 1H), 3.34-3.40 (m, 3H), 3.42 (s, 3H), 3.77 (s, 3H), 3.85-3.94 (m, 1H), 4.23-4.34 (m, 2H), 4.47 (br d, J=15.8 Hz, 1H), 4.75 (br s, 1H), 6.92 (s, 1H), 7.15 (d, J=8.4 Hz, 1H), 7.24 (s, 1H), 7.34-7.46 (m, 2H), 7.64 (d, J=7.7 Hz, 1H), 7.77 (br d, J=8.6 Hz, 1H), 7.98 (d, J=8.1 Hz, 1H)

Compound 25

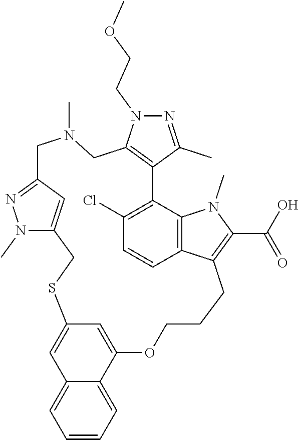 [SMILES: COCCn1nc(C)c2c1CN(C)Cc1cc(n(C)n1)CSc1cc(c3ccccc3c1)OCCCc1c(C(=O)O)n(C)c3c-2c(Cl)ccc13]

Rₐ or Sₐ atropisomer (one atropisomer but absolute stereochemistry undetermined) A solution of LiOH (5.7 mg, 15 eq.) in water was added to a solution of Intermediate 29 (11.6 mg, 0.016 mmol) in THE (0.4 mL) and MeOH (0.4 mL). The reaction mixture was stirred at room temperature for 16 h, then at 60° C. for 1 h. Aqueous HCl (1 M) and DCM were added. The aqueous layer was extracted 3 times with DCM and the combined organic layer was dried by filtration on an Extrelut NT3 column. The solvents were evaporated to afford Compound 25 (7 mg, 59% yield) as a white powder.

LC-MS: RT (min): 0.96, MW: 712.3, [MH]⁺ 713.4, [MH]⁻ 711.3 (Method: 3)

¹H NMR (400 MHz, CHLOROFORM-d) δ ppm 1.98 (s, 3H), 2.03 (s, 3H), 2.31-2.51 (m, 3H), 2.97 (d, J=14.2 Hz, 1H), 3.09-3.30 (m, 4H), 3.31 (s, 3H), 3.53 (s, 3H), 3.64-3.74 (m, 2H), 3.76 (s, 3H), 3.86-4.01 (m, 4H), 4.30 (dt, J=14.2, 5.4 Hz, 1H), 4.39-4.47 (m, 1H), 4.79 (s, 1H), 6.47 (s, 1H), 7.10 (d, J=8.6 Hz, 1H), 7.31 (s, 1H), 7.41-7.48 (m, 2H), 7.58-7.64 (m, 2H), 8.13-8.17 (m, 1H)

Compound 26

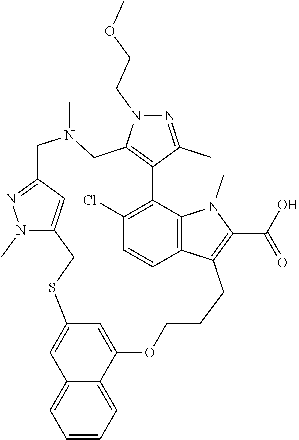 [SMILES: COCCn1nc(C)c2c1CN(C)Cc1cc(n(C)n1)CSc1cc(c3ccccc3c1)OCCCc1c(C(=O)O)n(C)c3c-2c(Cl)ccc13]

Sₐ or Rₐ atropisomer (one atropisomer; absolute stereochemistry undetermined)

Compound 26 was prepared from Intermediate 30 via an analogous reaction protocol as was used for Compound 25.

LC-MS: RT (min): 0.96, MW: 712.3, [MH]⁺ 713.3, [MH]⁻ 711.3 (Method: 3)

¹H NMR (400 MHz, CHLOROFORM-d) δ ppm 2.00 (s, 3H), 2.02 (s, 3H), 2.27-2.53 (m, 2H), 3.00 (br d, J=14.2 Hz, 1H), 3.11-3.26 (m, 3H), 3.29 (s, 3H), 3.31-3.37 (m, 1H), 3.54 (s, 3H), 3.65-3.75 (m, 3H), 3.77 (s, 3H), 3.86-4.00 (m, 4H), 4.32 (dt, J=14.3, 5.3 Hz, 1H), 4.38-4.48 (m, 1H), 4.77 (s, 1H), 6.47 (s, 1H), 7.05 (d, J=8.6 Hz, 1H), 7.29 (s, 1H), 7.40-7.48 (m, 2H), 7.55-7.62 (m, 2H), 8.12-8.18 (m, 1H)

Compound 27

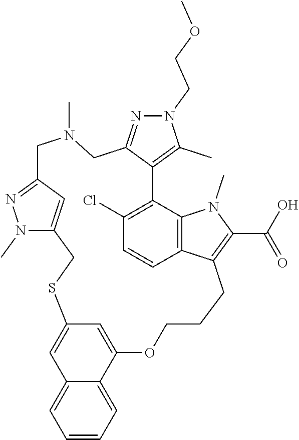 [SMILES: COCCn1nc2c(c1C)-c1c(Cl)ccc3c(c(C(=O)O)n(C)c13)CCCOc1cc(cc3ccccc13)SCc1cc(nn1C)CN(C)C2]

Rₐ or Sₐ atropisomer (one atropisomer but absolute stereochemistry undetermined)

Compound 27 was prepared from Intermediate 31 via an analogous reaction protocol as was used for Compound 25.

LC-MS: RT (min): 0.96, MW: 712.3, [MH]⁺ 713.3, [MH]⁻ 711.4 (Method: 3)

¹H NMR (400 MHz, CHLOROFORM-d) δ ppm 2.02 (br s, 3H), 2.07 (s, 3H), 2.30-2.45 (m, 2H), 2.99-3.04 (m, 1H), 3.08-3.26 (m, 3H), 3.28 (s, 3H), 3.49-3.55 (m, 1H), 3.58 (s, 3H), 3.68 (s, 3H), 3.71-3.81 (m, 4H), 3.95 (br d, J=15.0 Hz, 2H), 4.01-4.08 (m, 1H), 4.27 (br t, J=5.1 Hz, 2H), 5.08 (br s, 1H), 6.43 (s, 1H), 7.12-7.19 (m, 1H), 7.40-7.49 (m, 3H), 7.58-7.66 (m, 2H), 8.20-8.25 (m, 1H)

Compound 28

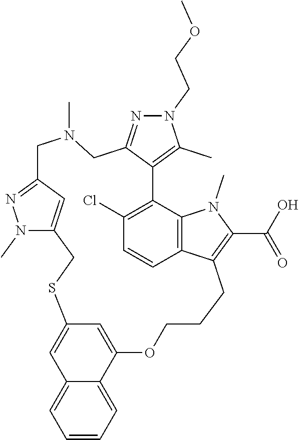 [SMILES: COCCn1nc2c(c1C)-c1c(Cl)ccc3c(c(C(=O)O)n(C)c13)CCCOc1cc(cc3ccccc13)SCc1cc(nn1C)CN(C)C2]

Sₐ or Rₐ atropisomer (one atropisomer; absolute stereochemistry undetermined)

Compound 28 was prepared from Intermediate 32 via an analogous reaction protocol as was used for Compound 25.

LC-MS: RT (min): 0.95, MW: 712.3, [MH]⁺ 713.3, [MH]⁻ 711.3 (Method: 3)

¹H NMR (400 MHz, CHLOROFORM-d) δ ppm 2.05 (br s, 3H), 2.07 (br s, 3H), 2.33 (br s, 1H), 2.42 (br s, 1H), 3.00-3.25 (m, 4H), 3.28 (s, 3H), 3.55 (br d, J=4.5 Hz, 1H), 3.59 (s, 3H), 3.64-3.71 (m, 3H), 3.71-3.81 (m, 4H), 3.95 (br d, J=15.8 Hz, 2H), 4.01-4.07 (m, 1H), 4.27 (br t, J=5.1 Hz, 2H), 5.10 (br s, 1H), 6.43 (br s, 1H), 7.09-7.17 (m, 1H), 7.38-7.49 (m, 3H), 7.58-7.68 (m, 2H), 8.23 (d, J=6.8 Hz, 1H)

Compound 37

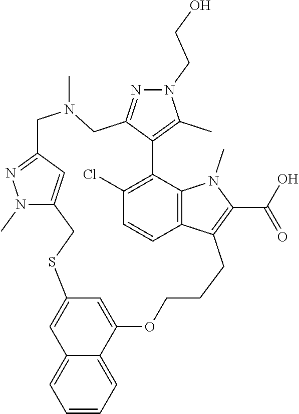 [SMILES: Cc1c2c(nn1CCO)CN(C)Cc1cc(n(C)n1)CSc1cc(c3ccccc3c1)OCCCc1c(C(=O)O)n(C)c3c-2c(Cl)ccc13]

Rₐ or Sₐ atropisomer (one atropisomer but absolute stereochemistry undetermined)

Compound 37 was prepared from Intermediate 35 via an analogous reaction protocol as was used for Compound 27.

LC-MS: RT (min) 1.71, MW: 698.2, [MH]⁺ 699.4, [MH]⁻ 697.5 (Method: 6)

SFC: RT (min) 5.99, MW: 698.2, [MH]⁺ 699, [MH]⁻ 697 (Method: 1)

¹H NMR (400 MHz, CHLOROFORM-d) δ ppm 2.06-2.17 (m, 3H), 2.29-2.44 (m, 5H), 3.10-3.49 (m, 5H), 3.55-3.66 (m, 7H), 3.70-3.86 (m, 3H), 3.88-4.00 (m, 4H), 4.20 (br s, 3H), 5.23-5.32 (m, 1H), 6.31 (s, 1H), 6.97 (d, J=8.5 Hz, 1H), 7.43-7.49 (m, 2H), 7.53 (d, J=8.5 Hz, 1H), 7.63-7.68 (m, 1H), 8.23-8.30 (m, 1H)

Compound 38

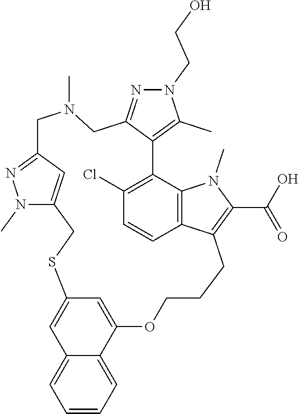 [SMILES: Cc1c2c(nn1CCO)CN(C)Cc1cc(n(C)n1)CSc1cc(c3ccccc3c1)OCCCc1c(C(=O)O)n(C)c3c-2c(Cl)ccc13]

Sₐ or Rₐ atropisomer (one atropisomer; absolute stereochemistry undetermined)

Compound 38 was prepared from Intermediate 36 via an analogous reaction protocol as was used for Compound 28.

LC-MS: RT (min) 1.71, MW: 698.2, [MH]⁺ 699.4, [MH]⁻ 697.5 (Method: 6)

SFC: RT (min) 6.63, MW: 698.24, [MH]⁺ 699, [MH]⁻ 697 (Method: 1)

¹H NMR (400 MHz, CHLOROFORM-d) δ ppm 2.10 (s, 3H), 2.29-2.47 (m, 5H), 3.10-3.48 (m, 5H), 3.61 (br d, J=11.1 Hz, 7H), 3.76 (br d, J=6.6 Hz, 2H), 3.87-3.99 (m, 4H), 4.21 (br d, J=8.9 Hz, 3H), 5.27-5.36 (m, 1H), 6.27 (s, 1H), 6.95 (br d, J=8.6 Hz, 1H), 7.43-7.56 (m, 4H), 7.64-7.70 (m, 1H), 8.25-8.32 (m, 1H)

Compound 39

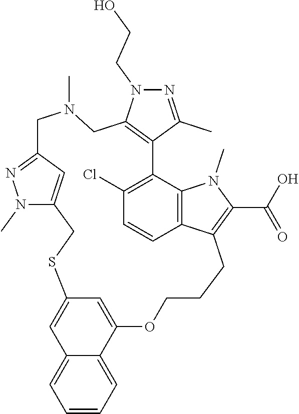 [SMILES: Cc1nn(CCO)c2c1-c1c(Cl)ccc3c(c(C(=O)O)n(C)c13)CCCOc1cc(cc3ccccc13)SCc1cc(nn1C)CN(C)C2]

Rₐ or Sₐ atropisomer (one atropisomer but absolute stereochemistry undetermined)

Compound 39 was prepared from Intermediate 37 via an analogous reaction protocol as was used for Compound 25.

LC-MS: RT (min) 1.72, MW: 698.2, [MH]⁺ 699, [MH]⁻ 697 (Method: 6)

SFC: RT (min) 6.30 min, MW: 698.24, [MH]⁺ 699, [MH]⁻ 697 (Method: 1)

¹H NMR (400 MHz, CHLOROFORM-d) δ ppm 2.03-2.08 (m, 3H), 2.13-2.20 (m, 3H), 2.33-2.41 (m, 2H), 3.05-3.26 (m, 4H), 3.42-3.48 (m, 1H), 3.49-3.54 (m, 3H), 3.58-3.67 (m, 4H), 3.73-3.77 (m, 2H), 3.78-3.83 (m, 2H), 3.85 (s, 2H), 3.96-4.06 (m, 2H), 4.25-4.43 (m, 1H), 4.92-4.98 (m, 1H), 6.26 (s, 1H), 7.00 (d, J=8.6 Hz, 1H), 7.37 (s, 1H), 7.44-7.49 (m, 2H), 7.55 (d, J=8.7 Hz, 1H), 7.60-7.67 (m, 1H), 8.16-8.23 (m, 1H)

Compound 40

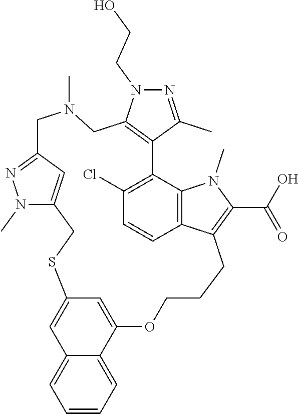 [SMILES: Cc1nn(CCO)c2c1-c1c(Cl)ccc3c(c(C(=O)O)n(C)c13)CCCOc1cc(cc3ccccc13)SCc1cc(nn1C)CN(C)C2]

Sₐ or Rₐ atropisomer (one atropisomer; absolute stereochemistry undetermined)

Compound 40 was prepared from Intermediate 38 via an analogous reaction protocol as was used for Compound 26.

LC-MS: RT (min) 1.72, MW: 698.2, [MH]⁺ 699.4, [MH]⁻ 697.5 (Method: 6)

SFC: RT (min) 6.43, MW: 698.24, [MH]⁺ 699, [MH]⁻ 697 (Method: 1)

¹H NMR (400 MHz, CHLOROFORM-d) δ ppm 2.06 (s, 3H), 2.14 (s, 3H), 2.34-2.40 (m, 2H), 3.02-3.31 (m, 4H), 3.40-3.47 (m, 1H), 3.49-3.54 (m, 3H), 3.64 (s, 4H), 3.72-3.84 (m, 3H), 3.86 (s, 2H), 4.01 (br t, J=3.5 Hz, 2H), 4.26-4.41 (m, 2H), 4.95 (s, 1H), 6.28 (s, 1H), 7.02 (d, J=8.6 Hz, 1H), 7.37 (s, 1H), 7.45-7.50 (m, 2H), 7.56 (d, J=8.6 Hz, 1H), 7.61-7.66 (m, 1H), 8.16-8.22 (m, 1H)

The following compounds were prepared from Intermediate 28 according to the details provided in the Table below.

| | | Following an | |
|---|---|---|---|
| Co. | | analogous | Reagent used in |
| No. | Structure | procedure as for | alkylating step |
| 29 | 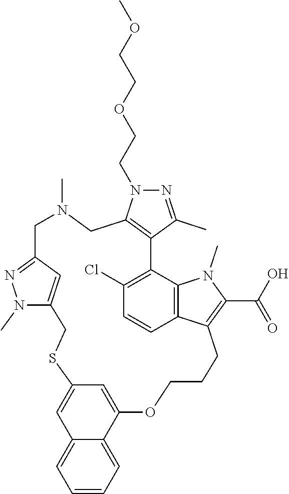 [SMILES: COCCOCCn1nc(C)c2c1CN(C)Cc1cc(n(C)n1)CSc1cc(c3ccccc3c1)OCCCc1c(C(=O)O)n(C)c3c-2c(Cl)ccc13] | Compound 25 | 1-bromo-2-(2- methoxyethoxy) ethane |
| | Rₐ or Sₐ atropisomer (one atropisomer; | | |
| | absolute stereochemistry undetermined) | | |
| 30 | 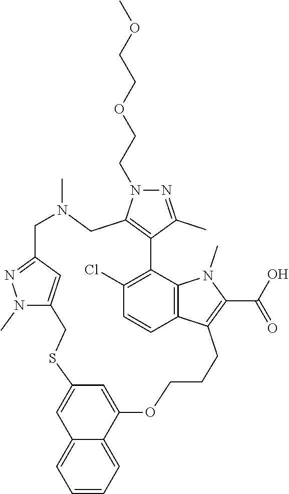 [SMILES: COCCOCCn1nc(C)c2c1CN(C)Cc1cc(n(C)n1)CSc1cc(c3ccccc3c1)OCCCc1c(C(=O)O)n(C)c3c-2c(Cl)ccc13] | Compound 26 | 1-bromo-2-(2- methoxyethoxy) ethane |
| | Sₐ or Rₐ atropisomer (one atropisomer; | | |
| | absolute stereochemistry undetermined) | | |
| 31 | 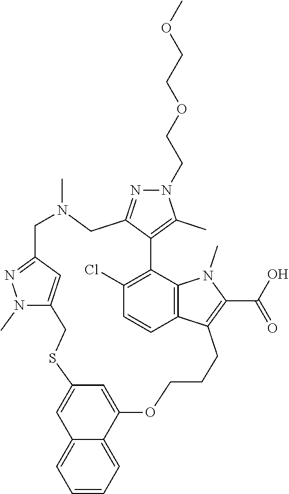 [SMILES: COCCOCCn1nc2c(c1C)-c1c(Cl)ccc3c(c(C(=O)O)n(C)c13)CCCOc1cc(cc3ccccc13)SCc1cc(nn1C)CN(C)C2] | Compound 27 | 1-bromo-2-(2- methoxyethoxy) ethane |
| | Rₐ or Sₐ atropisomer (one atropisomer; | | |
| | absolute stereochemistry undetermined) | | |
| 32 | 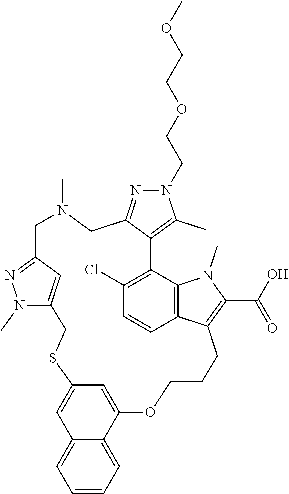 [SMILES: COCCOCCn1nc2c(c1C)-c1c(Cl)ccc3c(c(C(=O)O)n(C)c13)CCCOc1cc(cc3ccccc13)SCc1cc(nn1C)CN(C)C2] | Compound 28 | 1-bromo-2-(2- methoxyethoxy) ethane |
| | Sₐ or Rₐ atropisomer (one atropisomer; | | |
| | absolute stereochemistry undetermined) | | |
| 33 | 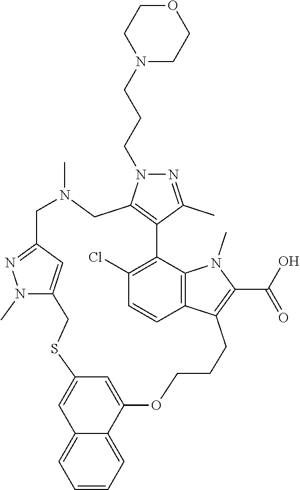 [SMILES: Cc1nn(CCCN2CCOCC2)c2c1-c1c(Cl)ccc3c(c(C(=O)O)n(C)c13)CCCOc1cc(cc3ccccc13)SCc1cc(nn1C)CN(C)C2] | Compound 25 Followed by purification via preparative HPLC (Stationary phase: RP XBridge Prep C18 OBD-10 μm, 30 × 150 mm, Mobile phase: 0.25% NH₄HCO₃ solution in water, MeOH) | 4-(3- bromopropyl)- morpholine hydrobromide |
| | Rₐ or Sₐ atropisomer (one atropisomer; | | |
| | absolute stereochemistry undetermined) | | |
| 34 | 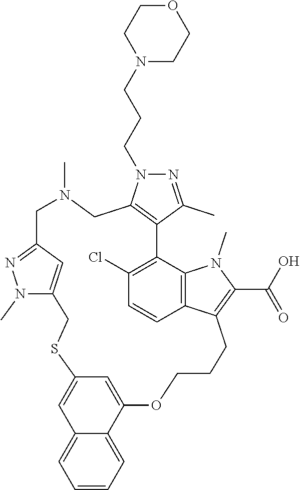 [SMILES: Cc1nn(CCCN2CCOCC2)c2c1-c1c(Cl)ccc3c(c(C(=O)O)n(C)c13)CCCOc1cc(cc3ccccc13)SCc1cc(nn1C)CN(C)C2] | Compound 26 Followed by purification via preparative HPLC (Stationary phase: RP XBridge Prep C18 OBD-10 μm, 30 × 150 mm, Mobile phase: 0.25% NH₄HCO₃ solution in water, CH₃CN) | 4-(3- bromopropyl)- morpholine hydrobromide |
| | Sₐ or Rₐ atropisomer (one atropisomer; | | |
| | absolute stereochemistry undetermined) | | |
| 35 | 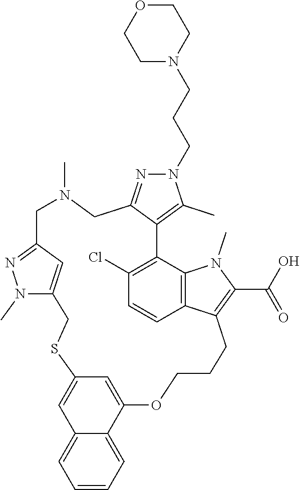 [SMILES: Cc1c2c(nn1CCCN1CCOCC1)CN(C)Cc1cc(n(C)n1)CSc1cc(c3ccccc3c1)OCCCc1c(C(=O)O)n(C)c3c-2c(Cl)ccc13] | Compound 27 Followed by purification via preparative HPLC (Stationary phase: RP XBridge Prep C18 OBD-10 μm, 30 × 150 mm, Mobile phase: 0.25% NH₄HCO₃ solution in water, MeOH) | 4-(3- bromopropyl)- morpholine hydrobromide |
| | Rₐ or Sₐ atropisomer (one atropisomer; | | |
| | absolute stereochemistry undetermined) | | |
| 36 | 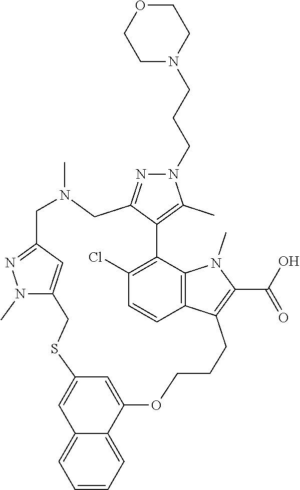 [SMILES: Cc1c2c(nn1CCCN1CCOCC1)CN(C)Cc1cc(n(C)n1)CSc1cc(c3ccccc3c1)OCCCc1c(C(=O)O)n(C)c3c-2c(Cl)ccc13] | Compound 28 Followed by purification via preparative HPLC (Stationary phase: RP XBridge Prep C18 OBD-10 μm, 30 × 150 mm, Mobile phase: 0.25% NH₄HCO₃ solution in water, MeOH) | 4-(3- bromopropyl)- morpholine hydrobromide |
| | Sₐ or Rₐ atropisomer (one atropisomer; | | |
| | absolute stereochemistry undetermined) | | |
| 41 | 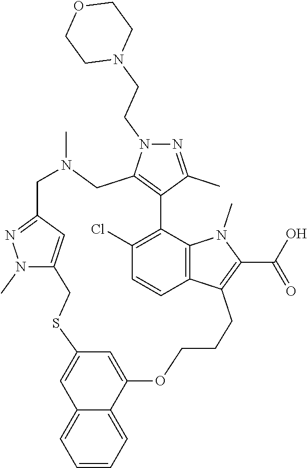 [SMILES: Cc1nn(CCN2CCOCC2)c2c1-c1c(Cl)ccc3c(c(C(=O)O)n(C)c13)CCCOc1cc(cc3ccccc13)SCc1cc(nn1C)CN(C)C2] | Compound 25 Followed by purification via preparative HPLC (Stationary phase: RP XBridge Prep C18 OBD-10 μm, 30 × 150 mm, Mobile phase: 0.25% NH₄HCO₃ solution in water, CH₃CN) | 4-(2- bromoethyl)- morpholine |
| | Rₐ or Sₐ atropisomer (one atropisomer; | | |
| | absolute stereochemistry undetermined) | | |
| 42 | 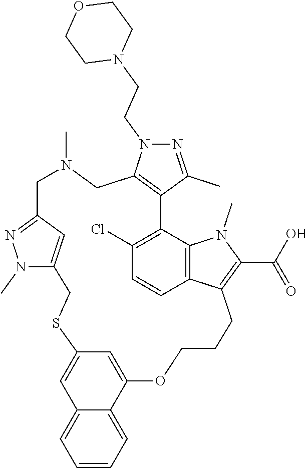 [SMILES: Cc1nn(CCN2CCOCC2)c2c1-c1c(Cl)ccc3c(c(C(=O)O)n(C)c13)CCCOc1cc(cc3ccccc13)SCc1cc(nn1C)CN(C)C2] | Compound 26 Followed by purification via Prep HPLC (Stationary phase: RP XBridge Prep C18 OBD-10 μm, 30 × 150 mm, Mobile phase: 0.25% NH₄HCO₃ solution in water, CH₃CN) | 4-(2- bromoethyl)- morpholine |
| | Sₐ or Rₐ atropisomer (one atropisomer; | | |
| | absolute stereochemistry undetermined) | | |
| 13 | 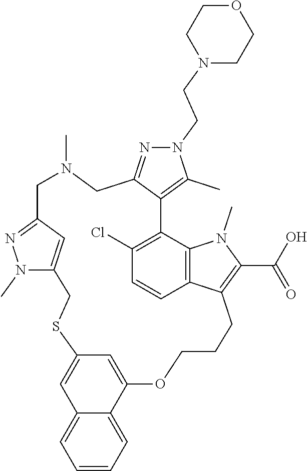 [SMILES: Cc1c2c(nn1CCN1CCOCC1)CN(C)Cc1cc(n(C)n1)CSc1cc(c3ccccc3c1)OCCCc1c(C(=O)O)n(C)c3c-2c(Cl)ccc13] | Compound 27 Followed by purification via preparative HPLC (Stationary phase: RP XBridge Prep C18 OBD-10 μm, 30 × 150 mm, Mobile phase: 0.25% NH₄HCO₃ solution in water, CH₃CN) | 4-(2- bromoethyl)- morpholine |
| | Rₐ or Sₐ atropisomer (one atropisomer; | | |
| | absolute stereochemistry undetermined) | | |
| 14 | 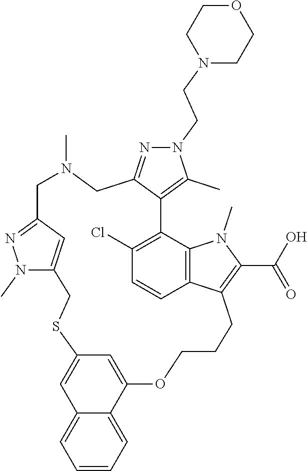 [SMILES: Cc1c2c(nn1CCN1CCOCC1)CN(C)Cc1cc(n(C)n1)CSc1cc(c3ccccc3c1)OCCCc1c(C(=O)O)n(C)c3c-2c(Cl)ccc13] | Compound 28 Followed by purification via preparative HPLC (Stationary phase: RP XBridge Prep C18 OBD-10 μm, 30 × 150 mm, Mobile phase: 0.25% NH₄HCO₃ solution in water, CH₃CN) | 4-(2- bromoethyl)- morpholine |
| | Sₐ or Rₐ atropisomer (one atropisomer; | | |
| | absolute stereochemistry undetermined) | | |
| 15 | 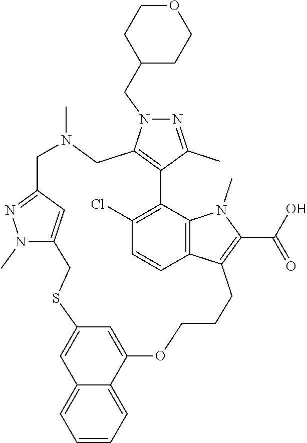 [SMILES: Cc1nn(CC2CCOCC2)c2c1-c1c(Cl)ccc3c(c(C(=O)O)n(C)c13)CCCOc1cc(cc3ccccc13)SCc1cc(nn1C)CN(C)C2] | Compound 25 Followed by purification via preparative HPLC (Stationary phase: RP XBridge Prep C18 OBD-10 μm, 30 × 150 mm, Mobile phase: 0.25% NH₄HCO₃ solution in water, MeOH) | 4- (bromomethyl)- tetrahydropyran |
| | Rₐ or Sₐ atropisomer (one atropisomer; | | |
| | absolute stereochemistry undetermined) | | |
| 16 | 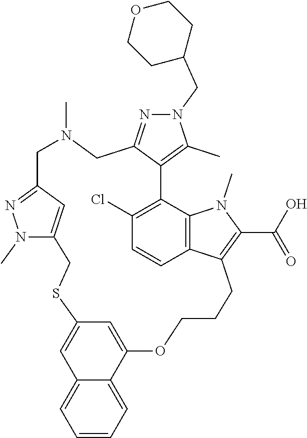 [SMILES: Cc1c2c(nn1CC1CCOCC1)CN(C)Cc1cc(n(C)n1)CSc1cc(c3ccccc3c1)OCCCc1c(C(=O)O)n(C)c3c-2c(Cl)ccc13] | Compound 27 Followed by purification via preparative HPLC (Stationary phase: RP XBridge Prep C18 OBD-10 μm, 30 × 150 mm, Mobile phase: 0.25% NH₄HCO₃ solution in water, MeOH) | 4- (bromomethyl)- tetrahydropyran |
| | Rₐ or Sₐ atropisomer (one atropisomer; | | |
| | absolute stereochemistry undetermined) | | |
| 17 | 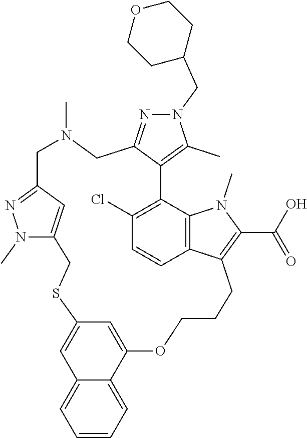 [SMILES: Cc1c2c(nn1CC1CCOCC1)CN(C)Cc1cc(n(C)n1)CSc1cc(c3ccccc3c1)OCCCc1c(C(=O)O)n(C)c3c-2c(Cl)ccc13] | Compound 28 Followed by purification via preparative HPLC (Stationary phase: RP XBridge Prep C18 OBD-10 μm, 30 × 150 mm, Mobile phase: 0.25% NH₄HCO₃ solution in water, MeOH) | 4- (bromomethyl)- tetrahydropyran |
| | Sₐ or Rₐ atropisomer (one atropisomer; | | |
| | absolute stereochemistry undetermined) | | |
| 18 | 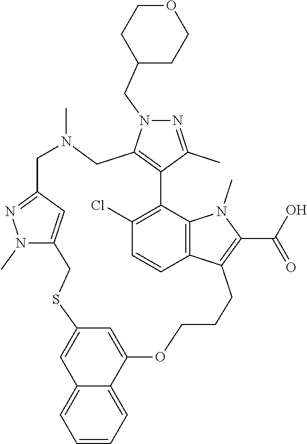 [SMILES: Cc1nn(CC2CCOCC2)c2c1-c1c(Cl)ccc3c(c(C(=O)O)n(C)c13)CCCOc1cc(cc3ccccc13)SCc1cc(nn1C)CN(C)C2] | Compound 26 Followed by purification via preparative HPLC (Stationary phase: RP XBridge Prep C18 OBD-10 μm, 30 × 150 mm, Mobile phase: 0.25% NH₄HCO₃ solution in water, MeOH) | 4- (bromomethyl)- tetrahydropyran |
| | Sₐ or Rₐ atropisomer (one atropisomer; | | |
| | absolute stereochemistry undetermined) | | |
| 19 | 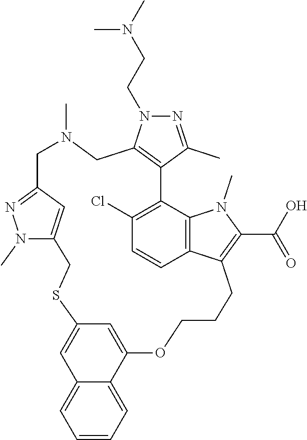 [SMILES: Cc1nn(CCN(C)C)c2c1-c1c(Cl)ccc3c(c(C(=O)O)n(C)c13)CCCOc1cc(cc3ccccc13)SCc1cc(nn1C)CN(C)C2] | Compound 25 Followed by purification via preparative HPLC (Stationary phase: RP XBridge Prep C18 OBD-10 μm, 30 × 150 mm, Mobile phase: 0.25% NH₄HCO₃ solution in water, MeOH) | 2- dimethylamino- ethyl chloride hydrochloride |
| | Rₐ or Sₐ atropisomer (one atropisomer; | | |
| | absolute stereochemistry undetermined) | | |
| 20 | 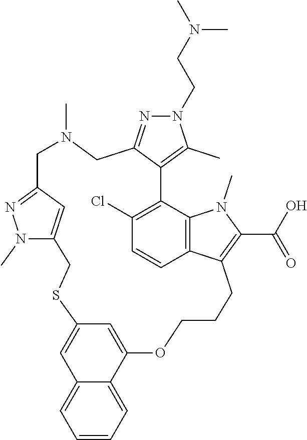 [SMILES: Cc1c2c(nn1CCN(C)C)CN(C)Cc1cc(n(C)n1)CSc1cc(c3ccccc3c1)OCCCc1c(C(=O)O)n(C)c3c-2c(Cl)ccc13] | Compound 27 Followed by purification via preparative HPLC (Stationary phase: RP XBridge Prep C18 OBD-10 μm, 30 × 150 mm, Mobile phase: 0.25% NH₄HCO₃ solution in water, MeOH) | 2- dimethylamino- ethyl chloride hydrochloride |
| | Rₐ or Sₐ atropisomer (one atropisomer; | | |
| | absolute stereochemistry undetermined) | | |
| 21 | 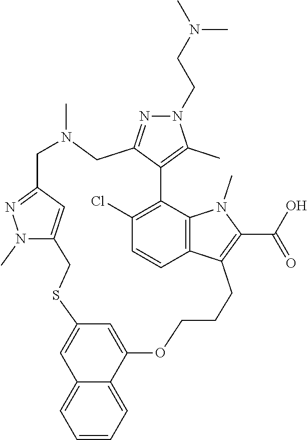 [SMILES: Cc1c2c(nn1CCN(C)C)CN(C)Cc1cc(n(C)n1)CSc1cc(c3ccccc3c1)OCCCc1c(C(=O)O)n(C)c3c-2c(Cl)ccc13] | Compound 28 Followed by purification via preparative HPLC (Stationary phase: RP XBridge Prep C18 OBD-10 μm, 30 × 150 mm, Mobile phase: 0.25% NH₄HCO₃ solution in water, MeOH) | 2- dimethylamino- ethyl chloride hydrochloride |
| | Sₐ or Rₐ atropisomer (one atropisomer; | | |
| | absolute stereochemistry undetermined) | | |
| 22 | 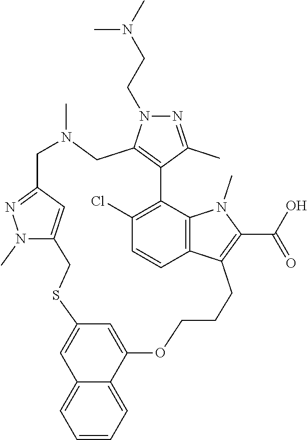 [SMILES: Cc1nn(CCN(C)C)c2c1-c1c(Cl)ccc3c(c(C(=O)O)n(C)c13)CCCOc1cc(cc3ccccc13)SCc1cc(nn1C)CN(C)C2] | Compound 26 Followed by purification via preparative HPLC (Stationary phase: RP XBridge Prep C18 OBD-10 μm, 30 × 150 mm, Mobile phase: 0.25% NHₛHCO₃ solution in water, MeOH) | 2- dimethylamino- ethyl chloride hydrochloride |
| | Sₐ or Rₐ atropisomer (one atropisomer; | | |
| | absolute stereochemistry undetermined) | | |

Analytical data for the compounds listed above:

Compound 29

LC-MS: RT (min) 1.82, MW: 756.3, [MH]⁺ 757.5, [MH]⁻ 755.6 (Method: 6)

¹H NMR (400 MHz, CHLOROFORM-d) δ ppm 1.96-2.19 (m, 6H), 2.28-2.49 (m, 2H), 3.05-3.38 (m, 7H), 3.46-3.96 (m, 18H), 4.46 (br s, 2H), 4.76-5.06 (m, 1H), 6.31-6.50 (m, 1H), 6.99-7.09 (m, 1H), 7.36 (br s, 1H), 7.41-7.50 (m, 2H), 7.57 (br d, J=8.4 Hz, 1H), 7.62 (br d, J=6.3 Hz, 1H), 8.16-8.29 (m, 1H)

Compound 30

LC-MS: RT (min) 1.82, MW: 756.3, [MH]⁺ 757.5, [MH]⁻ 755.6 (Method: 6)

¹H NMR (400 MHz, CHLOROFORM-d) δ ppm 1.98-2.19 (m, 6H), 2.24-2.54 (m, 2H), 3.06-3.37 (m, 7H), 3.45-3.98 (m, 18H), 4.46 (br s, 2H), 4.89 (br s, 1H), 6.41 (br s, 1H), 7.03 (br d, J=8.4 Hz, 1H), 7.35 (br s, 1H), 7.41-7.51 (m, 2H), 7.53-7.59 (m, 1H), 7.60-7.67 (m, 1H), 8.15-8.25 (m, 1H)

Compound 31

LC-MS: RT (min) 1.81, MW: 756.3, [MH]⁺ 757.5, [MH]⁻ 755.6 (Method: 6)

¹H NMR (400 MHz, CHLOROFORM-d) δ ppm 2.14 (br s, 3H), 2.30 (br s, 5H), 3.04-3.25 (m, 3H), 3.25-3.34 (m, 3H), 3.35-3.98 (m, 19H), 4.31 (br s, 2H), 5.51 (br s, 1H), 6.21 (br s, 1H), 6.92-7.10 (m, 1H), 7.37-7.50 (m, 3H), 7.51-7.58 (m, 1H), 7.59-7.69 (m, 1H), 8.16-8.29 (m, 1H)

Compound 32

LC-MS: RT (min) 1.81, MW: 756.3, [MH]⁺ 757.5, [MH]⁻ 755.6 (Method: 6)

¹H NMR (400 MHz, CHLOROFORM-d) δ ppm 2.16 (s, 3H), 2.33 (br s, 4H), 3.09-3.25 (m, 3H), 3.31 (s, 3H), 3.39-3.93 (m, 18H), 3.86-3.90 (m, 1H), 4.32 (br t, J=5.1 Hz, 2H), 5.67 (br s, 1H), 6.15 (br s, 1H), 7.05 (br d, J=7.9 Hz, 1H), 7.42-7.47 (m, 2H), 7.47-7.52 (m, 1H), 7.58 (br d, J=8.6 Hz, 1H), 7.62-7.68 (m, 2H), 8.20-8.28 (m, 1H)

Compound 33

LC-MS: RT (min) 1.80, MW: 781.3, [MH]⁺ 782.5, [MH]⁻ 780.6 (Method: 6)

¹H NMR (400 MHz, CHLOROFORM-d) δ ppm 1.96-2.02 (m, 6H), 2.07-2.18 (m, 2H), 2.31-2.60 (m, 8H), 3.04-3.18 (m, 4H), 3.37 (br d, J=13.2 Hz, 1H), 3.51-3.57 (m, 3H), 3.59-3.67 (m, 1H), 3.72 (s, 7H), 3.83-3.99 (m, 4H), 4.22 (br t, J=6.6 Hz, 2H), 4.74-4.79 (m, 1H), 6.44 (s, 1H), 7.01 (d, J=8.5 Hz, 1H), 7.31 (s, 1H), 7.36-7.46 (m, 2H), 7.49-7.56 (m, 1H), 7.56-7.63 (m, 1H), 8.17 (br d, J=8.3 Hz, 1H)

Compound 34

LC-MS: RT (min) 1.80, MW: 781.3, [MH]⁺ 782.5, [MH]⁻ 780.6 (Method: 6)

¹H NMR (400 MHz, CHLOROFORM-d) δ ppm 1.99 (br s, 3H), 1.99-2.02 (m, 3H), 2.05-2.20 (m, 2H), 2.29-2.60 (m, 8H), 3.03-3.20 (m, 4H), 3.30-3.43 (m, 1H), 3.49-3.56 (m, 3H), 3.56-3.66 (m, 1H), 3.71 (s, 3H), 3.72-3.80 (m, 4H), 3.82-3.98 (m, 4H), 4.22 (br t, J=6.5 Hz, 2H), 4.76 (s, 1H), 6.40-6.46 (m, 1H), 6.96-7.04 (m, 1H), 7.30 (s, 1H), 7.36-7.45 (m, 2H), 7.52 (d, J=8.6 Hz, 1H), 7.56-7.63 (m, 1H), 8.16 (br d, J=8.4 Hz, 1H)

Compound 35

LC-MS: RT (min) 1.78, MW: 781.3, [MH]⁺ 782.5, [MH]⁻ 780.7 (Method: 6)

¹H NMR (400 MHz, CHLOROFORM-d) δ ppm 1.99-2.03 (m, 3H), 2.09 (br s, 5H), 2.26-2.52 (m, 8H), 3.01-3.20 (m, 4H), 3.47-3.52 (m, 3H), 3.57 (br d, J=13.5 Hz, 2H), 3.68 (br s, 4H), 3.71 (s, 3H), 3.81-4.02 (m, 4H), 4.12 (br t, J=6.5 Hz, 2H), 5.02 (s, 1H), 6.46 (br s, 1H), 7.06 (d, J=8.5 Hz, 1H), 7.37 (s, 1H), 7.37-7.47 (m, 2H), 7.50-7.58 (m, 1H), 7.62 (br d, J=7.8 Hz, 1H), 8.17 (br d, J=8.0 Hz, 1H)

Compound 36

LC-MS: RT (min) 1.78, MW: 781.3, [MH]⁺ 782.5, [MH]⁻ 780.6 (Method: 6)

¹H NMR (400 MHz, CHLOROFORM-d) δ ppm 1.98-2.04 (m, 3H), 2.04-2.18 (m, 5H), 2.25-2.51 (m, 8H), 3.01-3.21 (m, 4H), 3.48-3.54 (m, 3H), 3.58 (br d, J=13.5 Hz, 2H), 3.68 (br t, J=4.0 Hz, 4H), 3.72 (s, 3H), 3.84-3.97 (m, 4H), 4.13 (br t, J=6.6 Hz, 2H), 5.02 (s, 1H), 6.47 (s, 1H), 7.06 (d, J=8.5 Hz, 1H), 7.37 (s, 1H), 7.38-7.47 (m, 2H), 7.53 (d, J=8.6 Hz, 1H), 7.62 (d, J=7.9 Hz, 1H), 8.17 (br d, J=8.0 Hz, 1H)

Compound 41

LC-MS: RT (min) 1.80, MW: 767.30, [MH]⁺ 768, [MH]⁻ 766 (Method: 6) H NMR (400 MHz, CHLOROFORM-d) δ ppm 1.97 (s, 4H), 1.98-2.03 (m, 2H), 2.39 (br d, J=5.1 Hz, 2H), 2.53 (br s, 3H), 2.78-2.90 (m, 2H), 2.98 (br d, J=14.1 Hz, 1H), 3.06 (br d, J=9.2 Hz, 1H), 3.13-3.22 (m, 2H), 3.31 (br d, J=14.1 Hz, 1H), 3.52 (s, 3H), 3.61 (br s, 2H), 3.65-3.70 (m, 4H), 3.76 (s, 3H), 3.88 (br s, 2H), 3.89-3.99 (m, 2H), 4.28-4.40 (m, 2H), 4.74 (s, 1H), 6.48 (s, 1H), 7.03 (d, J=8.6 Hz, 1H), 7.25-7.26 (m, 1H), 7.37-7.42 (m, 1H), 7.43 (br s, 1H), 7.51-7.57 (m, 1H), 7.55-7.60 (m, 1H), 8.08-8.18 (m, 1H)

Compound 42

LC-MS: RT (min) 1.80, MW: 767.30, [MH]⁺ 768, [MH]⁻ 766 (Method: 6)

¹H NMR (400 MHz, CHLOROFORM-d) δ ppm 1.97 (s, 3H), 2.00-2.03 (m, 3H), 2.41 (br s, 2H), 2.55 (br d, J=3.7 Hz, 4H), 2.78-2.91 (m, 2H), 2.97 (br d, J=14.3 Hz, 1H), 3.09 (br s, 1H), 3.14-3.22 (m, 2H), 3.30 (br d, J=14.1 Hz, 1H), 3.53 (s, 3H), 3.60-3.69 (m, 1H), 3.69 (br s, 4H), 3.77 (s, 3H), 3.87-3.97 (m, 2H), 3.98 (br s, 2H), 4.30-4.42 (m, 2H), 4.73 (s, 1H), 6.49 (br s, 1H), 7.04 (br d, J=8.6 Hz, 1H), 7.38 (br s, 1H), 7.39-7.45 (m, 1H), 7.51-7.57 (m, 1H), 7.52-7.58 (m, 1H), 7.58 (br s, 1H), 8.11-8.17 (m, 1H)

Compound 13

LC-MS: RT (min) 1.78, MW: 767.3, [MH]⁺ 768.5, [MH]⁻ 766.6 (Method: 6)

¹H NMR (400 MHz, CHLOROFORM-d) δ ppm 2.03-2.06 (m, 3H), 2.10 (s, 3H), 2.26-2.37 (m, 1H), 2.37-2.46 (m, 1H), 2.46-2.58 (m, 4H), 2.82-2.97 (m, 2H), 3.04-3.16 (m, 3H), 3.24 (br d, J=13.2 Hz, 1H), 3.57 (s, 3H), 3.64 (br s, 6H), 3.74 (s, 3H), 3.89 (br s, 2H), 3.94-4.05 (m, 2H), 4.26 (br t, J=5.8 Hz, 2H), 5.01 (s, 1H), 6.45-6.51 (m, 1H), 7.02-7.10 (m, 1H), 7.37-7.41 (m, 1H), 7.37-7.47 (m, 1H), 7.38-7.44 (m, 1H), 7.56 (br d, J=8.6 Hz, 1H), 7.60-7.65 (m, 1H), 8.21 (br d, J=7.5 Hz, 1H)

Compound 14

LC-MS: RT (min) 1.78, MW: 767.30, [MH]⁺ 768.5, [MH]⁻ 766.6 (Method: 6)

¹H NMR (400 MHz, CHLOROFORM-d) δ ppm 2.04 (s, 3H), 2.09 (s, 3H), 2.31 (br s, 1H), 2.43 (br s, 1H), 2.52 (br d, J=3.5 Hz, 4H), 2.89 (br d, J=4.2 Hz, 2H), 3.05-3.13 (m, 1H), 3.16 (br s, 2H), 3.23 (br d, J=13.2 Hz, 1H), 3.57 (s, 3H), 3.59-3.67 (m, 1H), 3.60-3.69 (m, 1H), 3.62-3.69 (m, 4H), 3.75 (s, 3H), 3.85-3.93 (m, 2H), 4.01 (br d, J=6.8 Hz, 2H), 4.26 (br t, J=6.2 Hz, 2H), 5.01 (s, 1H), 6.49 (s, 1H), 7.06 (d, J=8.6 Hz, 1H), 7.38 (s, 1H), 7.41 (br s, 1H), 7.41-7.47 (m, 1H), 7.56 (d, J=8.6 Hz, 1H), 7.59-7.64 (m, 1H), 8.20 (br d, J=7.5 Hz, 1H)

Compound 15

LC-MS: RT (min) 1.86, MW: 752.30, [MH]⁺ 753.5, [MH]⁻ 751.6 (Method: 6)

¹H NMR (400 MHz, CHLOROFORM-d) δ ppm 1.33-1.56 (m, 4H), 1.98 (s, 3H), 2.00 (s, 3H), 2.23 (br s, 1H), 2.41 (br d, J=5.7 Hz, 2H), 2.95 (br d, J=14.1 Hz, 1H), 3.08 (br t, J=10.2 Hz, 1H), 3.15 (s, 2H), 3.32 (br s, 2H), 3.32-3.41 (m, 1H), 3.57 (s, 3H), 3.64 (br d, J=13.9 Hz, 1H), 3.79 (s, 3H), 3.85-4.02 (m, 5H), 3.90-3.97 (m, 1 H), 4.04 (br s, 1H), 3.98-4.05 (m, 1H), 4.10 (br dd, J=13.6, 6.8 Hz, 1H), 4.75 (s, 1H), 6.51 (s, 1H), 7.09 (d, J=8.4 Hz, 1H), 7.35-7.42 (m, 1H), 7.39-7.44 (m, 1H), 7.58 (br d, J=7.7 Hz, 1H), 7.57-7.61 (m, 1H), 8.14 (br d, J=7.7 Hz, 1H)

Compound 16

LC-MS: RT (min) 1.84, MW: 752.30, [MH]⁺ 753.5, [MH]⁻ 751.5 (Method: 6)

¹H NMR (400 MHz, CHLOROFORM-d) δ ppm 1.32 (br s, 1H), 1.32-1.53 (m, 3H), 2.03 (s, 3H), 2.11 (s, 3H), 2.19-2.31 (m, 2H), 2.38 (br s, 1H), 3.03-3.11 (m, 1H), 3.03-3.17 (m, 2H), 3.24-3.31 (m, 1H), 3.27-3.37 (m, 2H), 3.57 (s, 3H), 3.64 (br s, 1H), 3.67 (br s, 1H), 3.72 (s, 3H), 3.88 (br d, J=25.7 Hz, 4H), 3.92-3.98 (m, 2H), 3.99 (br s, 2H), 5.11 (s, 1H), 6.47 (s, 1H), 7.07 (d, J=8.6 Hz, 1H), 7.37 (s, 1H), 7.37-7.42 (m, 1H), 7.39-7.45 (m, 1H), 7.55 (d, J=8.6 Hz, 1H), 7.58-7.64 (m, 1H), 8.20 (br d, J=7.9 Hz, 1H)

Compound 17

LC-MS: RT (min) 1.96, MW: 752.30, [MH]⁺ 753.5, [MH]⁻ 751.6 (Method: 6)

¹H NMR (400 MHz, CHLOROFORM-d) δ ppm 1.33-1.38 (m, 2H), 1.46 (s, 2H), 2.03 (s, 3H), 2.09 (s, 3H), 2.17-2.26 (m, 1H), 2.25-2.33 (m, 1H), 2.37 (br s, 1H), 3.01-3.13 (m, 2H), 3.06-3.13 (m, 1H), 3.20 (br s, 1H), 3.22-3.34 (m, 2H), 3.51-3.56 (m, 3H), 3.60 (br s, 1H), 3.62-3.70 (m, 1H), 3.70 (s, 3H), 3.76-3.95 (m, 4H), 3.92 (br d, J=31.0 Hz, 1H), 3.92 (br s, 2H), 3.95-3.97 (m, 1H), 5.08 (s, 1H), 6.45 (s, 1H), 7.07 (d, J=8.6 Hz, 1H), 7.36-7.42 (m, 1H), 7.36-7.41 (m, 1H), 7.41-7.47 (m, 1H), 7.49-7.57 (m, 1H), 7.63 (d, J=7.7 Hz, 1H), 8.18 (d, J=7.9 Hz, 1H)

Compound 18

LC-MS: RT (min) 1.86, MW: 752.30, [MH]⁺ 753.5, [MH]⁻ 751.6 (Method: 6)

¹H NMR (400 MHz, CHLOROFORM-d) δ ppm 1.34-1.46 (m, 3H), 1.54 (br d, J=11.7 Hz, 1H), 1.98-2.02 (m, 5H), 1.99-2.03 (m, 1H), 2.19-2.28 (m, 1H), 2.42 (br d, J=13.0 Hz, 2H), 2.95 (br d, J=13.9 Hz, 1H), 3.06-3.13 (m, 1H), 3.16 (s, 2H), 3.32-3.41 (m, 1H), 3.33-3.41 (m, 2H), 3.59 (s, 3H), 3.60-3.68 (m, 1H), 3.79 (s, 3H), 3.89-3.96 (m, 2H), 3.97 (br d, J=6.2 Hz, 4H), 3.97-4.06 (m, 1H), 3.99-4.06 (m, 1H), 4.11 (br dd, J=13.6, 6.6 Hz, 1H), 4.73 (s, 1H), 6.51 (s, 1H), 7.09 (d, J=8.6 Hz, 1H), 7.37-7.43 (m, 1H), 7.44 (br s, 1H), 7.56-7.61 (m, 1H), 7.56-7.62 (m, 1H), 8.14 (br d, J=7.5 Hz, 1H)

Compound 19

LC-MS: RT (min) 1.77, MW: 725.30, [MH]⁺ 726.5, [MH]⁻ 724.5 (Method: 6)

¹H NMR (400 MHz, CHLOROFORM-d) δ ppm 1.95 (s, 3H), 2.00 (s, 3H), 2.41 (br s, 2H), 2.52 (s, 6H), 2.88 (br d, J=14.3 Hz, 1H), 3.03-3.10 (m, 2H), 3.14-3.22 (m, 1H), 3.14-3.24 (m, 2H), 3.19-3.26 (m, 1H), 3.49 (s, 3H), 3.62-3.71 (m, 1H), 3.72 (s, 3H), 3.89 (br s, 2H), 3.93-3.99 (m, 2H), 4.43-4.52 (m, 2H), 4.68 (s, 1H), 6.53 (s, 1H), 6.97 (d, J=8.6 Hz, 1H), 7.21 (s, 1H), 7.36-7.41 (m, 1H), 7.41 (br s, 1H), 7.48-7.55 (m, 1H), 7.55 (br d, J=4.2 Hz, 1H), 8.13-8.19 (m, 1H)

Compound 20

LC-MS: RT (min) 1.76, MW: 725.3, [MH]⁺ 726.5, [MH]⁻ 724.6 (Method: 6)

¹H NMR (400 MHz, CHLOROFORM-d) δ ppm 1.94 (s, 3H), 2.02 (s, 3H), 2.27-2.40 (m, 1H), 2.34-2.47 (m, 1H), 2.42 (s, 6H), 2.95-3.05 (m, 1H), 3.02-3.10 (m, 1H), 3.07 (br d, J=13.2 Hz, 1H), 3.12-3.21 (m, 1H), 3.22 (br s, 1H), 3.53 (s, 3H), 3.58 (s, 1H), 3.60-3.69 (m, 1H), 3.78 (s, 3H), 3.87-4.02 (m, 2H), 3.95-4.02 (m, 1H), 4.05-4.13 (m, 1H), 4.32-4.43 (m, 3H), 4.88 (s, 1H), 6.56 (s, 1H), 7.06 (d, J=8.4 Hz, 1H), 7.31-7.34 (m, 1H), 7.35-7.41 (m, 1H), 7.38-7.45 (m, 1H), 7.52 (d, J=8.6 Hz, 1H), 7.59 (d, J=7.5 Hz, 1H), 8.18 (d, J=7.9 Hz, 1H)

Compound 21

LC-MS: RT (min) 1.77, MW: 725.30, [MH]⁺ 726.5, [MH]⁻ 724.5 (Method: 6)

¹H NMR (400 MHz, CHLOROFORM-d) δ ppm 1.95 (s, 3H), 2.02 (s, 3H), 2.33-2.46 (m, 3H), 2.47 (br s, 5H), 2.96-3.04 (m, 1H), 3.00-3.07 (m, 1H), 3.01-3.09 (m, 1H), 3.02-3.10 (m, 1H), 3.13-3.21 (m, 1H), 3.13-3.21 (m, 1H), 3.54 (s, 3H), 3.55-3.63 (m, 1H), 3.60-3.69 (m, 1H), 3.78 (s, 3H), 3.87-3.94 (m, 1H), 3.95-4.02 (m, 1H), 3.96-4.12 (m, 2H), 4.30-4.42 (m, 2H), 4.89 (s, 1H), 6.58 (s, 1H), 7.07 (d, J=8.4 Hz, 1H), 7.32 (s, 1H), 7.35-7.41 (m, 1H), 7.39-7.45 (m, 1H), 7.53 (d, J=8.6 Hz, 1H), 7.57-7.64 (m, 1H), 8.17 (d, J=7.9 Hz, 1H)

Compound 22

LC-MS: RT (min) 1.76, MW: 725.30, [MH]⁺ 726.5, [MH]⁻ 724.5 (Method: 6)

¹H NMR (400 MHz, CHLOROFORM-d) δ ppm 1.94 (s, 3H), 1.99 (s, 3H), 2.39 (br d, J=4.6 Hz, 2H), 2.51 (s, 6H), 2.88 (br d, J=14.1 Hz, 1H), 2.99-3.07 (m, 1H), 3.01-3.09 (m, 1H), 3.12-3.23 (m, 2H), 3.13-3.20 (m, 1H), 3.25 (s, 1H), 3.48 (s, 3H), 3.60-3.68 (m, 1H), 3.71 (s, 3H), 3.88 (br s, 2H), 3.94 (br t, J=5.7 Hz, 2H), 4.42-4.51 (m, 2H), 4.68 (s, 1H), 6.50 (s, 1H), 6.95 (d, J=8.6 Hz, 1H), 7.21 (s, 1H), 7.36-7.40 (m, 1H), 7.41 (br s, 1H), 7.49 (d, J=8.8 Hz, 1H), 7.52-7.57 (m, 1H), 8.12-8.18 (m, 1H)

Analytical Analysis

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

LCMS Method Codes (Flow expressed in mL/min; column temperature (T) in ° C.; Run time in minutes)

LC-MS Methods:

| Method | | | | | Flow | Run |
|---|---|---|---|---|---|---|
| code | Instrument | Column | Mobile phase | gradient | Column T | time |
| 1 | Agilent: | YMC: Pack | A: HCOOH | 95% A to | 2.6 | 6 |
| | 1100-DAD | ODS-AQ | 0.1% in water, | 5% A in 4.8 min, | 35 | |
| | and MSD | (3 μm, | B: CH₃CN | held for 1 min, | | |
| | | 4.6 × 50 mm) | | back to 95% | | |
| | | | | A in 0.2 min. | | |
| 2 | Agilent 1260 | YMC-pack | A: 0.1% | From 95% A to | 2.6 | 6.8 |
| | Infinity | ODS-AQ | HCOOH in | 5% A in 4.8 min, | 35 | |
| | DAD TOF-LC/MS | C18 | H₂O | held for 1.0 min, | | |
| | G6224A | (50 × 4.6 mm, | B: CH₃CN | to 95% A in 0.2 min. | | |
| | | 3 μm) | | | | |
| 3 | Waters: | Waters: | A: 10 mM | From 95% A to | 0.8 | 2 |
| | Acquity ® | BEH C18 | CH₃COONH₄ | 5% A in 1.3 min, | 55 | |
| | UPLC ® - | (1.7 μm, | in 95% H₂O + | held for 0.7 min. | | |
| | DAD and | 2.1*50 mm) | 5% CH₃CN | | | |
| | SQD | | B: CH₃CN | | | |
| 4 | Waters: | Waters: | A: 10 mM | From 100% A | 0.6 | 3.5 |
| | Acquity ® | HSS T3 | CH₃COONH₄ | to 5% A in 2.10 min, | 55 | |
| | UPLC ® - | (1.8 μm, | in 95% H₂O + | to 0% A in 0.90 min, | | |
| | DAD, SQD and | 2.1*100 mm) | 5% CH₃CN | to 5% A in 0.5 min | | |
| | ELSD | | B: CH₃CN | | | |
| 5 | Waters: | Waters: | A: 10 mM | From 100% A | 0.6 | 3.5 |
| | Acquity ® | BEH | CH₃COONH₄ | to 5% A in | 55 | |
| | UPLC ® - | (1.8 μm, | in 95% H₂O + | 2.10 min, to 0% A | | |
| | DAD and | 2.1*100 mm) | 5% CH₃CN | in 0.90 min, | | |
| | SQD | | B: CH₃CN | to 5% A | | |
| | | | | in 0.5 min | | |
| 6 | Waters: | Waters: | A: 10 mM | From 100% A | 0.7 | 3.5 |
| | Acquity ® | BEH | CH₃COONH₄ | to 5% A in | 55 | |
| | UPLC ® - | (1.8 μm, | in 95% H₂O + | 2.10 min, to 0% A | | |
| | DAD and | 2.1*100 mm) | 5% CH₃CN | in 0.90 min, | | |
| | SQD | | B: CH₃CN | to 5% A | | |
| | | | | in 0.5 min | | |

SFC-MS Methods:

The SFC measurement was performed using an Analytical Supercritical fluid chromatography (SFC) system composed by a binary pump for delivering carbon dioxide (CO2) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Analytical SFC-MS Methods (Flow expressed in mL/min; column temperature (Col T) in ° C.; Run time in minutes, Backpressure (BPR) in bars.

“iPrNH₂” means isopropylamine, “iPrOH” means 2-propanol, “EtOH” means ethanol, “min” mean minutes.

SFC Methods:

| Method | | | | Flow | Run time |
|---|---|---|---|---|---|
| code | column | mobile phase | gradient | Col T | BPR |
| 1 | Daicel | A: CO₂ | 10%-50% B | 2.5 | 9.5 |
| | Chiralpak ® AD-H | B: EtOH − | in 6 min, hold | 40 | 110 |
| | column (3.0 μm, | iPrOH + 0.2% | 3.5 min | | |
| | 150 × 4.6 mm) | iPrNH₂ | | | |
| 2 | Daicel | A: CO₂ | 10%-50% B | 2.5 | 9.5 |
| | Chiralpak ® AD-H | B: EtOH + 0.2% | in 6 min, hold | 40 | 110 |
| | column (3.0 μm, | iPrNH₂ | 3.5 min | | |
| | 150 × 4.6 mm) | | | | |
| 3 | Daicel | A: CO₂ | 10%-50% B | 2.5 | 9.5 |
| | Chiralpak ® AD-H | B: iPrOH + 0.2% | in 6 min, hold | 40 | 110 |
| | column (3.0 μm, | iPrNH₂ | 3.5 min | | |
| | 150 × 4.6 mm) | | | | |
| 4 | Daicel | A: CO₂ | 45% B | 2.5 | 9.5 |
| | Chiralpak ® IG3 | B: EtOH + | hold 6 min, to | 40 | 130 |
| | column (3.0 μm, | 0.2% iPrNH₂ | 50% in 1 min | | |
| | 150 × 4.6 mm) | | hold 2.5 min | | |

NMR

¹H NMR spectra were recorded on Bruker Avance III 400 MHz and Avance NEO 400 MHz spectrometers. CDCl₃ was used as solvent, unless otherwise mentioned. The chemical shifts are expressed in ppm relative to tetramethylsilane.

Pharmacological Analysis

### Biological Example 1

Terbium labeled Myeloid Cell Leukemia 1 (Mcl-1) homogeneous time-resolved fluorescence (HTRF) binding assay utilizing the BIM BH3 peptide (H2N-(C/Cy5Mal) WIAQELRRIGDEFN-OH) as the binding partner for Mcl-1.

Apoptosis, or programmed cell death, ensures normal tissue homeostasis, and its dysregulation can lead to several human pathologies, including cancer. Whilst the extrinsic apoptosis pathway is initiated through the activation of cell-surface receptors, the intrinsic apoptosis pathway occurs at the mitochondrial outer membrane and is governed by the binding interactions between pro- and anti-apoptotic Bcl-2 family proteins, including Mcl-1. In many cancers, the anti-apoptotic Bcl-2 protein(s), such as the Mcl-1, are upregulated, and in this way the cancer cells can evade apoptosis. Thus, inhibition of the Bcl-2 protein(s), such as Mcl-1, may lead to apoptosis in cancer cells, providing a method for the treatment of said cancers.

This assay evaluated inhibition of the BH3 domain: Mcl-1 interaction by measuring the displacement of Cy5-labeled BIM BH3 peptide (H₂N—(C/Cy5Mal) WIAQELRRIGDEFN-OH) in the HTRF assay format.

Assay Procedure

The following assay and stock buffers were prepared for use in the assay: (a) Stock buffer: 10 mM Tris-HCl, pH=7.5+150 mM NaCl, filtered, sterilized, and stored at 4° C.; and (b) 1× assay buffer, where the following ingredients were added fresh to stock buffer: 2 mM dithiothreitol (DTT), 0.0025% Tween-20, 0.1 mg/mL bovine serum albumin (BSA). The 1× Tb-Mcl-1+Cy5 Bim peptide solution was prepared by diluting the protein stock solution using the 1× assay buffer (b) to 25 pM Tb-Mcl-1 and 8 nM Cy5 Bim peptide.

Using the Acoustic ECHO, 100 nL of 100× test compound(s) were dispensed into individual wells of a white 384-well Perkin Elmer Proxiplate, for a final compound concentration of 1× and final DMSO concentration of 1%. Inhibitor control and neutral control (NC, 100 nL of 100% DMSO) were stamped into columns 23 and 24 of assay plate, respectively. Into each well of the plate was then dispensed 10 μL of the 1×Tb-Mcl-1+Cy5 Bim peptide solution. The plate was centrifuged with a cover plate at 1000 rpm for 1 minute, then incubated for 60 minutes at room temperature with plates covered.

The TR-FRET signal was read on an BMG PHERAStar FSX MicroPlate Reader at room temperature using the HTRF optic module (HTRF: excitation: 337 nm, light source: laser, emission A: 665 nm, emission B: 620 nm, integration start: 60 μs, integration time: 400 μs).

Data Analysis

The BMG PHERAStar FSX MicroPlate Reader was used to measure fluorescence intensity at two emission wavelengths—665 nm and 620 nm—and report relative fluorescence units (RFU) for both emissions, as well as a ratio of the emissions (665 nm/620 nm)*10,000. The RFU values were normalized to percent inhibition as follows:

% inhibition=(((NC−IC)−(compound−IC))/(NC−IC))*100

where IC (inhibitor control, low signal)=mean signal of 1×Tb-MCl-1+Cy5 Bim peptide+inhibitor control or 100% inhibition of Mcl-1; NC (neutral control, high signal)=mean signal 1×Tb-MCl-1+Cy5 Bim peptide with DMSO only or 0% inhibition

An 11-point dose response curve was generated to determine IC₅₀ values (using GenData) based on the following equation:

Y=Bottom+(Top−Bottom)/(1+10\hat{ }((log IC₅₀−X)*HillSlope))

where Y=% inhibition in the presence of X inhibitor concentration; Top=100% inhibition derived from the IC (mean signal of Mcl-1+inhibitor control); Bottom=0% inhibition derived from the NC (mean signal of Mcl-1+DMSO); Hillslope=Hill coefficient; and IC₅₀=concentration of compound with 50% inhibition in relation to top/neutral control (NC).

Ki=IC₅₀/(1+[L]/Kd)

In this assay [L]=8 nM and Kd=10 nM

Representative compounds of the present invention were tested according to the procedure as described above, with results as listed in the Table below. (‘NT’ means not tested). The values reported in the table below are subject to error margins associated with the assay used and the equipment.

| Compound | HTRF Ki (nM) |
|---|---|
| 3 | 0.08 |
| 11 | 0.47 |
| 12 | 0.05 |
| 10 | 0.11 |
| 9 | 0.05 |
| 8 | 0.13 |
| 7 | <0.094 |
| 6 | 0.16 |
| 4 | 0.09 |
| 5 | 0.06 |
| 1 | NT |
| 2 | 0.11 |
| 23 | 0.07 |
| 24 | 1.20 |
| 26 | 4.14 |
| 25 | 0.04 |
| 27 | 0.03 |
| 28 | 9.80 |
| 29 | 0.06 |
| 30 | 0.81 |
| 31 | 0.04 |
| 32 | 7.44 |
| 33 | 0.03 |
| 34 | 0.47 |
| 36 | 1.34 |
| 35 | 0.04 |
| 37 | 0.06 |
| 38 | 0.70 |
| 39 | 0.06 |
| 40 | 2.48 |
| 41 | 0.05 |
| 42 | 0.79 |
| 13 | 0.05 |
| 14 | 2.85 |
| 19 | 0.08 |
| 20 | 0.06 |
| 21 | 0.49 |
| 22 | 3.73 |
| 15 | 0.09 |
| 17 | 0.41 |
| 18 | 2.46 |
| 16 | 0.06 |

Representative compounds of the present invention were tested according to the procedure as described above, with results as listed in the Table below (‘NT’ means not tested). The values reported in the table below were obtained after recalibration of the equipment. The values are subject to error margins, and are averaged values over several runs of a particular compound.

| Compound | HTRF Kᵢ (nM) |
|---|---|
| 1 | 0.15 |
| 2 | 0.10 |
| 3 | 0.11 |
| 4 | 0.10 |
| 5 | 0.08 |
| 6 | 0.16 |
| 7 | NT |
| 8 | 0.08 |
| 9 | NT |
| 10 | NT |
| 11 | 0.43 |
| 12 | 0.08 |
| 13 | 0.04 |
| 14 | 2.53 |
| 15 | 0.03 |
| 16 | 0.05 |
| 17 | 0.66 |
| 18 | 3.31 |
| 19 | 0.18 |
| 20 | 0.03 |
| 21 | 0.31 |
| 22 | 3.07 |
| 23 | 0.03 |
| 24 | 1.87 |
| 25 | 0.04 |
| 26 | 5.54 |
| 27 | 0.06 |
| 28 | 9.39 |
| 29 | 0.03 |
| 30 | 0.75 |
| 31 | 0.03 |
| 32 | 8.51 |
| 33 | NT |
| 34 | 0.68 |
| 35 | NT |
| 36 | 2.06 |
| 37 | 0.04 |
| 38 | 0.48 |
| 39 | 0.05 |
| 40 | 2.61 |
| 41 | 0.03 |
| 42 | 0.79 |
| 43 | 0.04 |
| 44 | 4.06 |

### Biological Example 2

MCL-1 is a regulator of apoptosis and is highly over-expressed in tumor cells that escape cell death. The assay evaluates the cellular potency of small-molecule compounds targeting regulators of the apoptosis pathway, primarily MCL-1, Bfl-1, Bcl-2, and other proteins of the Bcl-2 family. Protein-protein inhibitors disrupting the interaction of anti-apoptotic regulators with BH3-domain proteins initiate apoptosis.

Activation of the apoptotic pathway was measured using the CellEvent™ Caspase-3/7 Green ReadyProbes™ Reagent (Thermo Fisher C10423, C10723). This assay produces a green fluorescent stain in cells that enter the apoptosis pathway. CellEvent® Caspase-3/7 Green reagent is a four amino acid peptide (DEVD) conjugated to a nucleic acid-binding dye that is non-fluorescent when not bound to DNA. The CellEvent® Caspase-3/7 Green reagent is intrinsically non-fluorescent, as the DEVD peptide inhibits binding of the dye to DNA. Upon activation of caspase-3/7 in apoptotic cells, the DEVD peptide is cleaved and the free dye can bind DNA, generating a bright green fluorescence. The activation of Caspase-3 and Caspase-7 is downstream of inhibition of MCL-1 or other apoptosis inhibiting proteins in cell lines that are dependent on them.

The live-cell readout on the IncuCyte permits tracking over time of the Caspase activation. The kinetic readout was useful as (a) it reveals differences in time of onset that can be related to differences in the mechanism of apoptosis induction, i.e. this being more direct or indirect; and (b) it allows recognition of artifacts resulting from autofluorescent or precipitating compounds. The IncuCyte readout also allows one to normalize for cell number, as the suspension cells are hard to distribute evenly.

Signals were measured every 2 h for a duration of 22 h. The ratio of the Caspase mask to the Confluence mask, per image, as raw data, was calculated and the kinetic trace for every well was exported to Genedata Screener for analysis.

In Genedata Screener values for 6 h, 12 h, and 22 h from the kinetic traces were extracted. The values were normalized against negative controls (untreated cells). A standard dose-response analysis was performed on the normalized data.

The following data was reported at each of the following three aforementioned time points: (a) The dose-response curve, (b) The qAC50 and qAC50 Mode, and (c) Max Activity.

Materials used in the assay were as listed in the Table below.

TABLE
Assay Materials
Reagent

MOLP8 cell line (mycoplasma test
negative)
ViewPlate-384 Black
CellEvent ™ Caspase-3/7 Green
Detection Reagent
Breathe-EASIERTM
DMSO (dimethyl sulfoxide)
RPMI Medium 1640 (1X) without
Phenol red and L-Glutamine
Heat Inactivated FBS (Fetal bovine
serum)
L-Glutamine solution
Gentamicin

Cells were maintained in culture medium containing 10% Heat Inactivated (HI) FBS, 2 mM L-Glutamine and 50 μg/mL Gentamycin phenol red free RPMI-1640. Cells were split at 0.4 million/mL twice a week.

On Day 1, plates containing individual wells with test compounds at 10 mM concentration, 150 nL per well. The final concentrations range from 100 μM to 10 μM compound (and no compound control) and compounds were thawed at room temperature for 1 hour. 25 μl of prewarmed medium was added into each well by multidrop (column 1, 3-22, 24), followed by addition of DMSO control (0.6% DMSO) in column 2. The plate was sealed using Breathe-Easy® sealing membrane and shaken for 30 min at room temperature to dissolve the test compound(s) in medium. The plate was then kept in the incubator for 1 hour at 37° C., 5% CO₂.

MOLP8 cells in medium at 40000/25 μl (20000/50 μl final in assay) were prepared with CellEvent™ Caspase-3/7 Green Detection Reagent at 4 μM (2 μM final in assay). Once prepared, the cells were added to the test compound plate in an amount of 20000 and the plate was immediately placed in the IncuCyte and imaging started using following settings: 10× objective, 2 s exposure time in green channel, interval of 2 h, acquisition stopped after 22 h.

For analysis in IncuCyte, a Basic Analysis protocol was defined to calculate the “confluence” and “caspase” areas from the “Phase” and “green” images, respectively, as follows: (a) Confluence: Segmentation Adjustment 1, Hole Fill 0, Adjust Size −2, No filters (b) Caspase: Top-Hat segmentation, Radius 10, Threshold 0.3 GCU, Edge Split On with sensitivity 0, Hole Fill 0, Adjust Size 1, and filter on a minimum Area of 20 μm². The analyzer is trained on a sufficient number of positive and negative control wells, as well as compound treated wells, verifying that the “confluence” layer detects both live and dead (condensed) cells. The “Caspase Area/Confluence Area” approximates the fraction of cells that are positive for the Caspase3/7 stain, calculated “Per Image”.

Assay analysis was completed in Genedata Screener, using a predefined template. More particularly, the assay-specific settings for the experiment analysis were as follows: (a) Plate layout: Negative control wells contain no compound but DMSO, and were defined to be “Neutral Control”, (b) Trace Channel: There should be one trace channel, name “Measured Channel”, of type “Measured”. This was the raw data from the IncuCyte; and (c) Layers: Three layers of the type “Aggregated: Time Series”, with the names “Mean 6 h”, “Mean 12 h” and “Mean 22 h”. They contained the mean of the measured from values from 5.5 to 6.5 hours, from 11.5 to 12.5, and from 21.5 to 22.5 hours, respectively.

Normalization and Correction: Each of the three layers was normalized to Percent-of-Control, with Neutral Control as central reference, and Stimulator Control as Scale Reference. Or, if μ_{CR} was the mean of the Central Reference, and μSC was the mean of the Scale Reference, then the normalized value was calculated as:

$$\% Activation = 100 \% ( \frac{{x}_{raw} - {μ}_{CR}}{{μ}_{SC} - {μ}_{CR}} )$$

Layer Compound Results: A standard fit model was used as below, with Sir, IC₅₀ and h as free parameters, and S₀ fixed to be 0.

$$\% Activation = {S}_{0} + \frac{{S}_{inf} - {S}_{0}}{1 + {( \frac{{IC}_{50}}{concentration} )}^{h}}$$

The Robust Z′ Factor or “RZ′ Factor” was calculated in Screener. After excluding outlier kinetic traces in control wells (see below), the RZ′ value should be RZ≥0.5 for MOLP8 cells tested at any FBS concentration, and for any of the time points (6 h, 12 h, 22 h).

The “Global SD” was calculated in Screener as the robust standard deviation of the positive or negative controls after normalization (whichever was greater). After excluding outlier kinetic traces in control wells (see below), the Global SD should be Global SD≤10 for MOLP8 cells tested at any FBS concentration, and for any of the time points (6 h, 12 h, 22 h).

Representative compounds of Formula (I) of the present invention were tested according to the procedures described in Biological Example 2, with results as listed in the Table below. ‘NT’ means not tested. The values reported in the table below are subject to error margins associated with the assay used and the equipment.

TABLE
Measured AC₅₀ for Representative Compounds of Formula (I)

| | MOLP8 Caspase 3/7 | | MOLP8 Caspase 3/7 | | MOLP8 Caspase 3/7 | |
|---|---|---|---|---|---|---|
| Compound | AC50 at 6 h (μM) | | AC50 at 12 h (μM) | | AC50 at 22 h (μM) | |
| 1 | 0.13 | (1) | 0.13 | (1) | 0.11 | (1) |
| 2 | 0.13 | (1) | 0.093 | (1) | 0.09 | (1) |
| 3 | 0.6 | (1) | 0.55 | (1) | 0.47 | (1) |
| 4 | 0.22 | (1) | 0.25 | (2) | 0.23 | (1) |
| 5 | 0.08 | (2) | 0.08 | (1) | 0.07 | (1) |
| 6 | 0.71 | (2) | 0.63 | (2) | 0.48 | (2) |
| 7 | 0.47 | (2) | 0.45 | (2) | 0.46 | (2) |
| 8 | 0.24 | (2) | 0.24 | (2) | 0.25 | (2) |
| 9 | 0.1 | (2) | 0.097 | (2) | 0.10 | (2) |
| 10 | 0.2 | (1) | 0.22 | (1) | 0.21 | (1) |
| 11 | 0.37 | (1) | 0.43 | (2) | 0.41 | (2) |
| 12 | 0.18 | (1) | 0.18 | (1) | 0.13 | (2) |
| 13 | 0.15 | (1) | 0.15 | (1) | 0.17 | (1) |
| 14 | 7.67 | (2) | 7.16 | (2) | 7.33 | (2) |
| 15 | 0.23 | (1) | 0.23 | (1) | 0.19 | (2) |
| 16 | 0.15 | (1) | 0.14 | (1) | 0.15 | (1) |
| 17 | 1.23 | (1) | 1.24 | (2) | 1.22 | (2) |
| 18 | 8.32 | (1) | 8.41 | (2) | 7.85 | (2) |
| 19 | 1.09 | (3) | 0.99 | (4) | 0.99 | (4) |
| 20 | 0.29 | (2) | 0.32 | (1) | 0.33 | (1) |
| 21 | 3.16 | (1) | 2.96 | (2) | 3.09 | (2) |
| 22 | ~17.38 | (1) | ~16.98 | (1) | ~16.98 | (1) |
| 23 | 0.45 | (2) | 0.45 | (2) | 0.42 | (2) |
| 24 | ~10.47 | (2) | 12.02 | (1) | 11.48 | (1) |
| 25 | 0.09 | (2) | 0.1 | (2) | 0.11 | (2) |
| 26 | 8.29 | (2) | 7.96 | (2) | 8.61 | (2) |
| 27 | 0.10 | (3) | 0.10 | (3) | 0.09 | (2) |
| 28 | ~20.68 | (2) | 13.41 | (2) | 11.35 | (2) |
| 29 | 0.06 | (3) | 0.062 | (3) | 0.06 | (3) |
| 30 | 2.01 | (2) | 2.14 | (2) | 2.23 | (2) |
| 31 | 0.16 | (1) | 0.16 | (1) | 0.16 | (2) |
| 32 | ~27.54 | (1) | ~21.52 | (2) | ~20.02 | (2) |
| 33 | 0.3 | (1) | 0.24 | (2) | 0.34 | (1) |
| 34 | 2.25 | (2) | 2.54 | (2) | 2.32 | (2) |
| 35 | 0.15 | (1) | 0.15 | (1) | 0.15 | (1) |
| 36 | 6.14 | (2) | 5.77 | (2) | 4.29 | (2) |
| 37 | 0.71 | (1) | 0.62 | (1) | 0.56 | (1) |
| 38 | 5.49 | (2) | 5.39 | (2) | 5.16 | (2) |
| 39 | 0.65 | (2) | 0.85 | (1) | 0.65 | (2) |
| 40 | ~26.3 | (1) | >30.2 | (2) | ~15.59 | (2) |
| 41 | 0.19 | (4) | 0.2 | (3) | 0.18 | (4) |
| 42 | 2.96 | (2) | 3.18 | (2) | 2.98 | (2) |

| | | | |
|---|---|---|---|
| 43 | NT | NT | NT |
| 44 | NT | NT | NT |

Between bracket the number of independent runs. Averaged values are reported.

### Biological Example 3

MCL-1 is a regulator of apoptosis and is highly over-expressed in tumor cells that escape cell death. The assay evaluates the cellular potency of small-molecule compounds targeting regulators of the apoptosis pathway, primarily MCL-1, Bfl-1, Bcl-2, and other proteins of the Bcl-2 family. Protein-protein inhibitors disrupting the interaction of anti-apoptotic regulators with BH3-domain proteins initiate apoptosis.

The Caspase-Glo® 3/7 Assay is a luminescent assay that measures caspase-3 and -7 activities in purified enzyme preparations or cultures of adherent or suspension cells. The assay provides a proluminescent caspase-3/7 substrate, which contains the tetrapeptide sequence DEVD. This substrate is cleaved to release aminoluciferin, a substrate of luciferase used in the production of light. Addition of the single Caspase-Glo® 3/7 Reagent in an “add-mix-measure” format results in cell lysis, followed by caspase cleavage of the substrate and generation of a “glow-type” luminescent signal.

This assay uses the MOLP-8 human multiple myeloma cell line, which is sensitive to MCL-1 inhibition.

Materials:
Perkin Elmer Envision
Multidrop 384 and small volume dispensing cassettes
Centrifuge
Countess automated cell counter
Countess counting chamber slides
Assay plate: ProxiPlate-384 Plus, White 384-shallow well Microplate
Sealing tape: Topseal A plus
T175 culture flask

| | | |
|---|---|---|
| Product | Units | Storage |

| | | | | |
|---|---|---|---|---|
| RPMI1640 (no L-Glutamine, no | 500 | mL | 4° | C. |
| phenol red) | | | | |
| Foetal Bovine Serum (FBS) (Heat | 500 | mL | 4° | C. |
| inactivated) | | | | |
| L-Glutamine (200 mM) | 100 | ml | −20° | C. |
| Gentamicin (50 mg/mL) | 100 | mL | 4° | C. |
| Caspase 3/7 Detection kit | 100 | mL | −20° | C. |
| | 10 × 10 | mL | | |

Cell Culture Media:

| MOLP8 | | |
|---|---|---|
| RPMI-1640 medium | 500 | mL |
| 20% FBS (heat inactivated) | 120 | mL |
| 2 mM L-Glutamine | 6.2 | mL |
| 50 μg/mL Gentamicin | 620 | μL |
| Assay media | | |
| RPMI-1640 medium | 500 | mL |
| 10% FBS (Heat inactivated) | 57 | mL |
| 2 mM L-Glutamine | 5.7 | mL |
| 50 μg/mL Gentamicin | 570 | μL |

Cell Culture:

Cell cultures were maintained between 0.2 and 2.0×10⁶ cells/mL. The cells were harvested by collection in 50 mL conical tubes. The cells were then pelleted at 500 g for 5 mins before removing supernatant and resuspension in fresh pre-warmed culture medium. The cells were counted and diluted as needed.

Caspase-Glo Reagent:

The assay reagent was prepared by transferring the buffer solution to the substrate vial and mixing. The solution may be stored for up to 1 week at 4° C. with negligible loss of signal.

Assay Procedure:

Compounds were delivered in assay-ready plates (Proxiplate) and stored at −20° C.

Assays always include 1 reference compound plate containing reference compounds. The plates were spotted with 40 nL of compounds (0.5% DMSO final in cells; serial dilution; 30 μM highest conc. 1/3 dilution, 10 doses, duplicates). The compounds were used at room temperature and 4 μL of pre-warmed media was added to all wells except column 2 and 23. The negative control was prepared by adding 1% DMSO in media. The positive control was prepared by adding the appropriate positive control compound in final concentration of 60 μM in media. The plate was prepared by adding 4 μL negative control to column 23, 4 μL positive control to column 2 and 4 μL cell suspension to all wells in the plate. The plate with cells was then incubated at 37° C. for 2 hours. The assay signal reagent is the Caspase-Glo solution described above, and 8 μL was added to all wells. The plates were then sealed and measured after 30 minutes.

The activity of a test compound was calculated as percent change in apoptosis induction as follows:

$$\begin{matrix}LC = median of the Low Control values \\ = Central Reference in Screener \\ = DMSO \\ = 0 \%\end{matrix} \\ \begin{matrix}HC = Median of the High Control values \\ = Scale Reference in Screener \\ = 30 µM of positive control \\ = 100 \% apoptosis induction\end{matrix} \\ \% Effect ( {AC}_{50} ) 100 - ( ( sample - LC ) / ( HC - LC ) ) * 100 \\ \% Control = ( sample / HC ) * 100 \\ \% Control min = ( sample - LC ) / ( HC - LC ) * 100$$

TABLE
Measured AC₅₀ for Representative Compounds of Formula
(I). Averaged values are reported over all runs
on all batches of a particular compound.

| | MOLP8 Caspase- |
|---|---|
| Compound | Glo AC₅₀ (nM) |
| 1 | NT |
| 2 | NT |
| 3 | 284 |
| 4 | NT |
| 5 | NT |
| 6 | 414 |
| 7 | NT |
| 8 | NT |
| 9 | NT |
| 10 | 49 |
| 11 | 385 |
| 12 | NT |
| 13 | 75 |
| 14 | 3685 |
| 15 | 67 |
| 16 | 49 |
| 17 | 564 |
| 18 | 3756 |
| 19 | 233 |
| 20 | 83 |
| 21 | 1600 |
| 22 | 14531 |
| 23 | 215 |
| 24 | 8162 |
| 25 | NT |
| 26 | 4334 |
| 27 | 36 |
| 28 | 5405 |
| 29 | 23 |
| 30 | 971 |
| 31 | 39 |
| 32 | 12056 |
| 33 | 94 |
| 34 | 1003 |
| 35 | NT |
| 36 | 2820 |
| 37 | NT |
| 38 | 3048 |
| 39 | NT |
| 40 | 10742 |
| 41 | 72 |
| 42 | 1256 |
| 43 | 63 |
| 44 | 3563 |

‘NT’ means not tested.

1. A compound of Formula (I) 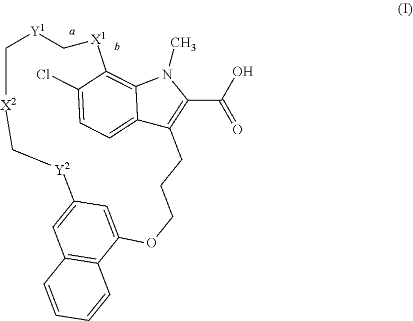
or a tautomer or a stereoisomeric form thereof, wherein
X¹ represents 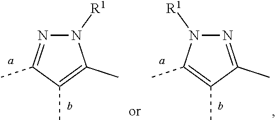
wherein ‘a’ and ‘b’ indicate how variable X¹ is attached to the remainder of the molecule;
X² represents 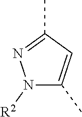
which can be attached to the remainder of the molecule in both directions;
R¹ and R² each independently represent hydrogen; methyl; or C₂-6alkyl optionally substituted with one substituent selected from the group consisting of Het¹, —OR³, and
—NR^{4a}R^{4b}; provided that at least one of R¹ and R² is other than methyl;
R³ represents hydrogen, C₁₋₄alkyl, or —C₂₋₄alkyl-O—C₁₋₄alkyl;
R^{4a} and R^{4b} are each independently selected from the group consisting of hydrogen and C₁₋₄alkyl;
Het¹ represents morpholinyl or tetrahydropyranyl;
Y¹ represents —S—, —S(═O)₂— or —N(R^{x})—;
R^{x} represents hydrogen, methyl, C₂₋₆alkyl, —C(═O)—C₁₋₆alkyl, —S(═O)₂—C₁₋₆alkyl, C₃₋₆cycloalkyl, —C(═O)—C₃₋₆cycloalkyl, or —S(═O)₂—C₃₋₆cycloalkyl; wherein C₂₋₆alkyl, —C(═O)—C₁₋₆alkyl, —S(═O)₂—C₁₋₆alkyl, C₃₋₆cycloalkyl, —C(═O)—C₃₋₆cycloalkyl, and —S(═O)₂—C₃₋₆cycloalkyl are optionally substituted with one, two or three substituents selected from the group consisting of halo, C₁₋₄alkyl and C₁₋₄alkyl substituted with one, two or three halo atoms;
Y² represents —S— or —S(═O)₂—;
or a pharmaceutically acceptable salt, or a solvate thereof.

2. The compound according to claim 1, wherein
R^{4a} and R^{4b} are C₁₋₄alkyl; and
R^{x} represents methyl.

3. The compound according to claim 1, wherein
Y¹ represent —N(R^{x})—.

4. The compound according to claim 1, wherein
X¹ represents

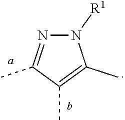

5. A pharmaceutical composition comprising a compound as claimed in claim 1 and a pharmaceutically acceptable carrier or diluent.

6. A process for preparing a pharmaceutical composition as defined in claim 5 comprising mixing a pharmaceutically acceptable carrier with a therapeutically effective amount of a compound according to claim 1.

7. (canceled)

8. (canceled)

9. (canceled)

10. A method of treating or preventing cancer, comprising administering to a subject in need thereof, a therapeutically effective amount of a compound as claimed in claim 1 or a pharmaceutical composition comprising the compound.

11. The method according to claim 10, wherein the cancer is prostate, lung, pancreatic, breast, ovarian, cervical, melanoma, B-cell chronic lymphocytic leukemia (CLL), acute myeloid leukemia (AML) or acute lymphoblastic leukemia (ALL).
